(19) United States
(12) Patent Application Publication
(10) Pub. No.: US 2023/0073368 A1
(43) Pub. Date: Mar. 9, 2023

(54) THERAPY OF RAS-DEPENDENT CANCERS

(71) Applicant: Turun Yliopisto, Turun yliopisto (FI)

(72) Inventors: Johanna LILJA, Turun yliopisto (FI); Johanna IVASKA, Turun yliopisto (FI); Olli PENTIKÄINEN, Turun yliopisto (FI); Sanna NIINIVEHMAS, Turun yliopisto (FI)

(21) Appl. No.: 17/799,253

(22) PCT Filed: Feb. 11, 2021
(86) PCT No.: PCT/FI2021/050093

§ 371 Date: Aug. 11, 2022

(30) Foreign Application Priority Data
Feb. 11, 2020 (FI) 20205139
Apr. 2, 2020 (FI) 20205343

(51) Int. Cl. C12N 15/113; G01N 33/50; G16B 15/30; A61P 35/00

(52) U.S. Cl. CPC C12N 15/113; G01N 33/5011; G16B 15/30; A61P 35/00; C12N 2310/14

(57) ABSTRACT

This invention relates to the field of cancer therapeutics. More specifically, the invention relates to inhibiting agents and methods that interfere with RAS-pathway and to their use in treating cancers.

### FIELD OF THE INVENTION

This invention relates to the field of cancer therapeutics.

### BACKGROUND OF THE INVENTION

RAS proteins are GTP-dependent switches that control and regulate signaling pathways involved in cell fate and are frequently mutated in cancer (˜30% of all human tumours)¹. Among the three RAS isoforms—HRAS, KRAS and NRAS—KRAS is the most commonly mutated gene, in 86% of RAS-driven cancers including pancreatic, lung and colorectal cancer². There is significant and compelling evidence that KRAS represents the initiating event in lung and pancreatic adenocarcinomas, two cancer types with some of the worst prognoses³. Moreover, continued function of mutant KRAS is required for tumour maintenance, and it is widely accepted that effective anti-RAS therapy will have a significant impact on cancer growth and patient survival⁴. However, despite enormous efforts in RAS research over three decades, there are no effective KRAS inhibitors in clinical use and KRAS protein remains a challenging target for cancer therapy. With KRAS mutations found in nearly all pancreatic adenocarcinoma (PDAC), this cancer type is arguably the most RAS-addicted cancer^{3,5}. Although effective targeted therapies are now available for lung and colorectal cancer, no effective targeted therapies have been found for PDAC.

RAS proteins act as binary molecular switches that cycle between active (GTP-bound) and inactive (GDP-bound) states⁶. In normal quiescent cells, RAS is predominantly GDP-bound and inactive. Extracellular stimuli triggers rapid and transient formation of RAS-GTP, leading to engagement of effector proteins that regulate diverse intracellular signaling networks³. Oncogenic KRAS mutations (typically at positions G12, G13 or Q61) prevent GAP-assisted GTP-hydrolysis and render RAS constitutively active independent of extracellular stimuli. This results in over activation of effector signaling pathways, the best known of which are the MAPK/ERK (the mitogen-activated protein (MAP) kinase/the extracellular-signal-regulated kinase) pathway and the PI3K/AKT signaling cascades, to drive inappropriate cell proliferation and survival^{4,6}.

The RAS-RAF-MEK-ERK pathway (RAS/MAPK/ERK signalling) is essential for KRAS-induced cell transformation and especially drives the growth of KRAS-mutant PDAC. While ERK activation generally stimulates growth and survival programmes, excessive ERK activation can instead cause growth arrest^{7,8}, apoptosis⁹ or senescence¹⁰. Thus, finely tuned dynamic regulation of signalling flux through this cascade is critical in dictating the cellular consequences of ERK activation and tumors with mutant oncogenes in the RAS pathway must restrain the activity of ERK1/2 to avoid toxicities and enable tumor growth¹¹.

The three RAS genes (HRAS, KRAS, and NRAS), harboring activating mutations, comprise the most frequently mutated oncogene family in cancer (27%; Catalogue of Somatic Mutations in Cancer [COSMIC] v80). KRAS is the predominant or exclusive RAS gene mutated in three of the top four neoplasms that account for cancer deaths in the US and in Europe: pancreatic ductal adenocarcinoma (PDAC), lung adenocarcinoma (LUAD) and colorectal cancer (CRC)³. The mutations render KRAS persistently GTP-bound (constitutively active) independent of extracellular stimuli, resulting in overstimulation of effector signaling pathways to drive cancer growth. Moreover, KRAS activation is one of the signaling pathways involved in resistance to EGFR tyrosine kinase inhibitors (TKIs) and monoclonal antibodies. In this context, despite EGFR inhibition, EGF-mediated downstream signaling is maintained through KRAS activation¹². Thus, it is widely accepted that an effective antiRAS therapy will have a significant impact on the inhibition of cancer growth and on improving patient survival.

SHANK family proteins (SH3 and multiple ankyrin repeat domains, SHANK, 1-3) are a family of scaffold proteins found in the postsynaptic density of excitatory synapses and are indispensable for normal brain function. Though primarily known as postsynaptic scaffold protein, the expression of SHANK3 is not limited to the central nervous system. Importantly, gene expression profiles demonstrate that SHANK3 is expressed in numerous distinct tissue and cell types. Recently, the inventors identified SHANK3 as inhibitor of major cell adhesion receptors, integrins¹³ and consequently interfering with cancer cell adhesion, spreading, migration and invasion. By solving the structure of the N-terminal reBion of SHANK3 it was found that the N-terminal SPN domain of SHANK3 protein is an unexpected Ras-association (RA) domain with high affinity for active (GTP-bound) Rap- and Ras-family GTPases.

However, despite more than three decades of intense research and industry efforts, a clinically effective anti-RAS drug was not developed. In all of these cases, the underlying rational has focused on inhibiting RAS function either directly or indirectly by, for example, preventing RAS-membrane association or by targeting downstream RAS effectors including the MEK1/2 and ERK1/2 kinases in the MAPK/ERK pathway (also known as the RAS-RAF-MEK-ERK pathway)^{2,14}. However, as exemplified by the MAPK/ERK cascade, an essential cell transformation and cancer growth promoter in PDAC, RAS signaling is not a simple linear pathway but rather a complex signaling network with multiple inputs and outputs and several feed-forward and feedback loops that complicate the therapeutic targeting of RAS effectors. In addition, the broad spectrum of RAS mutations in human cancer^{2,14} limits application of direct RAS-inhibitors to specific mutations¹⁵. Thus, it was the object of the present invention to provide new efficient cancer therapies.

### BRIEF DESCRIPTION OF THE INVENTION

The invention is based on surprising results indicating that targeting RAS-SHANK3 interaction is a highly potential therapeutic avenue for RAS-driven cancer patients. The inventors surprisingly found an alternative strategy to target RAS; based on the data, upon loss of SHANK-RAS interaction, active RAS is no longer masked or inhibited by SHANK3 thereby resulting in cytotoxic signaling and cell death specifically in RAS-driven cancers.

In particular, the invention relates to a method of preventing, treating or ameliorating a RAS-dependent cancer or a method for diminishing RAS-dependent cancer cells, the method comprising inhibiting SHANK3 function by administering a SHANK3 inhibiting agent to a mammal in need thereof.

Thus, the invention also relates to a SHANK3 inhibiting agent for use in preventing, treating or ameliorating a RAS-dependent cancer or diminishing the amount of RAS-dependent cancer cells, wherein said agent inhibits, depletes or diminishes the function of SHANK3.

As used herein, the term “SHANK3 function”, and linguistic variants thereof, means an ability of SHANK3 to inhibit active RAS. Thus, inhibition of SHANK3 function results in the activation of the RAS pathway because active RAS is no longer inhibited by SHANK3. The inhibition of SHANK3 function may be achieved through different approaches or mechanisms. For example, a SHANK3 inhibiting agent may exert its function by diminishing the ability of SHANK3 to interact with RAS. Furthermore, it is to be noted that RAS is not inhibited, i.e. RAS is “untouched”, i.e. available to interact with its downstream targets. Alternatively, a SHANK3 inhibiting agent may silence the expression of SHANK3 though for example gene editing or other methods resulting in reduced SHANK3 expression such as but not limited to RNA interference, or result in the degradation of SHANK3 protein.

In accordance with the above, SHANK3 inhibition or diminishing or depleting the function of SHANK3 leads to inhibiting, depleting, abolishing, impeding or diminishing the interaction or association of SHANK3 with a RAS isoform, a protein which activates the RAS-pathway. Preferably, the RAS isoform is HRAS, KRAS or NRAS. In some embodiments, the KRAS is KRAS encoded by a gene with one or more mutations in the KRAS gene located at a codon encoding amino acid residues at positions 5, 8, 9, 12, 13, 14, 19, 21, 22, 33, 36, 58, 59, 61, 62, 63, 68, 71, 72, 92, 117, 119, and/or 146.

In some embodiments, the SHANK3 inhibiting agent may be a binding molecule. In this context, the term “binding molecule” refers broadly to any molecule that inhibits SHANK3 function by specific binding to SHANK3. These include but are not limited to molecules that that bind to SHANK3 and inhibit its interaction with RAS. Accordingly, the binding molecule may be for example an antibody or a fragment or a variant thereof, a nanobody, an affibody, an aptamer, a peptide, such as a blocking peptide, or a small molecule. In some preferred embodiments, the inhibiting agent binds to SHANK3 through the RAS-interacting interface. The interface is preferably in the vicinity of the RAS-binding residues corresponding to the R12, K22 and R25 residues in human SHANK3 of SEQ ID NO: 1 or to the R87, K97 and R100 residues in human SHANK3 of SEQ ID NO: 36.

Thus, in a preferred embodiment, the inhibiting agent may be a binding molecule specifically binding to SHANK3. Said agent may an antibody, nanobody, affibody, an aptamer, a small-molecule inhibitor or a peptide.

In some preferred embodiments, said the SHANK3 inhibiting agent inhibits SHANK3 gene expression. Said agent may be selected from the group consisting of siRNA molecules, shRNA molecules, DsiRNA molecules, artificial miRNA precursors, and antisense oligonucleotides. In a preferred embodiment, said agent comprises a target-specific region comprising a polynucleotide having a nucleic acid sequence set forth in any one of SEQ ID NOs: 4, 5 and 17-35, or a sequence having at least 80% identity to the sequence set forth in any one of SEQ ID NOs: 4, 5 and 17-35 provided that SHANK3 inhibiting activity of the agent is retained. Furthermore, said SHANK3 inhibiting agent may be a gene editing agent. In some embodiments, the gene editing agent results in the RAS-binding domain of SHANK3 being mutated, deleted or genome edited. Consequently, functional SHANK3 is silenced or depleted resulting in activation of the RAS pathway.

Preferably, the cancer to be treated in accordance with the present invention involves an overactive, i.e. activated, RAS-MAPK.

Preferably, the cancer to be treated may be pancreatic cancer, lung cancer, colorectal cancer, ovarian cancer, melanoma, urinary bladder carcinoma, thyroid carcinoma, hematopoietic malignancy, liver carcinoma, breast cancer, neuroblastoma, cervix adenocarcinoma, head and neck carcinoma, stomach cancer, biliary tract adenocarcinoma, angiosarcoma, malignant fibrous histiocytoma, or any other cancer that is RAS-dependent, RAS-driven or has a mutation upstream of RAS pathway, more preferably a pancreatic cancer or lung cancer.

In an additional aspect, the invention relates to a method for identifying a candidate compound for treatment of RAS dependent cancer, the method comprising:

i. contacting a SHANK3 polypeptide and a RAS polypeptide with a test compound,

ii. determining whether the test compound reduces binding between SHANK3 and RAS, and

iii. identifying the test compound as a candidate compound for treatment of RAS dependent cancers, if said binding is reduced by at least 10%, preferably by at least 20%, more preferably by at least 30%.

In this and other contexts of the invention, SHANK3 may have at least 80% identity, preferably at least 85% identity, more preferably at least 90% identity, more preferably 95% identity, more preferably 98% identity, more preferably 99% identity to SEQ ID NO: 1, 2, 3 or 36, or comprises or consists of SEQ ID NO: 1, 2, 3 or 36. In particular, SHANK3 relates to a main isoform of SHANK3, which is depicted in SEQ ID NO: 1, 2, 3 or 36. However, SHANK3 also refers to any isoform, such as a splice variant, of SHANK3 comprising an SPN domain, the amino acid sequence of which is depicted in SEQ ID NOs; 11, 12 and 13.

In a preferred embodiment, the SHANK3 polypeptide comprises or consists of a RAS-binding domain. Preferably, the RAS-binding domain is SHANK3-SPN-domain or has at least 80% identity, preferably at least 85% identity, more preferably at least 90% identity, more preferably 95% identity, more preferably 98% identity, more preferably 99% identity to SEQ ID NO: 11, 12 or 13 or comprises or consists of SEQ ID NO.: 11, 12 or 13.

In a further preferred embodiment, the RAS polypeptide has at least 80% identity, preferably at least 85% identity, more preferably at least 90% identity, more preferably 95% identity, more preferably 98% identity, more preferably 99% identity to SEQ ID NO: 6, 7, 8, 9, 10, 15 or 16, or comprises or consists of SEQ ID NO: 6, 7, 8, 9, 10, 15 or 16. Notably, KRAS polypeptides set forth in SEQ ID NO: 8 and 9 refer to wild-type KRAS. Thus, in preferred embodiments of any aspect of the present invention concerning oncogenic KRAS, said polypeptides may comprise one or more amino acid substitutions at positions selected from the group consisting of positions 5, 8, 9, 12, 13, 14, 19, 21, 22, 33, 36, 58, 59, 61, 62, 63, 68, 71, 72, 92, 117, 119, and 146 of SEQ ID NO: 8 or 9. Examples of preferred mutations at these positions are set forth in SEQ ID NO: 15 and 16, respectively.

Preferably, in the method, SHANK3 or RAS is labelled with a detectable label, and/or SHANK3 or RAS is immobilised on a surface.

Preferably, the test compound is selected in silico or through other methods including but not limited to screening of compound libraries. Preferably, the test result is verified in a cellular assay.

In addition, the invention relates to a use of SHANK3 for identifying one or more agent to treat a RAS-dependent cancer, as well as to a use of an in silico model of SHANK for screening or identifying one or more candidate compounds for treatment of RAS-dependent cancer.

In addition, the invention relates to a kit comprising an isolated SHANK3, wherein the SHANK3 has at least 80% identity, preferably at least 85% identity, more preferably at least 90% identity, more preferably 95% identity, more preferably 98% identity, more preferably 99% identity to SEQ ID NO: 1, 2, 3, 11, 12, 13 or 36 or comprises or consists of SEQ ID NO.: 1, 2, 3, 11, 12, 13 or 36, and an isolated RAS-isoform polypeptide, wherein the RAS-isoform polypeptide has at least 80% identity, preferably at least 85% identity, more preferably at least 90% identity, more preferably 95% identity, more preferably 98% identity, more preferably 99% identity to any one of SEQ ID NO: 6, 7, 8, 9, 10, 15 or 16, or comprises or consists of SEQ ID NO.: 6, 7, 8, 9, 10, 15 or 16. In some preferred embodiments, said SEQ ID NO: 8 or 9 comprises one or more amino acid substitutions at positions selected from the group consisting of positions 5, 8, 9, 12, 13, 14, 19, 21, 22, 33, 36, 58, 59, 61, 62, 63, 68, 71, 72, 92, 117, 119, and 146. Examples of preferred mutations at these positions are set forth in SEQ ID NO: 15 and 16, respectively. The kit may be used for screening or identifying one or more agents for treating a RAS-dependent cancer.

Also provided is a computer-based method for identifying or designing a candidate compound for treatment of RAS dependent cancer, the method comprising

i. providing a spatial structure of the RAS binding domain of SHANK3, wherein said domain comprises at least amino acids, corresponding to R12 and K22 in the in the human RAS binding domain of SHANK3 (SEQ ID NO: 11), in a computer,

ii. generating a spatial structure of potential inhibitors in a computer, and

iii. selecting potential inhibitors having a structure which can bind at least one amino acid residue of said domain.

The invention relates as well to methods of preventing, treating or ameliorating RAS-dependent cancers or to methods of diminishing RAS-dependent cancer cells with all preferred embodiments as described herein.

Further aspects, embodiments, details and advantages of the present invention will become apparent from the following figures, detailed description, examples, and dependent claims.

### BRIEF DESCRIPTION OF THE DRAWINGS

In the following the invention will be described in greater detail by means of preferred embodiments with reference to the attached drawings, in which

FIG. 1. SHANK3 directly interacts and colocalizes with oncogenic KRAS

a. Illustration of SHANK3 protein (SEQ ID NOs:1, 2 and 3) domains. SPN (SEQ ID NOs:11, 12 and 13), Shank/ProSAP N-terminal domain; ARR, ankyrin repeat domain; SH3, Src homology 3 domain; PDZ, PSD-95/Discs large/ZO-1 domain; PP, proline-rich region; SAM, sterile alpha motif domain.

b. Model of the SHANK3 SPN domain in complex with active KRAS. RAS binding deficient double mutation R12E/K22D in the SPN domain is indicated.

c. SHANK3 WT-mRFP, co-expressed with GFP-tagged constitutively active (G12V) KRAS4B in HEK293 cells, was immunoprecipitated (IP) from cell lysates, and input lysates and IP samples were analysed using anti-GFP and antiSHANK3 antibodies, as indicated.

d. GFP-tagged SHANK3 SPN WT or R12E/K22D mutant, coexpressed with constitutively active (G12V) KRAS4B, was immunoprecipitated (IP) from cell lysates, and input lysates and IP samples were analysed using anti-GFP and anti-KRAS antibodies, as indicated.

e. Interaction between mRFP-tagged constitutively active (G12V) KRAS4B and GFP-tagged SHANK3 SPN WT or control vector in HEK293 cells measured by FRET. Data represent mean±s.d. Statistical analysis: unpaired two-tailed Student's t-test.

FIG. 2. SHANK3 localizes to membrane with oncogenic KRAS

GFP-SHANK3-expressing MIA Paca-2 cells plated on fibronectin-collagen demonstrate SHANK3 WT localization at the plasma membrane. The membrane localization is disrupted by R12E/K22D mutation in SHANK3. Shown are representative SIM images (bottom plane).

FIG. 3. SHANK3 inhibits oncogenic RAS-ERK signaling in cells that dependent on MAPK pathway

a, b. Representative western blot (a) and quantification (b) showing levels of ERK1/2 phosphorylation (phospho-ERK1/2 (Thr202/Y204) relative to total ERK) in HCT116 cells transiently expressing GFP-tagged control, SHANK3 SPN WT or R12E/K22D mutant. Data represent mean±s.d. Statistical analysis: unpaired two-tailed Student's t-test.

c, d. Representative confocal images (c) and quantification (d) showing levels of nuclear ERK (indicated ERK activity) in KRASG12C-mutant MIA PaCa-2 cells transiently expressing GFP-tagged control, SHANK3 SPN WT or R12E/K22D mutant. Cells were stained for total ERK (grey). Shown are confocal slices from the middle surface. Data represent mean±s.d. Statistical analysis: unpaired two-tailed Student's t-test.

FIG. 4. SHANK3 overexpression inhibits KRAS-induced transformation and tumorigenesis

a, b. Representative images (e) and quantification (f) of colony survival assay of control or stably KRASG12V expressing NIH/3T3 cells which were transiently transfected GFP control or GFP-SHANK3 SPN WT. Colony survival was graphed based on colony area (%). Data represent mean±s.d. Statistical analysis: unpaired two-tailed Student's t-test.

c, d. HCT116 cells (1×10⁶) transiently expressing GFP-tagged control, SHANK3 SPN WT or R12E/K22D mutant were implanted on in ovo CAM membranes inside a plastic ring to analyse tumor growth in vivo for 3 days. Shown are representative images (c) and quantified tumor weight (d) from two individual experiments. Data represent mean±s.d. Statistical analysis: unpaired two-tailed Student's t-test. In c, the tumor area is outlined by a dashed line.

FIG. 5. SHANK3 suppresses KRAS-induced macropinocytosis

a, b. Representative images (a) and quantification (b) of macropinocytosis (TMR-dextran updake) MIA PaCa-2 cells transiently expressing GFP-tagged control, SHANK3 SPN WT or R12E/K22D mutant. Data represent mean±s.d. Statistical analysis: unpaired two-tailed Student's t-test.

FIG. 6. SHANK3 expressed at low levels in KRAS-mutant cancer

a. Schematic representation of SHANK3-mediated inhibition of MAPK signaling in KRAS-mutant cancer cells.

b. The TCGA database analyses of SHANK3 mRNA expression in tumor vs. normal tissue. The dashed line indicates highly KRAS-mutated cancer types.

FIG. 7. SHANK3 silencing inhibits cell proliferation and growth of cancer cells harboring distinct KRAS mutations.

a-c. Representative western blot of SHANK3-silencing efficiency in protein level (upper panel) and relative proliferation (lower panel) of control (siCTRL) or SHANK3-silenced (siSHANK3_2 or siSHANK3_7) PANC-1 (KRASG12D mutant) (a), A549 (KRASG12S mutant) (b) and BxPC-3 (KRAS WT) (c) cells monitored in real-time using Incucyte Live-Cell Analysis system. Proliferation was graphed based on confluence.

d,e. Representative images (upper panel) and quantification (lower panels) of colony survival assay of control or SHANK3-silenced PANC-1 (d) and A549 (e) cells. Colony survival was graphed based on relative colony area and relative colony intensity. Data represent mean±s.d. Statistical analysis: unpaired two-tailed Student's t-test.

f, g. Representative images (f) and quantification (g) of control or SHANK3-silenced PANC-1 cells grown in 3D matrigel culture and monitored in realtime using Incucyte Live-Cell Analysis system. Organoid formation was graphed based on average area covered by organoids per image.

h. Quantification of proliferation of control or SHANK3-silenced KRAS-mutant PDAC (Panc10.05, KRASG12D; AsPC-1, KRASG12D; YAPC, KRASG12V; SW1990 KRASG12D; Su86.86, KRASG12D; Patu8902, KRASG12V), LUAD (H441; KRASG12V) and CRC (SW620, KRASG12V; HCT-115, KRASG13D; HCT-116, KRASG13D) cells, and KRAS WT cancer cells (HT-29, H292 and H226) monitored in real-time using Incucyte Live-Cell Analysis system. Shown is relative proliferation 4 days after silencing. PDAC, pancreatic adenocarcinoma; LUAD, lung adenocarcinoma; CRC, colorectal cancer.

FIG. 8. SHANK3 depletion triggers hyperactivation of KRAS-ERK signalling resulting in cell death in cells containing an oncogenic KRAS mutation

a. A scheme explaining effector-recruitment FRET analysis.

b. Effector-recruitment FRET analysis in HEK293 cells transiently co-expressing GFP-tagged KRAS4BG12V and mRFP-tagged C-RAF-RBD upon silencing of SHANK3. KRAS^{G12V}-RBD-recruitment was graphed based on relative FRET efficiency. Data represent mean±s.d. Statistical analysis: unpaired two-tailed Student's t-test.

c-h. Representative western blot (c-e) and quantification (f-h) showing levels of ERK1/2 phosphorylation (phospho-ERK1/2 (Thr202/Y204) relative to total ERK) and AKT phosphorylation (AKTS473 relative to total AKT) and PARP1 cleavage in control or SHANK3-silenced PANC-1 (c, f-h), A549 (d, f-h) and BxPC-3 (e, f-h) cells. Shown are the same samples as in FIG. 4a-c. Data represent mean±s.d. Statistical analysis: unpaired two-tailed Student's t-test (f-h).

i-j. Representative dot blot (i) and quantification (j) of AnnexinV/PI flow cytometry analysis of control or SHANK3-silenced PANC-1 cells analysed two days after silencing.

k-l. Representative images (k) and quantification (1) of control or SHANK3-silenced PANC-1 cells grown in 3D matrigel culture, stained by AnnexinV and monitored in real-time using Incucyte Live-Cell Analysis system. Apoptosis was graphed based on relative AnnexinV area within cells/organoids.

m. Proliferation of control (solid line) or SHANK3-silenced (dashed line) PANC-1 cells treated with DMSO or MEK/ERK inhibitors (Trametinib, Selumetinib, SCH772984) and monitored in real-time using Incucyte Live-Cell Analysis system. Shown is confluency (%) at day 5 after silencing/treatment.

FIG. 9. Schematic representation of SHANK3-mediated inhibition of RAS to target RAS-driven cancer through hyperactivation induced cell death.

FIG. 10. Loss of SHANK3 impairs growth of KRAS-mutant tumors in vivo

a-i, Tumor growth of control or SHANK3-silenced PANC-1 (a-c), A549 (d-f) and BxPC-3 (g-i) cells on CAM membranes. Shown are immunoblots of SHANK3 and GAPDH (loading control) (a, d, g; right panel) and tumor weight (a, d, g; left panel). Representative images (b, e, h) and quantification (c, f, i) of Ki-67 analyzed by IHC staining in tumor tissues at the end of experiments is shown. Data represent mean±s.d.; a, n=17 (siCTRL) and 23 (siSHANK3_7); d, n=27 (siCTRL) and 22 (siSHANK3_7); g, n=20 (siCTRL) and 19 (siSHANK3_7); n=10 tumors per sample group; unpaired Student's t-test with Welch's correction.

j, k, Representative images (j) and quantification (k) of cleaved caspase-3 analyzed by IHC staining in A549 tumor tissue from (d-f). Data represent mean±s.d.; n=10 tumors from 2 independent experiment; Mann Whitney test.

l, Representative images of cleaved caspase-3 staining in residual PANC-1 tumor tissue from (a-c).

m, Analysis of tumour growth of subcutaneously injected PANC-1 cells, with transient SHANK3 (siSHANK3_7) or scramble (siCTRL) silencing, at day 20 in nude mice. Shown SHANK3 mRNA levels were analysed to validate SHANK3 silencing. Data represent mean±s.d.; n=8 tumors per sample group; unpaired Student's t-test with Welch's correction.

n, o, Representative images (n) and quantification (o) of cancer cell number in HE-stained tumor samples from (m). Data represent mean±s.d.; n=8 tumors per sample group; unpaired Student's t-test with Welch's correction.

p, Representative images of subcutaneously injected PANC-1 tumors, with transient SHANK3 (siSHANK3_7) or scramble (siCTRL) silencing, at day 20 in nude mice.

FIG. 11. Doxycycline-induced loss of SHANK3 significantly suppresses the growth of established subcutaneous PANC-1 tumors.

a, b, SHANK3 gene expression (mRNA levels) showing the efficiency of SHANK3 silencing in control (−dox) or doxycycline-induced (+dox) (72 h) shSHANK3-expressing PANC-1 clones (shown are clones 1C and 4S).

c, d, Representative immunoblots showing the levels of pERK and cleaved-PARP1 in control or doxycycline-induced shSHANK3-expressing PANC-1 single-cell clones collected three days after induction (c) or at various time points (d). Samples were resolved and blotted on duplicate membranes (m#1 and m#2). GAPDH serves as a loading control. Cleaved-PARP1, indicative of apoptosis. ERK1/2 phosphorylation (phospho-ERK1/2 (Thr202/Y204)/total ERK), indicative of ERK activation.

e, f. Inducible silencing of SHANK3 inhibits proliferation in 2D. Quantification of cell proliferation in control or doxycycline-induced shSHANK3-expressing PANC-1 clones; doxycycline induction (+dox, 1 μg/ml) was started 24 hours post plating. Data represent mean±s.d.; 8 measurements; unpaired Student's t-test with Welch's correction.

g-i, Loss of SHANK3 in established PDAC spheroids impairs tumorigenic growth via hyperactivation-induced cell death. (g) Analysis of spheroid growth in a doxycycline-inducible SHANK3 shRNA KRAS-mutant PDAC cell model (PANC-1 cells, clone 1C). Spheroids were grown in 3D Matrigel. SHANK3-depletion was induced by doxycycline (+dox) at day 5 in established spheroids. Data represent mean±s.d., n=6 measurements from two independent experiment; Mann Whitney test. (h and i) Representative images (day 15; 10 days after dox-induction) and analysis of apoptosis (AnnexinV positive area) in established control (−dox) or SHANK3-depleted (+dox) PANC-1 spheroids. Both doxycycline and AnnexinV were added to spheroids at day 5. Data represent mean±s.d., C, n=6 (−dox) and 4 (+dox) measurements from two independent experiment; Mann Whitney test.

j. Schematic of doxycycline-inducible depletion of SHANK3 from established subcutaneous tumors.

k-m. (k) Growth rate of subcutaneously injected PANC-1 cell xenografts (5×10⁶ cells) with doxycycline-inducible (+dox) SHANK3 knockdown over the indicated time. Tumor growth was monitored with bi-weekly palpations. The arrow indicates the date when doxycycline treatment was initiated. (l) Tumor weights at the end of the experiment. (m) Tumor volumes after starting Dox-treatment normalised to tumor volumes at the start of the shSHANK3 induction. Dox: doxycycline-diet; control: normal diet. Data represent mean±s.d.; n=11 (dox) and 12 (control) tumors per sample group; unpaired Student's t-test with Welch's correction.

FIG. 12. Targeting SHANK3 to induce RAS hyperactivation-induced apoptosis represents a conceptually novel therapeutic approach for the treatment of KRAS-driven/dependent cancers.

Schematic representation of SHANK3-controlled cell fate in KRAS mutant/driven/dependent cancers. SHANK3 is an endogenous modulator of KRAS that sustains oncogenic RAS-ERK signalling at an optimal level—below toxic oncogenic signaling—in KRAS mutant cancers. Loss of endogenous SHANK3 drives KRAS-mutant cells to ERK hyperactivation-induced cell death.

FIG. 13. Sequences:
a. Human SHANK3 protein (SEQ ID NO: 1) including SPN (SEQ ID NO: 11) domain.
b. Rat Shank3 protein (SEQ ID NO: 2) including SPN (SEQ ID NO: 12) domain.
c. Mouse Shank3 (SEQ ID NO: 3) protein including SPN (SEQ ID NO: 13) domain.
d. Alignment of human, rat and mouse SHANK3 protein sequences (SEQ ID NOs: 1, 2 and 3).
e. Human SPN domain (SEQ ID NO: 11).
f. Rat SPN domain (SEQ ID NO: 12).
g. Mouse SPN domain (SEQ ID NO: 13).
h. Human SHANK3 siRNA #2 target-specific region (SEQ ID NO: 4)
i. Human SHANK3 siRNA #7 target-specific region (SEQ ID NO: 5)
j. Human HRAS isoform 1, also known as H-Ras4A, p21 (SEQ ID NO: 6)
k. Human HRAS isoform 2, also known as: H-RasIDX, p19 (SEQ ID NO: 7)
l. Human KRAS isoform 1, also known as K-Ras4A (SEQ ID NO: 8)
m. Human KRAS isoform 2, also known as K-Ras4B (SEQ ID NO: 9)
n. Human NRAS (SEQ ID NO: 10)
o. Human SHANK3 shRNA target-specific region (SEQ ID NO:17)

Sequences of SEQ ID NO: 14, 15, 16, 18-37 are included in attached the Sequence Listing only.

FIG. 14. The RAS Pathway (simplified model). Genes highlighted in pink are frequently deleted in human cancers and RASopathies. Genes in green are frequently activated by mutation

### DETAILED DESCRIPTION OF THE INVENTION

More than a quarter of all cancers are driven by mutations in the RAS family of genes. Considering the key role of these oncogenes, and despite intensive effort, no effective anti-RAS strategies have successfully made it to the clinic. The inventors surprisingly found that a class of neuronal scaffold proteins—SHANK family proteins—expressed in cancer, bind to active/mutated forms of RAS proteins to moderate RAS signaling. Accordingly, it was surprisingly shown that loss of one of the scaffolding protein isoforms—SHANK3—in RAS-mutant cancers triggers RAS signaling activation, tipping the balance from proliferation to cytotoxic signaling and leading to cell death. As such, drugging this scaffold protein i) represents a completely innovative approach to target RAS-driven cancers that exploits, rather than counters, the oncogene's function, and ii) delivers an alternative cancer treatment for patients that do not respond to current standards of care.

In some embodiments, human SHANK3 comprises or consists of a polypeptide having an amino acid sequence set forth in SEQ ID NO: 1. In some embodiments human SHANK3 comprises or consists of a polypeptide having an amino acid sequence set forth in SEQ ID NO: 36 which includes 75 additional N-terminal amino acids as compared to the SHANK3 polypeptide of SEQ ID NO: 1.

In the present invention evidence is provided that SHANK3 SPN directly interacts with oncogenic RAS, most preferably KRAS, and limits its downstream signaling to RAF-MEK-ERK pathway to inhibit tumour growth. Thus, SHANK3 is an endogenous inhibitor of RAS. Importantly, it was found that SHANK3 is expressed at low levels in RAS-mutant cancer cells and its depletion triggers activation of RAS/MAPK/ERK signalling resulting in growth arrest or apoptosis in cells harbouring an oncogenic RAS mutation.

In particular, the present invention relates to the finding that blocking RAS inhibition promotes activation of downstream signaling and cytotoxicity in RAS-driven cancers. Activation of RAS signaling may be understood as increased function of RAS. Preferably, the activation of RAS signaling may be at least 10%, preferably at least 15%, more preferably 30% or most preferably at least 50%, increased as compared to the RAS signaling in a situation wherein RAS inhibition by SHANK3 is not blocked. An increase in the activation of RAS signaling may be assessed by any method suitable to investigate RAS downstream signaling including but not limited to measurement of ERK activity. Means and methods to this end are readily available in the art.

In an earlier large-scale RNAi screen, SHANK3 was identified as a novel integrin inhibitor. Interestingly, when the crystal structure of a SHANK3 N-terminal fragment was resolved, it was found that the SHANK3 N-terminal (SPN) domain contained a RAS-binding structure. Further analyses revealed that the SHANK3 SPN domain binds specifically to active RAS- and Rap-family GTPases. The sequence of SPN domain is shown in SEQ ID NOs: 11, 12 and 13. Human SPN domain corresponds to amino acids 1-93 of SEQ ID NO: 1 and to amino acids 76-168 of SEQ ID NO: 36. The present invention shows that SHANK3-SPN binds directly to active KRAS and limits downstream signaling through the RAF-MEK-ERK pathway. Thus, the present invention shows that SHANK3 is a novel endogenous inhibitor of RAS. Importantly, it was found that SHANK3 remains expressed at low levels in RASmutant cancer cells—perhaps to maintain ERK activity below the cytotoxic threshold—and its depletion triggers enhanced ERK activation resulting in growth arrest or apoptosis in KRAS cancer models.

Thus, in the present invention, based on the SHANK3 N-terminal crystal structure, the binding interface between SHANK3 and RAS, identifying critical residues for active KRAS binding and subsequent inhibition was determined. The present invention shows that mutation of these residues abolishes KRAS binding, confirming the interaction interface on RAS. It also shows that SHANK3 knockdown/silencing triggers activation of the RAS signaling cascade and cell death. Furthermore, the present invention relates to SHANK3 knockdown/silencing inhibition of tumor growth, e.g. in vitro and in ovo. It has to be noted that effects of SHANK3 inhibition and/or knockdown (depletion, loss of function) are not restricted to RAS-mutant cancers but apply also to wild-type RAS cancers, provided that the wild-type RAS cancers are RAS-dependent, i.e. driven by RAS activation e.g. through other genetic alterations.

Thus, the present invention indicates that targeting RAS-SHANK3 association is a highly efficacious therapeutic avenue for RAS-driven cancer patients.

Importantly, the present invention shows that inducible depletion of SHANK3 dramatically impairs the growth of established PDAC tumors in vivo. These results indicate that an inducible depletion of endogenous SHANK3 is effective in blocking KRAS-mutant tumor growth in vivo.

The three RAS genes (HRAS, KRAS, and NRAS), harbouring activating mutations, comprise the most frequently mutated oncogene family in cancer (27%; Catalogue of Somatic Mutations in Cancer [COSMIC] v80). KRAS is the predominant or exclusive RAS gene mutated in three of the top four neoplasms that account for cancer deaths in the US and in Europe: pancreatic ductal adenocarcinoma (PDAC), lung adenocarcinoma (LUAD) and colorectal cancer (CRC). The mutations render KRAS persistently GTP-bound (constitutively active) independent of extracellular stimuli, resulting in stimulation of effector signalling pathways to drive cancer growth. Moreover, KRAS activation is one of the signalling pathways involved in resistance to EGFR tyrosine kinase inhibitors (TKIs) and monoclonal antibodies. In this context, despite EGFR inhibition, EGF-mediated downstream signalling is maintained through KRAS activation. Thus, an effective anti-RAS therapy will have a significant impact on the inhibition of cancer growth and on improving patient survival.

“Diminishing the amount of RAS-dependent cancer cells” relates to decreasing the number RAS-dependent i.e. RAS-driven cancer or tumour cells.

Persons skilled in the art understand what is meant by the RAS-pathway and the RAS signalling. In FIG. 14 a schematic example drawing of the RAS-pathway is shown. It is to be noted that RAS signalling can be activated by a number of cellular receptors including receptor tyrosine kinases (RTKs), G-protein coupled receptors (GPCRs), and integrin family members as well as Ras guanine nucleotide exchange factors (Ras-GEFs). Thus, proteins, which activate RAS-pathway may include but are not limited to EGFR, RTK family members, GPCRs, integrins and RAS-GEFs.

Epidermal growth factor receptor (EGFR) is a member of the RTK family and one of the best characterized activators of RAS signalling through recruitment of the molecular scaffolding protein growth factor receptor bound protein 2 (GRB2). GRB2 recruits the RAS-guanine exchange factor (RAS-GEF) SOS1, which activates the RAS protein through a conformational change induced by exchanging GDP for GTP.

Similarly, other RTK family members including platelet derived growth factor receptor beta (PDGFR-β) can initiate RAS activation through recruitment of GRB2, and colony stimulating factor 1 receptor (CSF-1R) signaling functions through activation of RAS.

Several GPCRs also function in a RAS-dependent manner with the betagamma subunit of GPCRs activating RAS signaling. GPCRs activate RAS through stimulation of both non-RTKs (including src, Lyn, and Syk) and RTKs as described above.

Certain downstream signaling functions of integrin proteins are also RAS dependent.

RAS signaling can be further activated by additional RAS-GEFs including the RAS-GRF and RAS-GRP family members or negatively modulated by a series of RAS-GTPase activating enzymes (RAS-GAPs), including neurofibromin 1 (NF1). These RAS pathway activity regulators are also frequently altered across a number of cancer types

The aberrant RAS signaling in tumors can be contributed by several different mutations, mostly activating mutation in tumor cells: in K-RAS, N-RAS or H-RAS.

The activating mutations mostly affect the GTPase activity of RAS leading to accumulation of RAS-bound GTP. These GTP-bound RAS can activate other downstream effector proteins leading to constitutive abnormal signaling and anarchy within the tumor cell. The impaired ability of Ras mutants to hydrolyze GTP, either intrinsically or in response to GAPs, is responsible for the oncogenic nature of mutations at residues G12, G13, and Q61 in the active site.

Ras remains activated due to loss of GAP-accelerated GTP hydrolysis. One such typical example of GAP mutation is the GAPs, neurofibromin encoded by the NF1 tumor suppressor gene. Patients with neurofibromatosis type I inherit only one functional NF1 gene and then predisposed to cancer through complete loss of NF1.

Ras signaling has also been known to be activated in tumors in which growth factor receptor tyrosine kinase has been overexpressed. The most common example are epidermal growth factor receptor (EGFR) and receptor tyrosine-protein kinase erbB-2 (ERBB2) which are activated and overexpressed in many types of cancer including breast, ovarian, and stomach carcinomas.

More than 30 percent of all human cancers—including 95 percent of pancreatic cancers and 45 percent of colorectal cancers—are driven by or dependent on mutations of the RAS family of genes.

Although the specificity between tumor type and mutated Ras oncogene is not absolute, K-ras mutations are more frequently found in adenocarcinomas and solid tumors, whereas N-ras is the prevalent Ras gene mutated in leukemias, thyroid carcinomas, or malignant melanoma and H-ras mutations are sparingly found, with a prevalence in bladder carcinoma and low incidence cancers such as seminomas or Hurthle cell carcinomas.

In the following a few non-limiting examples of RAS-driven cancers are indicated.

Pancreatic Ductal Adenocarcinoma

Pancreatic adenocarcinomas are among the most aggressive and with worst prognosis and outcome in humans. These tumors display the highest reported incidence of ras mutations among all human cancers, almost exclusively on the K-Ras locus.

Colorectal Carcinoma

K-ras mutations are common events detected in 40-45% of all colorectal carcinoma, but lower mutation rates have been found in N-ras.

Non-Small Cell Lung Carcinoma

Non-small cell lung carcinomas (NSCLCs) harbour a high frequency of K-ras mutations and low rates of oncogenic changes in either N-ras or H-ras.

Malignant Melanoma

Together with bladder carcinomas, melanomas are the only high-incidence/high-mortality solid tumors in humans in which K-ras mutations are not prevalent over N-ras or H-ras mutations. Specifically, N-ras mutations are found in 20-30% of malignant melanoma samples analyzed.

Urinary Bladder Carcinoma

The rates of H-ras mutations detected in human bladder carcinomas are ranging from none to 30% of all bladder carcinomas analyzed. Despite the mediumlow ras mutation levels detected, the crucial role of Ras proteins in bladder cancer has been highlighted.

Thyroid Carcinomas

Mutations in RAS genes occur, on average, in 30-45% follicular thyroid cancer (FTC), 30-45% follicular variant papillary thyroid cancer (FVPTC), 20-40% poorly differentiated thyroid cancer (PDTC), 10-20% anaplastic thyroid cancer, and rarely classical papillary thyroid cancer (PTC). RAS mutations also occur in 20-25% benign follicular thyroid adenoma (FTA).

Hematopoietic Malignancies

Mutations in NRAS, KRAS, and the NF1 tumor suppressor, which encodes a GAP called neurofibromin, are strongly associated with myeloid malignancies.

Ras Mutation in Other Tumor Types

Ras mutations are more uncommon in other high-incidence cancers but do exist such as prostate, breast, or liver carcinomas. The prevalence of RAS mutation in breast cancer is between 7% and 12%. In hepatocellular carcinomas, where RAS mutations are found in less than 10% of tumors, it has been shown that WT Ras proteins become activated through a mechanism involving the inactivation of Ras-GAPs. Neuroblastomas, cervix adenocarcinomas, or stomach cancers also harbor low rates of RAS mutation. In addition, significant frequencies of KRAS mutations locus are detected in some lower incidence cancers such as biliary tract adenocarcinomas (35%), angiosarcomas (49%), or malignant fibrous histiocytoma (16%), where H-ras mutations have also been found (15%). Moreover, HRAS and NRAs mutations have been found in neck and head cancer.

Thus, the present invention, i.e. blocking RAS inhibition to promote activation of downstream signalling and cytotoxicity in RAS-driven cancers provides surprising new insights into cancer therapy.

Indeed, KRAS is the most commonly mutated gene, in 86% of RAS-driven cancers including pancreatic, lung and colorectal cancer. There is significant and compelling evidence that KRAS represents the initiating event in lung and pancreatic adenocarcinomas, two cancer types with some of the worse prognoses.

The RAS-RAF-MEK-ERK pathway (ERK signalling) is essential for KRAS-induced cell transformation and especially drives the growth of KRAS-mutant PDAC.². While ERK activation generally stimulates growth and survival programmes, excessive ERK activation can instead cause growth arrest^{7,8} or apoptosis⁹. Thus, tumors with mutant oncogenes in the RAS pathway must restrain the activity of ERK1/2 to avoid toxicities and enable tumor growthll. This vulnerability to extensive ERK activation raises the possibility of novel therapeutic approaches for RAS-mutant cancers.

SHANK family proteins (SH3 and multiple ankyrin repeat domains, SHANK, 1-3) are a family of scaffold proteins that the inventors identified SHANK3 as inhibitors of major cell adhesion receptors, integrins¹³. In this study, they found that the N-terminal SPN domain of SHANK3 protein is an unexpected Ras-association (RA) domain with high affinity for active (GTP-bound) Rap- and Ras-family GTPases. Surprisingly, the inventors found that SHANK3, preferably, SPN domain of SHANK, directly interacts with oncogenic RAS and limits its downstream signalling to RAF-MEK-ERK pathway to inhibit tumour growth.

Thus, the present invention provides a means for cancer therapy by targeting SHANK3, for example by depleting the SHANK3 protein with RNAi, degrader technologies or other means, or by blocking the SHANK3-RAS interaction thereby triggering cell death in RAS-driven cancers.

As used herein, the term “SHANK3 inhibiting agent” refers to any agent that blocks the SHANK3 function. Preferably, a SHANK3 inhibiting agent silences or down-regulates the expression of SHANK3 gene, edits SHANK3 by targeted gene disruption, or blocks or interferes with the function of SHANK3 as a RAS inhibitor.

As used herein, the term “SHANK3 silencing” refers to complete or partial reduction of SHANK3 gene expression. In some embodiments, SHANK3 gene expression is reduced e.g. by at least 50%, or at least 80%, 81%, 82%, 83%, 84%, 85%, 86%, 87%, 88%, 89%, 90%, 91%, 92%, 93%, 94%, 95%, 96%, 97%, 98%, 99%, or 100% when a SHANK3 silencing agent is introduced into a human or animal subject.

The loss of SHANK protein or loss of SHANK3 transcript may be obtained by any suitable method or means known in the art including, but not limited to, RNA interference (RNAi), gene editing and ribozymes that cleave the SHANK3 mRNA. The ribozyme technology is described, for example, by Li et al. in Adv. Cancer Res., 2007, 96:103-43¹⁶. Methods triggering loss of protein are well known to those skilled in the art and include, but are not limited to, Protac.

The most common approach for RNAi-based gene silencing is the use of small interfering RNA (siRNA). The principle of siRNA is extensively presented in literature. As examples can be mentioned the US patent publications 2003/0143732, 2003/0148507, 2003/0175950, 2003/0190635, 2004/0019001, 2005/0008617 and 2005/0043266. An siRNA duplex molecule comprises an anti-sense region and a sense strand wherein said antisense strand comprises nucleotide sequence complementary to a target region in an mRNA sequence encoding a certain protein, and the sense strand comprises nucleotide sequence complementary to the said antisense strand. In other words, siRNAs are small double-stranded RNAs (dsRNAs). The sense strand and antisense strand can be covalently connected via a linker molecule, which can be a polynucleotide linker or a non-nucleotide linker. The length of the antisense and sense strands may vary and is typically about 19 to 21 nucleotides each. In some cases, the siRNA may comprise 22, 23 or 24 nucleotides. siRNA molecules which have been used in the working examples comprise SEQ ID NO: 4 or SEQ ID NO: 5, depicted in FIGS. 13h and 13i, respectively. To be more specific, said siRNA molecules comprise a target-specific antisense region having a nucleic acid sequence set forth in SEQ ID NO: 4 or SEQ ID NO: 5. It is also envisaged that siRNA molecules comprising a target-specific region having a nucleic acid sequence set forth in any one of SEQ ID Nos: 17-35 are suitable for silencing SHANK3. In further embodiments, the target-specific antisense region may comprise or consist of a nucleic acid sequence having at least 80% identity, preferably at least 85% identity, more preferably at least 90% identity, more preferably 95% identity, more preferably 98% identity, more preferably 99% identity to the sequence set forth in any one of SEQ ID NOs: 4, 5 and 17-35, provided that the siRNA's ability to inhibit SHANK3 gene expression as compared to a siRNA whose target-specific region corresponds to SEQ ID NO: 4, 5 or 17-35 is retained.

Another approach for RNAi-based SHANK3 silencing is to use longer, typically 25-35 nt, Dicer substrate siRNAs (DsiRNAs), which in some cases have been reported to be more potent than corresponding conventional 21-mer siRNAs¹⁶. DsiRNAs are processed in vivo into active siRNAs by Dicer. In a cell, an active siRNA antisense strand is formed and it recognizes a target region of the target mRNA. This in turn leads to cleaving of the target RNA by the RISC endonuclease complex (RISC=RNA-induced silencing complex) and also in the synthesis of additional RNA by RNA dependent RNA polymerase (RdRP), which can activate Dicer and result in generation of additional siRNA duplex molecules, thereby amplifying the response.

As used herein, the term “small double-stranded RNA” (dsRNA) refers to both siRNAs and DsiRNAs.

Typically, but not necessarily, the antisense strand and the sense strand of dsRNA both comprise a 3′-terminal overhang of a few, typically 1 to 3 nucleotides. The 3′ overhang may include one or more modified nucleotides, such as a 2′-O-methyl ribonucleotide. The 5′-terminal of the antisense is typically a phosphate group (P). The dsRNA duplexes having terminal phosphate groups (P) are easier to administrate into the cell than a single stranded antisense. In some cases, the 5′-terminal of the sense strand or of both antisense and sense strands may comprise a P group.

Artificial microRNA (miRNA) precursors are another class of small RNAs suitable for mediating RNAi. Typically, artificial miRNA precursors are about 21-25 nucleotides in length, and they may have 1 to 3, typically 2, overhanging 3′ nucleotides.

Short-hairpin RNAs (shRNAs) are still another way of silencing SHANK3 by RNAi. shRNAs consist of i) a short nucleotide sequence, typically ranging from 19 to 29 nucleotides, derived from the target gene; ii) a loop, typically ranging between 4 to 23 nucleotides; and iii) a short nucleotide sequence reversely complementary to the initial target sequence, typically ranging from 19 to 29 nucleotides. In a preferred embodiment, the shRNA molecule comprises a target-specific region having a nucleic acid sequence set forth in SEQ ID NO: 17, depicted in FIG. 13o. It is also envisaged that shRNA molecules comprising a target-specific region having a nucleic acid sequence set forth in any one of SEQ ID Nos: 4, 5 and 18-35 are suitable for silencing SHANK3. In further embodiments, the target-specific antisense region may comprise or consist of a nucleic acid sequence having at least 80% identity, preferably at least 85% identity, more preferably at least 90% identity, more preferably 95% identity, more preferably 98% identity, more preferably 99% identity to a sequence set forth in any one of SEQ ID NOs: 4, 5 and 17-85, provided that the shRNA's ability to inhibit SHANK3 gene expression as compared to a shRNA whose target-specific region corresponds to SEQ ID NO: 4, 5 or 17-35 is retained.

SHANK3 silencing may also be obtained by antisense therapy, where relatively short (typically 13-25 nucleotides) synthetic single-stranded DNA or RNA oligonucleotides inactivate SHANK3 gene by binding to a corresponding mRNA. Antisense oligonucleotides may be unmodified or chemically modified. In some embodiments, the hydrogen at the 2′-position of ribose is replaced by an O-alkyl group, such as methyl. In further embodiments, antisense oligonucleotides may contain one or more synthetic or natural nucleotide analogs including, but not limited to peptide-nucleic acids (PNAs).

Delivery of SHANK3 specific RNAi molecules can be accomplished in two principally different ways: 1) endogenous transcription of a nucleic acid sequence encoding the oligonucleotide, where the nucleic acid sequence is located in an expression construct, or 2) exogenous delivery of the oligonucleotide.

For endogenous transcription, SHANK3 specific RNAi molecules may be inserted into suitable expression systems using methods known in the art. Non-limiting examples of such expression systems include retroviral vectors, adenoviral vectors, lentiviral vectors, other viral vectors, expression cassettes, and plasmids, such as those encapsulated in pegylated immunoliposomes (PILs), with or without one or more inducible promoters known in the art. If dsRNA is employed, both RNA strands may be expressed in a single expression construct from the same or separate promoters, or the strands may be expressed in separate expression constructs.

Typically, expression constructs are formulated into pharmaceutical compositions prior to administration to a human or animal subject. Administration may be performed by any suitable method known in the art, including systemic and local delivery. The formulation depends on the intended route of administration as known to a person skilled in the art. By way of example, the expression construct may be delivered in a pharmaceutically acceptable carrier or diluent, or it may be embedded in a suitable slow release composition. In some cases, the pharmaceutical composition may contain one or more cells producing the expression construct. Also bacteria may be used for RNAi delivery. For instance, recombinantly engineered Escherichia coli can enter mammalian cells after in vivo delivery and transfer shRNAs. A related approach is to use minicells derived e.g. from Salmonella enterica.

For exogenous delivery, RNAi molecules are typically complexed with liposome or lipid-based carriers, cholesterol conjugates, or polyethyleneimine (PEI). A promising new approach is to complex dsRNAs with stable nucleic acid lipid particles (SNALPs). Suitable routes of administration for exogenous delivery, with or without said complexing, include, but are not limited to, parenteral delivery (e.g. intravenous injection), enteral delivery (e.g. orally), local administration, topical administration (.e.g. dermally or transdermally) as known to a person skilled in the art. Since surgical removal of a tumour is usually the primary clinical inters vention, RNAi molecules may be administered directly to the resected tumour cavity.

Normal, unmodified RNA has low stability under physiological conditions because of its degradation by ribonuclease enzymes present in the living cell or biological fluid. If the oligonucleotide shall be administered exogenously, it is highly desirable to modify the molecule according to known methods so as to enhance its stability against chemical and enzymatic degradation.

Modifications of nucleotides to be administered exogenously in vivo are extensively described in the art (e.g. in US 2005/0255487, incorporated herein by reference). Principally, any part of the nucleotide, i.e. the ribose sugar, the base and/or internucleotidic phosphodiester strands can be modified. For example, removal of the 2′-OH group from the ribose unit to give 2′-deoxyribosenucleotides results in improved stability. Prior disclosed are also other modifications at this group: the replacement of the ribose 2′-OH group with alkyl, alkenyl, allyl, alkoxyalkyl, halo, amino, azido or sulfhydryl groups. Also other modifications at the ribose unit can be performed: locked nucleic acids (LNA) containing methylene linkages between the 2′- and 4′-positions of the ribose can be employed to create higher intrinsic stability.

Furthermore, the internucleotidic phosphodiester linkage can, for example, be modified so that one or more oxygen is replaced by sulfur, amino, alkyl or alkoxy groups. Also the base in the nucleotides can be modified.

Preferably, the oligonucleotide comprises modifications of one or more 2′-hydroxyl groups at ribose sugars, and/or modifications in one or more internu-cleotidic phosphodiester linkages, and/or one or more locked nucleic acid (LNA) modification between the 2′- and 4′-position of the ribose sugars.

Particularly preferable modifications are, for example, replacement of one or more of the 2′-OH groups by 2′-deoxy, 2′-O-methyl, 2′-halo, e.g. fluoro or 2′-methoxyethyl. Especially preferred are oligonucleotides where some of the internucleotide phoshodiester linkages also are modified, e.g. replaced by phosphorothioate linkages.

In some embodiments, RNAi molecules may contain one or more synthetic or natural nucleotide analogs including, but not limited to, phosphorothioates, phosphoramidates, methyl phosphonates, chiral-methyl phosphonates, and peptide-nucleic acids (PNAs) as long as dsRNAs retain their SHANK3 silencing ability.

It should be stressed that the modifications mentioned above are only non-limiting examples.

One of the challenges related to RNAi is the identification of a potent RNAi molecule for the corresponding mRNA. It should be noted that genes with incomplete complementarity are inadvertently downregulated by the RNAi, leading to problems in data interpretation and potential toxicity. This however can be partly addressed by carefully designing appropriate RNAi molecules with design algorithms. These computer programs sieve out given target sequence with a set of rules to find sequence stretches with low GC content, a lack of internal repeats, an A/U rich 5-end and high local free binding energy which are features that enhance the silencing effect of dsRNA.

In order to identify agents useful in the present invention, SHANK3 silencing RNAi molecules can be designed by using commercial or non-commercial algorithms available in the art. This may be achieved e.g. by loading the full length cDNA sequence of SHANK3 to an algorithm program. In one embodiment, the nucleic acid sequence set forth in SEQ ID NO: 14 represents the cDNA sequence of SHANK3 mRNA. In another embodiment, the nucleic acid sequence set forth in SEQ ID NO: 37 represents the cDNA sequence of SHANK3 mRNA, recently updated to contain 346 additional nucleotides at the 5′ end as compared to SEQ ID NO: 14. Algorithm-generated RNAi sequences can then screened trough genome wide DNA sequence alignment (BLAST) to eliminate RNAi molecules which are not free from off-targeting. In other words, all those RNAi molecules which have even short sequence regions matching with other genes than target gene (SHANK3) may be considered invaluable for further use. Non-limiting examples of algorithm programs suitable for designing siRNAs include Eurofins MWG Operon's Online Design Tool or a stand-alone program developed by Cuia et al.¹⁷. Algorithm programs suitable for designing other types of RNAi molecules, such as shRNA and miRNA molecules, are also readily available in the art.

Obtained RNAi molecules can then be synthetized and transfected to different cell lines and their capacity to degrade mRNA and further deplete translation of SHANK3 can be studied at protein level by measuring the amount of SHANK3 protein after siRNA treatment with SHANK3 specific antibodies or by analysing mRNA levels of SHANK3 with sequencing or q-RT-PCR.

Suitable SHANK3 specific RNAi sequences suitable for use in various embodiments of the present invention can be designed and synthetized according to methods known in the art. Any such isolated RNAi sequence must be sufficiently complementary to SHANK3 mRNA sequence in order to silence SHANK3 gene but lack significant off-targeting. This means that although 100% complementarity is preferred, also RNAi sequences with lower complementarity may be suitable for use in the present invention. Those skilled in the art are able to determine the required complementarity for each case.

The term “complementary” is well known in the art and it means Watson-Crick base pairing where nucleobase adenine (A) in a target motif sequence is represented by nucleobase thymine (T) in a corresponding binding unit, or vice versa. Accordingly, nucleobase cytosine (C) in a target motif is represented by nucleobase guanine (G) in a corresponding binding unit, or vice versa. In other words, the complementary sequence to, for instance, 5′-T-T-C-A-G-3′ is 3′-A-A-G-T-C-S′. As is readily understood by those skilled in the art, RNA differs from DNA by containing uracil (U) instead of T. Uracil is complementary to adenine.

Accordingly, although the most preferred siRNA and shRNA molecules may, at least in some embodiments, be those whose target-specific regions comprise or consist of polynucleotides having 100% sequence identity with any one of SEQ ID Nos: 4, 5 and 17-35, also siRNAs and shRNAs having lower sequence identity are envisaged. Accordingly, suitable siRNA and shRNA molecules include also those whose target-specific regions have e.g at least 20%, or at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity with SEQ ID NO:s 4, 5 and 17-35, as long as they have similar binding properties and SHANK3 silencing activity as the reference RNAi molecules. One aspect of the invention relates to such siRNA and shRNA molecules.

As used herein, the percent identity between two nucleic acid sequences is equivalent to the percent homology between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using standard methods known in the art.

In some embodiments, SHANK3 inhibition may be contemplated by a nuclease system comprising at last one genome targeted nuclease and at least one guide RNA comprising at least one targeted genomic sequence. Preferably, the nuclease system is Clustered Regularly Interspaced Short Palindromic Repeats (CRISPR) and CRISPR-associated endonuclease protein (cas) system, i.e. CRISPRCas system, preferably CRISPR-Cas9 system.

As used herein, the term “guide RNA” (gRNA) molecule refers to a short synthetic nucleic acid molecule that promotes the specific targeting or homing of a gRNA molecule/Cas molecule complex to a target nucleic acid. In other words, gRNA provides both targeting specificity and scaffolding/binding ability for Cas9 nuclease. To this end, gRNA is composed of a “scaffold” sequence necessary for Cas9-binding and a user-defined ˜20 nucleotide “targeting domain” which defines the genomic target to be modified. gRNA does not exist in nature.

In certain embodiments, the gRNA molecule may be a unimolecular or chimeric gRNA consisting of a single RNA molecule. In other embodiments, the gRNA molecule may be a modular gRNA comprising more than one, and typically two, separate RNA molecules.

The present gRNA molecules comprise a targeting domain that is complementary to a target sequence in the genomic DNA encoding human SHANK3. The targeting domain comprises a nucleotide sequence that is e.g., at least 80%, 85%, 86%, 87%, 88%, 89%, 90%, 91%, 92%, 93%, 94%, 95%, 96%, 97%, 98%, 99%, or 100% complementary to the target sequence on the target SHANK3 nucleic acid. In some embodiments, the targeting domain may be 5 to 50, 10 to 40, 10 to 30, 15 to 30, or 15 to 25 nucleotides in length. In some more specific embodiments, the targeting domain is 15, 16, 17, 18, 19, 20, 21, 22, 23, 24 or 25 nucleotides in length. Some or all of the nucleotides of the domain can have a modification.

In some embodiments, the targeting domain is configured to provide SHANK3 knockdown by introducing a frameshift mutation or a stop codon into the human genomic SHANK3 DNA.

gRNA targeting domain sequences suitable for knocking down SHANK3 gene may be designed. Such gRNA molecules induce potentially insertions or deletions in an area that encodes the very N-terminal part of SHANK3 protein, and lead to a frameshift resulting in impaired expression of SHANK3.

Further gRNA targeting domain sequences suitable for use in the present invention can be designed and analysed using software tools available in the art (e.g. the one available at http://crispr.mit.edu/). Such tools can be used to optimize the selection of gRNA within the target sequence, e.g., to minimize or predict total off-target activity across the genome. In other words, each possible gRNA can be ranked according to its total predicted off-target cleavage; the top-ranked gRNAs represent those that are likely to have the greatest on-target and the least off-target cleavage. Candidate gRNA molecules can then be validated in vitro and/or in vivo according to methods available in the art.

As used herein, the term “Cas” refers to a protein that can interact with a gRNA molecule and, in concert with the gRNA molecule, target or home to a site which comprises a target domain and a protospacer adjacent motif (PAM) sequence.

In some embodiments, the Cas protein is a Cas9 protein. As is well known in the art, Cas9 may be derived from or based on Cas9 proteins of a variety of species including, but not limited to, Streptococcus pyogenes, Streptococcus thermophilus, Staphylococcus aureus, and Neisseria meningitides. Modified Cas9 proteins with desired properties can be obtained by using any suitable means and methods available in the art.

As used herein, the term “protospacer adjacent motif” (PAM) is a sequence in the target nucleic acid. The Cas9 molecule interacts with the PAM sequence and cleaves the target nucleic acid upstream from the PAM sequence. Cas9 molecules from different bacterial species can recognize different PAM sequence motifs. For example, Streptococcus pyogenes Cas9 recognizes the sequence motif NGG, Streptococcus thermophiles Cas9 recognizes the sequence motif NGGNG and NNAGAAW (W=A or T), Staphylococcus aureus Cas9 recognizes the sequence motif NNGRR (R=A or G), whereas Neisseria meningitides Cas9 recognizes the sequence motif NNNNGATT. Cas9 directs cleavage of the target nucleic acid sequence about 20 base pairs upstream from the PAM. The ability of a Cas9 molecule to recognize a PAM sequence can be determined, e.g., using a transformation assay described in Jinek et al., Science 2012, 337:816¹⁸.

Naturally occurring Cas9 molecules can recognize specific PAM sequences as explained above. Thus, in some embodiments Cas9 molecules having the same PAM specificities as naturally occurring Cas9 molecules are employed. In other embodiments, Cas9 molecules having altered PAM specificities may be employed, for example to decrease the number of off target sites and/or to improve specificity. Those skilled in the art know how to obtain such non-natural Cas molecules.

As used herein, the term “donor template” or “template nucleic acid” refers to a nucleic acid sequence which can be used in conjunction with a Cas9 molecule and a gRNA molecule to alter the structure of a target position by participating in a homology-directed repair (HDR) event. In some embodiments, the target nucleic acid is modified to have some or all of the sequence of the template nucleic acid, typically at or near cleavage site(s). For use in the present invention, a preferred template nucleic acid provides a stop codon into the target site. In some embodiments, the template nucleic acid results in the incorporation of a modified or non-naturally occurring base into the target nucleic acid.

Cas9 nucleases to be employed in the present invention may differ in their DNA cleaving properties. In some embodiments, naturally occurring Cas9 molecules having a double stranded nuclease activity, i.e., the ability to cleave both strands of a double stranded nucleic acid and create a double stranded break, are employed. Double-stranded breaks activate the doublestrand break (DSB) repair machinery. DSBs can be repaired by the cellular Non-Homologous End Joining (NHEJ) pathway, resulting in insertions and/or deletions (indels) which disrupt the targeted locus. Alternatively, if a donor template with homology to the targeted locus is supplied, the DSB may be repaired by the homology-directed repair (HDR) pathway allowing for precise replacement mutations, such as ones creating a stop codon, to be made. Such embodiments require only a single gRNA.

In some other embodiments, mutant Cas9 molecules, such as Cas9D10A or Cas9H840A, having only nickase activity may be employed. Such Cas molecules cleave only one DNA strand resulting in a single nick that does not activate NHEJ. Instead, when provided with a homologous donor template, DNA repairs are conducted via the high-fidelity HDR pathway only, increasing the ratio of HDR to NHEJ at a given cleavage site. Thus, such embodiments are more suitable for creating stop codons through donor template instead of resulting in indels.

In some further embodiments, two mutated Cas9 molecules, such as those comprising either D10A or H840A mutation, having only nickase activity may be employed together with two gRNAs, one for placement of each single strand break. Such paired Cas9 complexes do not activate NHEJ but when provided with a homologous donor template, result in DNA repairs by HDR pathway only, resulting in reduced indel mutations. Thus, such embodiments are more suitable for creating stop codons through donor template instead of resulting in indels.

In some even further embodiments, a nuclease-deficient Cas9, such as Cas9 molecule comprising both H840A and DMA mutations, may be employed. Such Cas9 molecules do not have cleavage activity, but do have DNA binding activity. Therefore, such variants can be used to sequence-specifically target any region of the genome without cleavage. Instead, by fusing with various effector domains, nuclease-deficient Cas9 can be used as a gene silencing tool by means and methods known in the art.

While the above-mentioned embodiments involve either a single double-strand break or two single strand breaks, further embodiments may involve two double stranded breaks with a break occurring on each side of the target sequence, one double stranded breaks and two single strand breaks with the double strand break and two single strand breaks occurring on each side of the target sequence, or four single stranded breaks with a pair of single stranded breaks occurring on each side of the target sequence.

The ability of a Cas9 molecule/gRNA molecule complex to bind to and cleave a target nucleic acid can be evaluated by techniques available in the art including, but not limited to, a plasmid cleavage assay and an oligonucleotide DNA cleavage assay.

In some embodiments, the nuclease, preferably Cas9, can be provided as a protein, RNA, DNA, or an expression vector comprising a nucleic acid that encodes the nuclease. In some further embodiments, the guide RNA can be provided as an RNA molecule (gRNA), DNA molecule, or as an expression vector comprising a nucleic acid that encodes the gRNA. In some even further embodiments, the gRNA may be provided as one or more, e.g. as two, three, four, five, six, seven, eight, nine, or ten, RNA molecules (gRNA), DNA molecules, or expression vectors comprising a nucleic acid that encodes the gRNA, or any combination thereof.

Cas9-encoding and/or gRNA-encoding DNA can be administered to subjects or delivered into cells by methods well known in the art. For example, they can be delivered, e.g., by one or more vectors (e.g., viral or non-viral vectors/viruses or plasmids), non-vector based methods (e.g., using naked DNA or DNA complexes), or a combination thereof.

In accordance with the above, some embodiments of the invention relate to a vector system comprising one or more vectors, preferably one or more packaged vectors, comprising:

(a) a first regulatory or control element operably linked to a sequence encoding a gRNA as disclosed herein, and

(b) a second regulatory or control element operably linked to a nucleic acid encoding a Cas protein.

Suitable regulatory or control elements are well known in the art and include enhancers and promoters, such as regulated promoters (e.g., inducible promoters), constitutive promoters, and tissue specific promoters. The promoter can be a viral promoter or a non-viral promoter.

In some embodiments, a vector can also comprise a sequence encoding a signal peptide for targeted localization, fused to a sequence encoding the Cas9 molecule and/or the gRNA molecule. For example, a vector can comprise a nuclear localization sequence (e.g., from SV40) fused to the Cas9-encoding and/or the gRNA-encoding nucleic acid sequence.

Suitable viral vectors/viruses for use in the present invention include, but are not limited to, retroviruses, lentiviruses, adenovirus, adeno-associated virus (AAV), vaccinia viruses, poxviruses, and herpes simplex viruses.

Usually, viral vectors used in gene therapy are generated by a producer cell line that packages a nucleic acid vector into a viral particle. In some embodiments, the packaging cell line contains a helper plasmid encoding necessary viral genes. Those skilled in the art can easily select a suitable packaging cell line depending on the type of the viral vector to be used. Packaging cell lines as well as viral vectors are readily available in the art.

As set forth above, Cas9- and/or gRNA-encoding DNA may in some embodiments be delivered by a non-vector based method (e.g., using naked DNA or DNA complexes). For example, the DNA can be delivered by electroporation, gene gun, sonoporation, magnetofection, calcium phosphates, lipid-mediated transfection, or a combination thereof.

In some embodiments, the delivery vehicle may be a biological non-viral delivery vehicle such as an attenuated bacterium, a genetically modified bacteriophage, or a mammalian virus-like particle as is well known in the art.

In some other embodiments, the non-viral delivery vehicle may be a dendrimer or a nanoparticle. The nanoparticle may be an inorganic nanoparticle such as a magnetic nanoparticle (e.g., Fe₃MnO₂), or silica. The outer surface of the nanoparticle may be conjugated with a positively charged polymer (e.g., polyethylenimine, polylysine, polyserine) which allows for attachment (e.g., conjugation or entrapment) of payload. In some embodiments, the non-viral vector is an organic nanoparticle, e.g. a one that entraps the payload inside the nanoparticle. Exemplary organic nanoparticles include SNALP liposomes that contain cationic lipids together with neutral helper lipids which are coated with polyethylene glycol (PEG) and protamine and nucleic acid complex coated with lipid coating.

In some embodiments, the vehicle may have targeting modifications to increase target cell update of nanoparticles and liposomes, including but not limited to cell specific antigens, monoclonal antibodies, single chain antibodies, aptamers, polymers, sugars, and cell penetrating peptides. In some embodiments, the vehicle may use fusogenic and endosome-destabilizing peptides/polymers; while in some other embodiments, the vehicle may undergo acid-triggered conformational changes (e.g., to accelerate endosomal escape of the cargo). In some embodiments, a stimuli-cleavable polymer may be used, e.g., for release in a cellular compartment. For example, disulfide-based cationic polymers that are cleaved in the reducing cellular environment may be used.

In a preferred embodiment, the delivery vehicle may be a nanoparticle coated with an anti-cancer cell antibody for targeted delivery of the cargo into cancer cells.

In some embodiments, the SHANK3 inhibiting agent is a binding molecule capable of specifically binding to SHANK3 such that the function of SHANK3 is inhibited. In some embodiments, the interaction of SHANK3 with RAS is prevented, disrupted, impeded or reduced leading to release of active RAS and, consequently, activation of the RAS pathway. Said agent may be, without limitation, an antibody or a fragment or variant thereof, a nanobody, an affibody, an aptamer, a peptide, such as a blocking peptide, or a small molecule.

In accordance with the above, the SHANK3 inhibiting agent can in some embodiments be a peptide that disrupts the RAS-SHANK3 interaction via binding to SHANK3 at the RAS-binding interface. Preferably, this interface comprises an area encompassed by or located in the vicinity of the RAS-binding residues corresponding to the R12, K22 and R25 residues in human SHANK3 polypeptide having an amino acid sequence set forth in SEQ ID NO: 1, or to the R87, K97 and R100 residues in human SHANK3 polypeptide having an amino acid sequence set forth in SEQ ID NO: 36.

In some embodiments, the RAS-binding interface of SHANK3 is formed by an SPN domain comprising at least amino acids corresponding to R12, K22, R25 and R38, preferably at least amino acids corresponding to R12, K22 and R25, more preferably at least amino acids corresponding to R12 and K22, in the human SHANK3 (SEQ ID NO. 1) or in the human SPN domain of SHANK3 (SEQ ID NO. 11). Residues R12, K22, R25 and R38 of SEQ ID NO: 1 and 11 correspond to residues R87, K97, R100 and R113 in SEQ ID NO: 36. In some embodiments, the RAS-binding interface of SHANK3 comprises or consists of an amino acid sequence depicted in SEQ ID NO. 11. These alternative ways of defining the RAS-binding interface apply to all embodiments relating to the RAS-binding interface although not repeated each and every time said interface is discussed.

In some further embodiments, the SHANK3 inhibiting agent can be a peptide that disrupts the RAS-SHANK3 interaction via binding to SHANK3 and allosterically altering the RAS-binding interface of SHANK3 rendering it incapable of RAS binding. Preferably, this interface comprises an area encompassed by or located in the vicinity of the RAS-binding residues corresponding to the R12, K22, R25 and R38 residues in human SHANK3 of SEQ ID NO: 1, corresponding to the R97, K97, R100 and R113 residues in human SHANK3 of SEQ ID NO: 36.

In some still further embodiments, the inhibiting agent can be a peptide that binds SHANK3 and is be linked to a protein degradation system resulting in loss of the SHANK3 protein.

In some embodiments, the SHANK3 inhibiting agent is an anti-SHANK3 antibody or another binder-molecule, e.g. a nanobody, an affibody or an aptamer. As used herein, the term “antibody” refers to an immunoglobulin structure comprising two heavy (H) chains and two light (L) chains inter-connected by disulphide bonds. Antibodies can exist as intact immunoglobulins or as any of a number of well-characterized antigen-binding fragments or single chain variants thereof, all of which are herein encompassed by the term “antibody”. Non-limiting examples of said antigen-binding fragments include Fab fragments, Fab′ fragments, F(ab′)2 fragments, Fv fragments, scFv fragments (i.e. single-chain variable fragments), nanobodies (i.e. monomeric variable domains of camelid heavy chain antibodies) and these fragments engineered to form fusions with FC region. Said fragments and variants may be produced by recombinant DNA techniques, or by enzymatic or chemical separation of immunoglobulins as is well known in the art. The term “antibody” also includes, but is not limited to, polyclonal, monoclonal, and recombinant antibodies of isotype classes IgA, IgD, IgE, IgG, and IgM and sub-types thereof. Means and methods for producing antibodies are readily available in the art.

The term “binding molecule” includes protein engineered molecules that bind to SHANK that are based on non-antibody protein scaffold formats such as (but not limited to) affibodies or oligonucleotide based binders such as aptamers. In this context, both single-chain antibody fragments and nanobodies can be expressed in cells from plasmids/virus vectors and they can efficiently bind to their targets and interfere with function in cells.

More specifically, the SHANK3 inhibiting agent can be an antibody or other binding structure such as an antibody fragment, affibody, nanobody or aptamer that binds to SHANK3 on the RAS-interacting interface and disrupts the RASSHANK3 interaction. Preferably, the interface comprises an area encompassed by or located in the vicinity of the RAS-binding residues corresponding to the R12, K22, R25 and R38 residues in human SHANK3 of SEQ ID NO:1, corresponding to the R97, K97, R100 and R113 residues in human SHANK3 of SEQ ID NO: 36.

The antibodies may be human or humanized antibodies. Humanized antibodies are antibodies wherein the variable region may be murine derived but which has been mutated so as to more resemble a human antibody and may contain a constant region of human origin. Fully human antibodies are antibodies wherein both the variable region and the constant region are of human origin. Means and methods for producing human and humanized antibodies are readily available in the art.

In some preferred embodiments, the SHANK3 binding molecule (e.g. antibody, affibody, nanobody or aptamer) binds specifically to the RAS-binding domain (SPN, SEQ ID NO. 11) of SHANK3 and disrupts the association between SHANK3 and RAS.

In some embodiments, the inhibiting agent may be a small-molecule inhibitor. Small-molecule inhibitors are small molecules, which can easily penetrate the cell. A small molecule is able to enter cells easily because it has a low molecular weight. Once inside the cells, it can affect other molecules, such as proteins, and may cause cells to die. This is different from drugs that have a large molecular weight, such as monoclonal antibodies, which are not able to get inside cells very easily.

Accordingly, the SHANK3 inhibiting agent can be a small molecule that disrupts the RAS-SHANK3 interaction via binding to SHANK3 at the RAS-binding interface. Preferably, this interface comprises an area encompassed by or located in the vicinity of the RAS-binding residues corresponding to the R12, K22, R25 and R38 residues in human SHANK3 of SEQ ID NO: 1, corresponding to the R97, K97, R100 and R113 residues in human SHANK3 of SEQ ID NO: 36.

In some embodiments, the SHANK3 inhibiting agent can be a small molecule that disrupts the RAS-SHANK3 interaction via binding to SHANK3 and allosterically altering the RAS-binding interface of SHANK3 rendering it incapable of RAS binding. Preferably, this interface comprises an area encompassed by or located in the vicinity of the RAS-binding residues corresponding to the R12, K22, R25 and R38 residues in human SHANK3 of SEQ ID NO:1, corresponding to the R97, K97, R100 and R113 residues in human SHANK3 of SEQ ID NO: 36.

In some further embodiments, the SHANK3 inhibiting agent can be a small molecule that binds SHANK3 and is linked to a protein degradation system resulting in loss of the SHANK3 protein.

One aspect of the present invention relates to the medicinal use of at least one SHANK3 inhibiting agent for treating RAS-dependent cancer. This aspect may be formulated e.g. as a use of at least one SHANK3 inhibiting agent for the manufacture of a medicament for use in treating, preventing or ameliorating RASdependent cancer, or as a method of treating, preventing or ameliorating RAS-dependent cancer in a subject in need thereof by administering an efficient amount of at least one SHANK3 inhibiting agent. Also provided are SHANK3 inhibiting agents for use in treating, prevention or ameliorating RAS-dependent cancer.

As used herein, the term “RAS-dependent cancer” or “RAS-driven cancer” refers to cancers that have a genetic or post-translational level alteration that results in activation of RAS. These include but are not limited to RAS amplification, RAS activating mutations, and mutations in signalling molecules that control RAS activity. Above, many examples of RAS-driven or RAS-dependent cancer have been given. These may include pancreatic cancer, lung cancer, colorectal cancer, ovarian cancer, melanoma, urinary bladder carcinoma, thyroid carcinoma, hematopoietic malignancy, liver carcinoma, breast cancer, neuroblastoma, cervix adenocarcinoma, head and neck carcinoma, stomach cancer, biliary tract adenocarcinoma, angiosarcoma, malignant fibrous histiocytoma, or any other cancer that is RAS-dependent, RAS-driven or has a mutation upstream of RAS pathway. In some embodiments, the RAS-dependent cancer is RAS-dependent pancreatic cancer or RAS-dependent lung cancer.

A person with ordinary skill in the art can easily determine whether a given cancer is RAS-dependent or not using means and methods readily available in the art. For example, RNAi-based assays can be used to quantify RAS dependency and identify cancer cells that do or do not require KRAS to maintain viability.

As used herein the term “RAS amplification” refers to the amplification of the RAS gene itself.

As used herein, the term “RAS activating mutation” refers to a situation where the copy number of RAS gene is normal, but due to an activating mutation cells express RAS which is constitutively active. “Mutations in signalling molecules that control RAS activity” refers to a situation where an upstream signalling molecule that activates RAS is constitutively active or a RAS inhibiting molecule (such as but not limited to RAS GTPase activating proteins) is lost or rendered non-functional.

In some embodiments, the RAS-driven or RAS-dependent cancer is characterized by one or more RAS mutations, i.e. it is a cancer that does not harbour wild-type RAS. However, cancers with wild-type RAS may also be RAS-driven or RAS-dependent, for instance, owing to mutations in signalling molecules that control RAS activity.

In some embodiments, the RAS-driven or RAS-dependent cancer is characterized by one or more oncogenic KRAS mutations. In other words, in such cases said RAS-driven or RAS-dependent cancer is not a KRAS wild-type cancer. Non-limiting examples of KRAS mutations leading to RAS activation include mutations in the KRAS gene at codons encoding amino acid residues at positions 5, 8, 9, 12, 13, 14, 19, 21, 22, 33, 36, 58, 59, 61, 62, 63, 68, 71, 72, 92, 117, 119, and 146 of SEQ ID NO: 8 or 9.

In some embodiments, preferred activating KRAS mutations include, but are not limited to, the following:

mutation of the glycine residue at the amino acid position 12 of SEQ ID NO: 8 or 9 to an amino acid residue other than glycine, preferably to alanine (G12A), cysteine (G12C), aspartic acid (G12D), arginine (G12R), serine (G12S) or valine (G12V);

mutation of the glycine at the amino acid position 13 of SEQ ID NO: 8 or 9 to an amino acid residue other than glycine, preferably to cysteine (G13C) or aspartic acid (G13D);

mutation of the leucine at the amino acid position 19 of SEQ ID NO: 8 or 9 to an amino acid residue other than leucine, preferably to phenylalanine (L19F);

mutation of the glutamine at the amino acid position 22 of SEQ ID NO: 8 or 9 to an amino acid residue other than glutamine, preferably to lysine (Q22K);

mutation of the aspartic acid at the amino acid position 33 of SEQ ID NO: 8 or 9 to an amino acid residue other than aspartic acid, preferably to glutamic acid (D33E);

mutation of the alanine at the amino acid position 59 of SEQ ID NO: 8 or 9 to an amino acid residue other than alanine, preferably to glycine (A59G);

mutation of the glutamine at the amino acid position 61 of SEQ ID NO: 8 or 9 to an amino acid residue other than glutamine, preferably to histidine (Q61H), leucine (Q61L), or arginine (Q61R);

mutation of the glutamic acid at the amino acid position 62 of SEQ ID NO: 8 or 9 to an amino acid residue other than glutamic acid, preferably to lysine (E62K);

mutation of the lysine at the amino acid position 117 of SEQ ID NO: 8 or 9 to an amino acid residue other than lysine, preferably to asparagine (K117N); and

mutation of the alanine at the amino acid position 146 of SEQ ID NO: 8 or 9 to an amino acid residue other than alanine, preferably to threonine (A146T).

Further non-limiting examples of preferred activating mutations include KSE, G13V, V14I, T58K, A59E, A59T, Q61E, Q61K, E63K, Y71C, A146V, A146P, I36M, R68M, R68S, and D92Y. Still further activating KRAS mutations can be found in The Cancer Genome Atlas (TCGA) database as well as in scientific literature, all of which mutations are incorporated herein by reference.

As used herein, the term “subject” refers to an animal, preferably to a mammal, more preferably to a human. Herein, the terms “human subject”, “patient” and “individual” are interchangeable.

As used herein, the term “treatment” or “treating” refers not only to complete cure of a disease, but also to alleviation, and amelioration of a disease or symptoms related thereto.

As used herein, the term “preventing” refers to any action resulting in suppression or delay of the onset of the disease.

Moreover, the invention relates to a use of SHANK3 for screening and/or identifying potential therapeutic agents for treating a RAS-dependent cancer. This aspect of the invention is not limited to any particular technique for identifying said therapeutic agents. Non-limiting examples of suitable techniques include, but are not limited to, in vitro screening assays such as binding assays and cell-based assays, as well as in silico screening assays.

In some embodiments, SHANK3 and a RAS isoform are provided in isolated form. Preferably, said isolated SHANK3 and the RAS isoform, or a biological sample comprising the same, are brought into contact with a test agent. If said test agent is capable of diminishing or abolishing the interactions of SHANK3 and RAS, it may be regarded as a potential inhibitor of SHANK3 function. The SHANK3 inhibiting activity of the test agent may be verified by any appropriate biochemical assay and/or cell-based assay. Non-limiting examples of suitable cell-based assays include in-cell western assays, such as those wherein increased ERK activity in RAS-dependent cancer cells indicates or verifies that indeed the test agent is an inhibitor of SHANK3 action. Further non-limiting examples of cell-based assays include those wherein the effect of the test agent on cell number, preferably on the number of RAS-dependent cancer cells, is used as the readout. In such assays, reduced or diminished number of RAS-dependent cancer cells indicates or verifies that indeed the test agent is an inhibitor of SHANK3 function.

Accordingly, in some embodiments, the invention provides a method for identifying a compound to treat a RAS dependent cancer, the method comprising:

i. contacting a SHANK3 and a RAS polypeptide with a test compound,

ii. determining if the compound reduces binding between SHANK3 and RAS,

iii. selecting compounds that inhibit binding. Preferably, said binding is inhibited by at least 10%, more preferably by at least 20%, even more preferably by at least 30%, by at least 40%, by at least 50%, by at least 60%, by at least 70%, by at least 80%, by at least 90% or even by 100% as compared to binding in the absence of the test compound.

Also provided is a method for identifying a candidate compound for treatment of RAS dependent cancer, the method comprising:

i. contacting a SHANK3 polypeptide and a RAS polypeptide with a test compound,

ii. determining whether the test compound reduces binding between SHANK3 and RAS, and

iii. identifying the test compound as a candidate compound for treatment of RAS dependent cancers, if said binding is reduced. Preferably, said binding is reduced by at least 10%, more preferably by at least 20%, even more preferably by at least 30%, by at least 40%, by at least 50%, by at least 60%, by at least 70%, by at least 80%, by at least 90% or even by 100% as compared to binding in the absence of the test compound.

In the above methods, SHANK3, RAS, or both may be labelled with a detectable label using means and methods well known in the art. Alternatively or in addition, SHANK3 or RAS may be immobilized on a surface using means and methods well known in the art.

Preferably, the SHANK3 polypeptide contacted with the test compound is the SPN-domain of SHANK3 (SEQ ID NOs.: 11, 12 or 13). Preferably, the test compound is selected in silico or through other methods including but not limited to screening of compound libraries. Preferably, the inhibition is verified in a cellular assay.

In accordance with the above, the invention also relates to a method for identifying an inhibitor for SHANK3-RAS interaction or association comprising the steps of:

(i) designing a possible inhibitory molecule based on SHANK3-SPN domain sequence (SEQ ID NO.:11, 12 or 13):

(ii) determining whether the molecule binds to SHANK3-SPN

(iii) determining whether the molecule diminishes SHANK3-RAS interaction, and

(iv) determining whether the molecule increases ERK activity in cancer cells and/or determining whether the molecule diminishes the number of RAS-dependent cancer cells in vitro; wherein

if the molecule increases the ERK-activity in the RAS-dependent cancer cells and/or if the molecule diminishes the number of RAS-dependent cancer cells, the molecule has the capacity for inhibiting SHANK3-RAS interaction or association. Preferably, an in-cell western assay is used for determining whether the inhibitor increases ERK activity in cancer cells.

Preferably, but without limitation, the ability of the compound to reduce the binding between SHANK3 and RAS, is determined for example by one of the following assays:

i. SHANK3-SPN fragment is a recombinant, purified, his-tagged protein. RAS is recombinant, purified and loaded with a non-hydrolysable GTP analog such as GTPgammaS or GppNHp. Their interaction in the presence or absence of an inhibitor is measured using microscale thermophoresis (MST).

ii. SHANK3-SPN fragment is a recombinant, purified, his-tagged protein coupled to Ni-NTA beads. RAS is recombinant, purified, labelled covalently with a fluorescent dye and loaded with a non-hydrolysable GTP analog such as GTPgammaS or GppNHp. Their interaction in the presence or absence of an inhibitor is measure by analyzing bead fluorescence using flow cytometry.

iii. RAS is recombinant, purified and loaded with a non-hydrolysable GTP analog such as GTPgammaS or GppNHp. It is immobilized to the bottom of a microtiter well. SHANK3-SPN fragment is a recombinant, purified, GST-tagged protein. Their interaction in the presence or absence of an inhibitor is detected by washing the wells after incubation and detecting bound GST-tagged protein with anti-GST antibodies coupled either to HRP to allow for colorimetric ELISA assay detection or conjugated with Europium-chelates to allow for timeresolved fluorometry-based detection.

iv. RAS is recombinant, purified, labelled covalently with a fluorescent dye and loaded with a non-hydrolysable GTP analog such as GTPgammaS or GppNHp. SHANK3-SPN fragment is a recombinant, purified, GST-tagged protein. It is immobilized to the bottom of a microtiter well. Their interaction in the presence or absence of an inhibitor is detected by washing the wells after incubation and measuring fluorescence with a plate-reader instrument.

In addition, the invention relates to a kit comprising an isolated SHANK3 polypeptide, or a fragment thereof, preferably the SPN-domain of SHANK3 (SEQ ID Nos.: 11, 12 and 13), and an isolated RAS-isoform polypeptide, or a fragment thereof. In some embodiments, the SHANK3 polypeptide, preferably the SHANK3-SPN fragment, is a recombinant, purified, his-tagged protein. In some embodiments, the RAS polypeptide is recombinant, purified and loaded with a nonhydrolysable GTP analog such as GTP-gammaS or GppNHp. The kit may be used for screening or identifying one or more agents for treating or preventing a RAS-dependent cancer.

In some embodiments of the kit and of the above-described screening and/or identification methods, the isolated SHANK3 polypeptide has at least 80% identity, preferably at least 85% identity, more preferably at least 90% identity, more preferably 95% identity, more preferably 98% identity, more preferably 99% identity to SEQ ID NO: 1, 2, 3, 11, 12, 13 or 36, or comprises or consists of SEQ ID NO: 1, 2, 3, 11, 12, 13 or 36. In some further embodiments, the isolated SHANK3 polypeptide is a conservative sequence variant of a SHANK3 polypeptide comprising or consisting of SEQ ID NO: 1, 2, 3, 11, 12,13 or 36. Furthermore, the isolated SHANK3 polypeptide may be any SHANK3 variant, such as a splice variant, provided that it comprises an SPN domain (SEQ ID NO: 11, 12 and 13) that has retained its function, i.e. is able to interact with a RAS isoform. Preferably, the RAS-binding residues corresponding to the R12, K22, R25 and R38 residues, more preferably at least residues R12 and K22, in the human SHANK3 (SEQ ID NO. 1) or in the human SPN domain (SEQ ID NO. 11) are intact. Accordingly, the RAS-binding residues corresponding to the R97, K97, R100 and R113 residues, more preferably at least residues R97 and K07, in the human SHANK3 of SEQ ID NO. 36 are preferably intact.

In some embodiments of the kit and of the above-described screening and/or identification methods, the isolated RAS-isoform has at least 80% identity, preferably at least 85% identity, more preferably at least 90% identity, more preferably 95% identity, more preferably 98% identity, more preferably 99% identity to any one of SEQ ID NO: 6, 7, 8, 9, 10, 15 or 16, or comprises or consists of SEQ ID NO.: 6, 7, 8, 9, 10, 15 or 16. In some further embodiments, the isolated RAS polypeptide is a conservative sequence variant of a RAS polypeptide comprising or consisting of SEQ ID NO: 6, 7, 8, 9, 10, 15 or 16, or fragment thereof. Preferably, the variant or the fragment comprises or consists a RAS domain responsible for the interaction with SHANK3, Basically, the RAS polypeptide may any RAS variant provided that its ability to interact with SHANK3 is retained.

In some embodiments, the RAS polypeptide is a KRAS polypeptide comprising one or more oncogenic mutations. Thus, in any of the embodiments described above, said polypeptide of SEQ ID NO: 8 or 9 may comprise, for example, one or more mutations at amino acid positions 5, 8, 9, 12, 13, 14, 19, 21, 22, 33, 36, 58, 59, 61, 62, 63, 68, 71, 72, 92, 117, 119, and/or 146. Non-limiting examples of preferred mutations at these positions include those set forth above, for example, G12A, G12C, G12D, G12R, G125, G12V, G13C, G13D, L19F, Q22K, D33E, A59G, Q61H, Q61L, Q61R, E62K, K117N, A146T, K5E, G13V, V14I, T58K, A59E, A59T, Q61E, Q61K, E63K, Y71C, A146V,A146P, I36M, R68M, R68S, and D92Y. In some embodiments, the KRAS polypeptide comprises or consists of SEQ ID NO: 15 or 16, or a conservative sequence variant or a fragment thereof, provided that its ability to interact with SHANK3 is retained.

The term “conservative sequence variant”, as used herein, refers to amino acid sequence modifications, which do not significantly alter the biological properties of the polypeptide in question. Conservative polypeptide variants include variants arising from one or more amino acid substitutions with similar amino acids well known in the art (e.g. amino acids of similar size and with similar charge properties).

In some embodiments, the isolated SHANK3 may be a recombinant SHANK3 polypeptide and/or the isolated RAS may be a recombinant RAS polypeptide. Said polypeptide(s) may comprise small peptide or fusion-protein tags that facilitate, for example, purification, isolation, immobilization and/or detection. Non-limiting examples of suitable affinity tags e.g. for purification and immobilization purposes include polyhistidine tags (His-tags), hemagglutinin tags (HA-tags), glutathione S-transferase tags (GST-tags), biotin tags, avidin tags and streptavidin tags.

In some embodiments of the kit and of the above-described screening and/or identification methods, the SHANK3, the RAS isoform or both are labelled with a detectable label,

As used herein, the term “detectable label” refers to any molecule which can be detected, either directly or indirectly. Non-limiting examples of detectable labels include optical agents such as fluorescent agents including a variety of organic and/or inorganic small molecules and a variety of fluorescent proteins and derivatives thereof, phosphorescent agents, luminescent agents such as chemiluminescent agents, and chromogenic agents; radiolabels; and enzymes such as alkaline phosphatase (AP), or (horseradish) hydrogen peroxidase (HRP). Further suitable detectable labels are available in are available in the art. Those skilled in the art can readily select an appropriate detection technique depending on the type and species of the detectable label employed.

The spatial structure of the SPN-ARR domain of SHANK3 has been disclosed by Lilja et al. (13). In accordance with the present invention, this structure may be used for in silico screening or identification of one or more candidate compounds for the treatment of RAS-dependent cancer, as well as for de novo compound design.

One aspect of the present invention thus relates to a computer-based method of using a spatial structure of the RAS-binding interface of SHANK3 or a spatial structure of SHANK3-RAS isoform complex in a drug screening assay. Any existing library of potential ligands of SHANK3 can be examined through the use of computer modelling using a docking program such as GRAM, DOCK, AUTODOCK, MOE-DOCK or FLEXX. This procedure can include, for example, computer fitting of potential ligands to said spatial structure to determine how well the shape and the chemical structure of the potential ligand will interfere with SHANK3 interaction with RAS isoforms.

Docking algorithms can also be used to verify interactions with ligands designed de novo. As used herein, “de novo compound design” refers to the process wherein the three-dimensional structure of SHANK3 is used as a platform or basis for the rational design of compounds that will prevent or diminish the interaction of SHANK3 with RAS. Preferably said structure comprises the RAS-binging interface encompasses by or located in the vicinity of the RAS-binding residues corresponding to the R12, K22, R25 and R38 residues in the human SHANK3 (SEQ ID NO. 1). In some embodiments, the RAS-binding interface of SHANK3 is formed by an SPN domain comprising at least amino acids corresponding to R12, K22, R25 and R38, preferably at least amino acids corresponding to R12, K22 and R25, more preferably at least amino acids corresponding to R12 and K22, in the human SHANK3 (SEQ ID NO. 1) or in the human SPN domain of SHANK3 (SEQ ID NO. 11). As set forth above, residues R12, K22, R25 and R38 of SEQ ID NO: 1 and 11 correspond to residues R87, K97, R100 and R113 in SEQ ID NO: 36 representing a SHANK3 polypeptide with 75 additional N-terminal amino acids as compared to a SHANK3 polypeptide of SEQ ID NO: 1. In some embodiments, the RAS-binding interface of SHANK3 comprises or consists of an amino acid sequence depicted in SEQ ID NO. 11.

Accordingly, in some embodiments, the computer-based method for screening, identifying or designing a compound for treatment of RAS dependent cancer may be formulated as a method comprising

i. providing a spatial structure of the RAS binding domain of SHANK3 in a computer, or generating a spatial structure of the RAS binding domain of SHANK3, wherein said domain comprises at least amino acids corresponding to R12, K22, R25 and R38, preferably at least amino acids corresponding to R12, K22 and R25, more preferably at least amino acids corresponding to R12 and K22, in the human SHANK3 (SEQ ID NO. 1) or in the human SPN domain of SHANK3 (SEQ ID NO. 11), in a computer based on the spatial structure provided by Lilja et al. in Nature Cell Biology 2017 (ibid.),

ii. generating a spatial structure of potential inhibitors in a computer, and

iii. selecting potential inhibitors having a structure which can bind at least one amino acid residue of said domain. Alternatively, the RAS binding domain comprises at least amino acids corresponding to R87, K97, R100 and R113, preferably at least amino acids corresponding to R87, K97 and R100, more preferably at least amino acids corresponding to R87 and K97, in the human SHANK3 of SEQ ID NO. 36.

In some embodiments of the computer-based method, the RAS-binding domain of SHANK3 comprises or consists of an amino acid sequence depicted in SEQ ID NO. 11.

Computer programs can also be employed to estimate the attraction, repulsion, and steric hindrance between the potential ligand and SHANK3. Generally, the tighter the fit (e.g. the lower the steric hindrance, and/or the greater the attractive force) the more potent the potential drug will be since these properties are consistent with a tighter binding constant.

After selecting a potential drug by in silico computer modelling or de novo compound design, the potential drug may be produced and contacted with SHANK3 and a RAS isoform in order to detect its ability to interfere or diminish the SHANK3-RAS interaction. In a particular embodiment either the potential drug or SKANK3 or the RAS isoform is labeled using means and methods readily available in the art. In another embodiment, either SHANK3 or the RAS isoform is bound to a solid support. Non-limiting examples of high throughput techniques for assessing the binding of the potential drug to SHANK3, RAS isoform or both include microscale thermophoresis (MTS) and fluorescence-based thermal shift (FTS) assay. Non-limiting examples of high throughput techniques for determining whether or not SHANK3 interacts with a RAS isoform in the presence of the potential drug include isothermal calorimetry (ITC), surface plasmon resonance (SPR), microscale thermophoresis, fluorescence resonance energy transfer (FRET) and amplified luminescent proximity homogeneous assay screen (AlphaScreen, Perkin Elmer).

In the following, non-limiting Examples are shown.

### EXAMPLES

### Example 1. Shank3 Directly Interacts with Oncogenic KRAS

### Material and Methods

Cell Lines and Cell Culture

HEK293 (human embryonic kidney, ATCC) cells were grown in DMEM (Dulbecco's modified Eagle's medium, Sigma-Aldrich) supplemented with 10% FBS and 2 mM L-glutamine. Cells were routinely tested for mycoplasma contamination.

Transient Plasmid Transfections

Cells were seeded on a 6-well plate a day before transfection and transient plasmid transfections were performed the next day when the cell confluence was approximately 70-80%. Plasmid DNA-lipid complexes, including plasmid DNA of interest (2-4 ug), Lipofectamine 3000 Reagent (2-4 ul) and P3000™ Enhancer Reagent (5 ul) (Thermo Fisher Scientific Inc), were prepared in Opti-MEM (final volume 500 ul) according to manufacturer's instructions. Cells were cultured 1 ml of cell culture medium and plasmid DNA-lipid complexes were added dropwise on cells. Cells were incubated with transfection mix overnight, and next day the plasmid transfected cells were used for experiments.

Immunoprecipitation of RFP/GFP-Tagged Proteins

HEK293 cells were transiently transfected with (1) mRFP-tagged Shank3 WT together with GFP-tagged KRASG12V or control (GFP only) or (2) GFPtagged Shank3 SPN WT, Shank3 SPN R12E/K22D or control (GFP only) together with dsRed-tagged KRASG12V. Cells were lysed using IP buffer, cleared by centrifugation, and subjected to immunoprecipitation of RFP-tagged or GFP-tagged fusion proteins using RFP-trap or GFP-trap matrix (Chromotek, Munich, Germany), respectively. Input and precipitate samples were analyzed by Western blot.

Western Blot Analyses

Protein extracts were sonicated (0.5 min ON/0.5 min OFF totally 5 min with full power) and protein levels were measured by Bio-Rad protein quantification kit. Sample buffer was added and samples were boiled for 5 min at 95° C. heat block. Proteins were separated separated using SDS-PAGE under denaturing conditions (4-20% Mini-PROTEAN TGX Gels) and transferred into nitrocellulose membranes by semi-dry turbo blot (BioRad Laboratories). Membranes were blocked with 5% BSA in TBST (Tris-buffered saline and 0.1% Tween 20) for 1 hour at room temperature (RT). Primary antibodies were diluted in 5% BSA in TBST and incubated with membranes overnight at +4° C. After primary antibody incubation, membranes were washed three times with TBST for 5 min at room temperature. Fluorophore-conjugated or ECL HRP-linked secondary antibodies (GE Healthcare) were diluted 1:5000 in 5% BSA in TBST or in blocking buffer (ThermoScientific) in PBS (1:1) and were incubated with membranes 1 hour at RT. Membranes were scanned using an infrared imaging system (Odyssey; LI-COR Biosciences) or ECL Plus Western blotting reagent (GE Healthcare) and film was developed. Band intensity was determined using Fiji (ImageJ; National Institutes of Health; Schindelin et al., 2012) or Image Studio Lite (Licor). Following primary antibodies were used: SHANK3 (Santa Cruz, sc-30193), GFP (Abcam, ab1218) and KRAS (WH0003845M1, Sigma).

FRET Imaging Using Fluorescence Lifetime Microscopy (FLIM)

HEK293 cells were grown on coverslips overnight and transfected with an mGFP-tagged donor construct (GFP-SPNWT) and mCherry-tagged acceptor construct (mCherry-KRASG12V) using Lipofectamine® 3000 (Invitrogen) for 24 h. Cells were then fixed with 4% PFA and mounted with Mowiol 4-88 on microscope slides. Fluorescence lifetimes of the GFP-tagged donor constructs were measured using a fluorescence lifetime imaging attachment (Lambert Instruments, Leutingwolde, The Netherlands) on an inverted microscope (Zeiss Axio Observer.D1). Fluorescein (0.01 mM, pH 9) was used as a lifetime reference standard. In addition, it served to calibrate a fixed setting that allows acquisition of data from cells with comparable expression levels. Three biological repeats were performed, and the apparent fluorescence resonance energy transfer (FRET) efficiency was calculated from obtained fluorescence lifetimes.

### Results

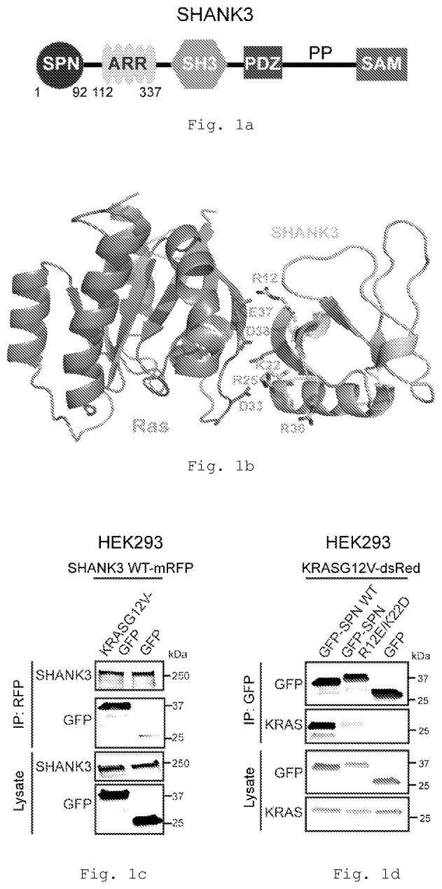

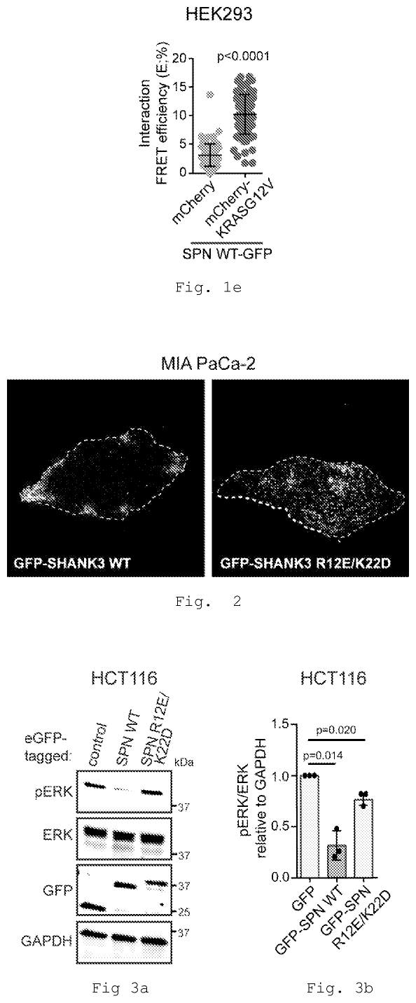

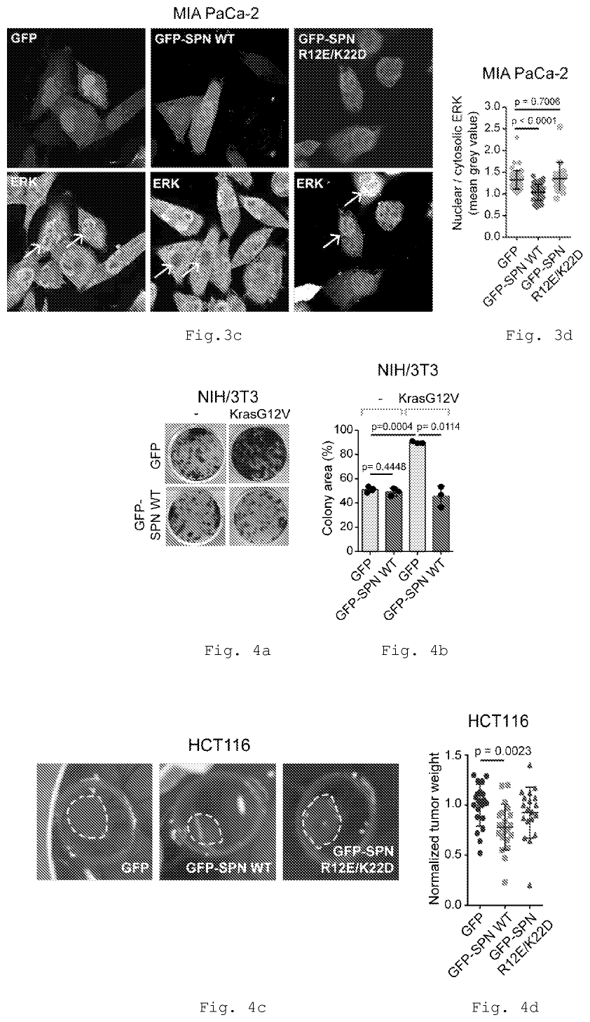

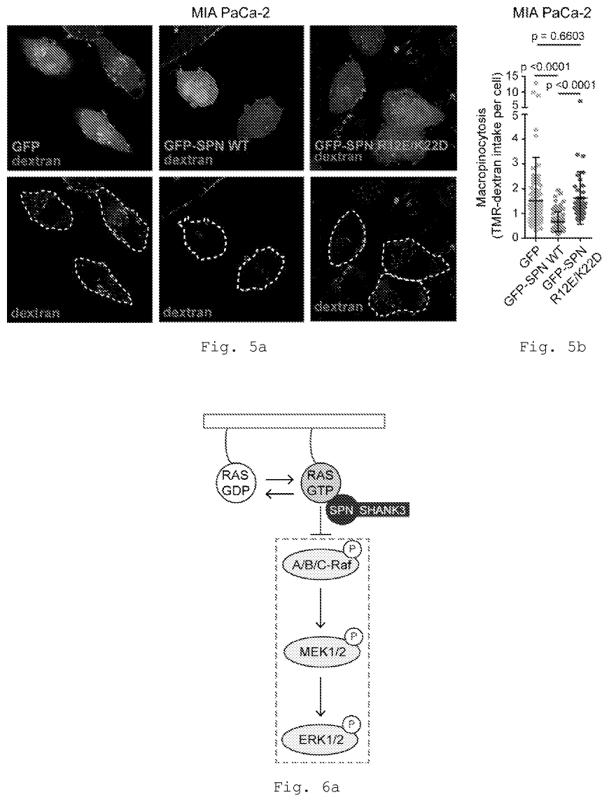

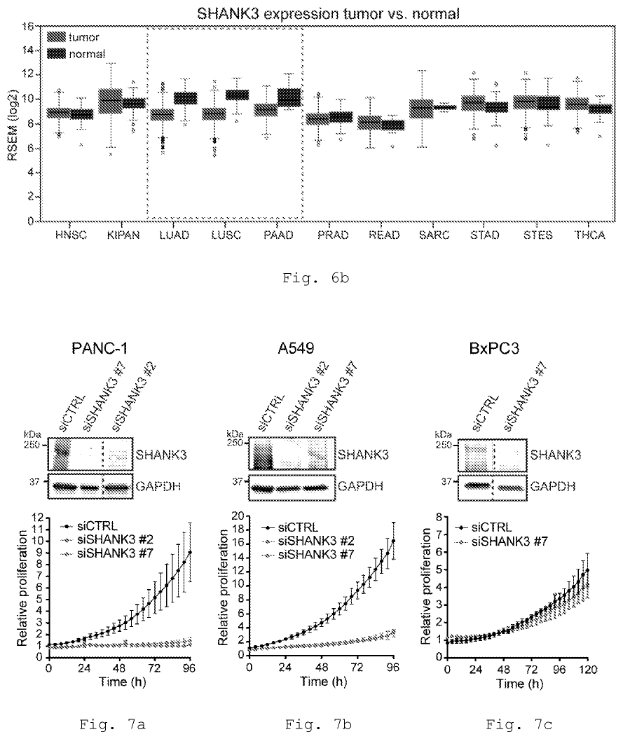

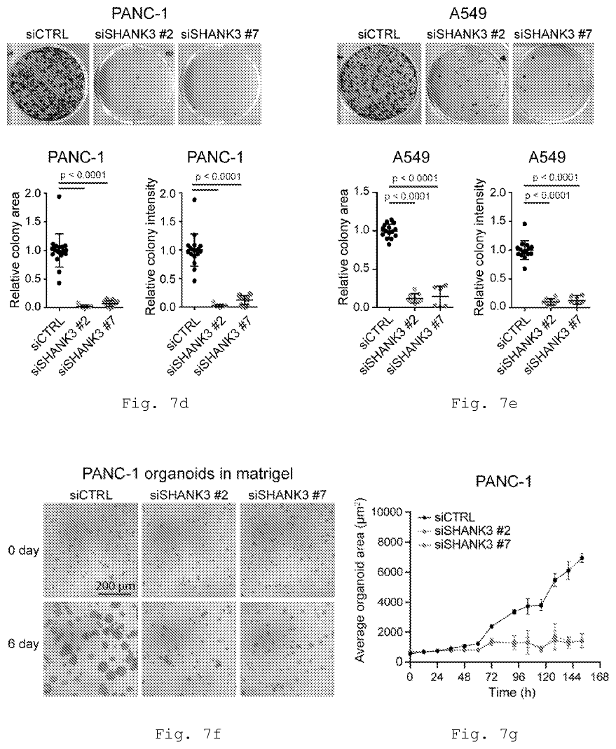

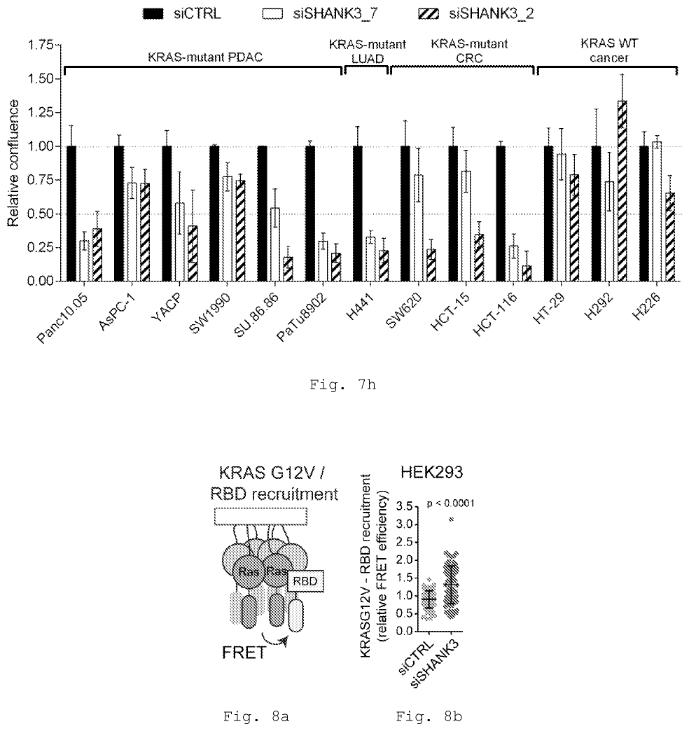

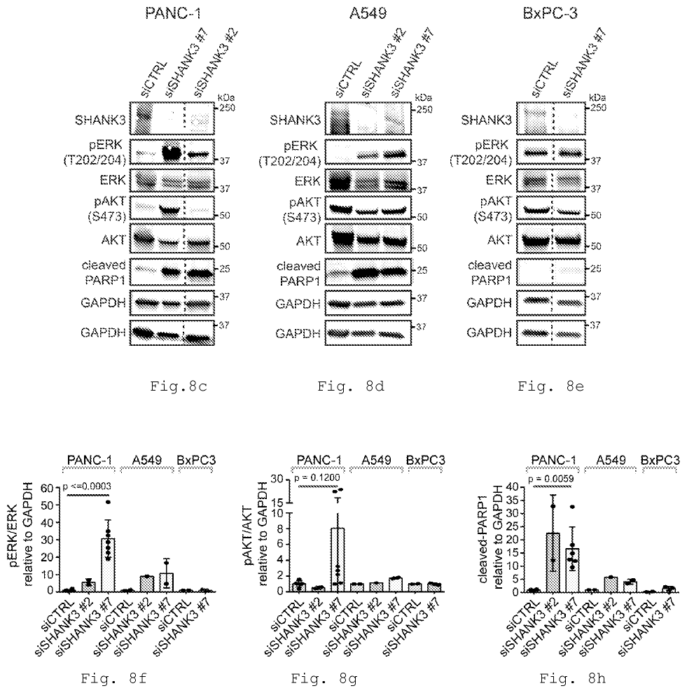

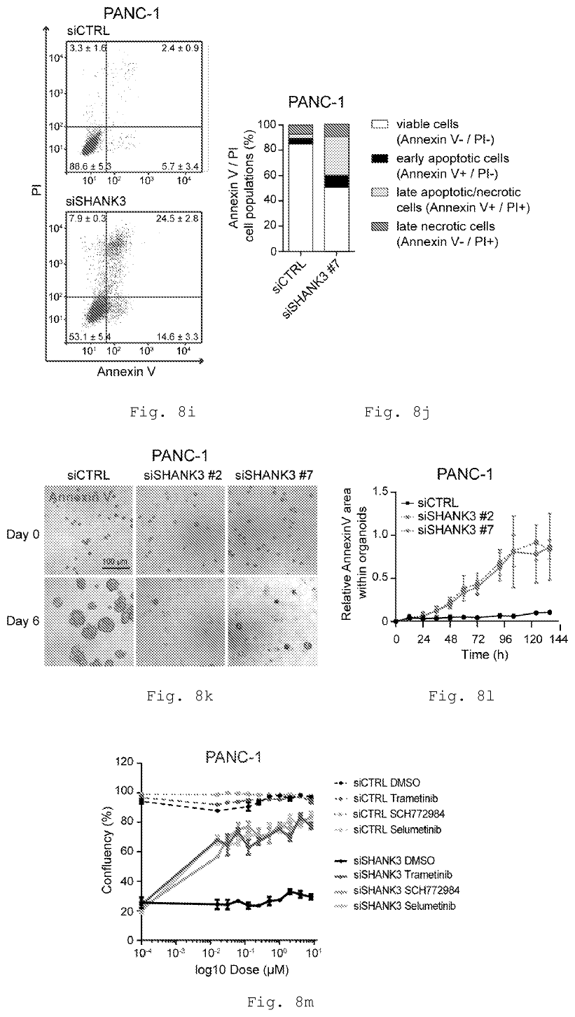

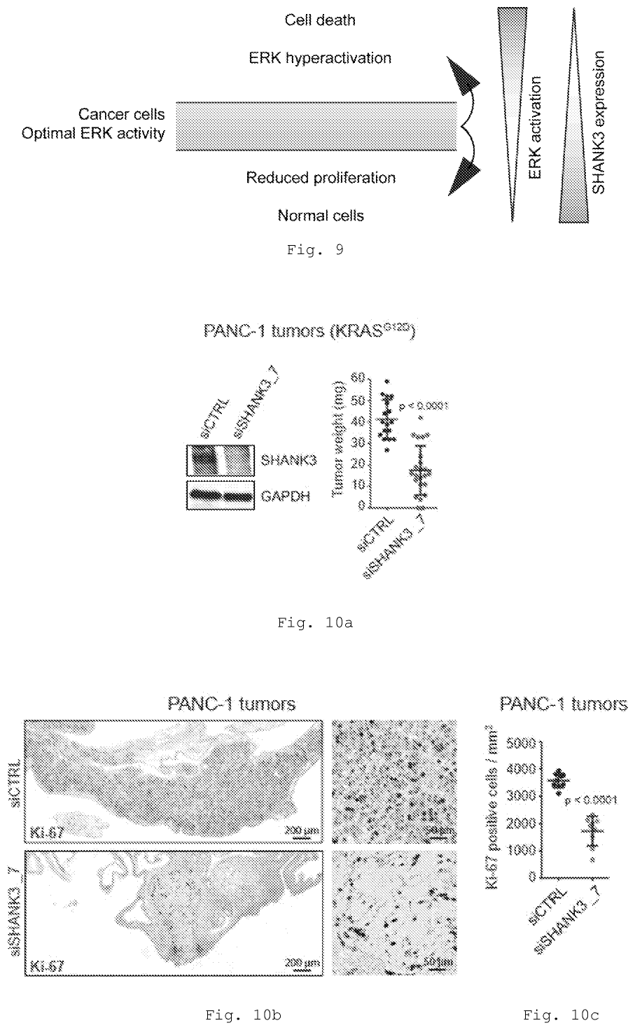

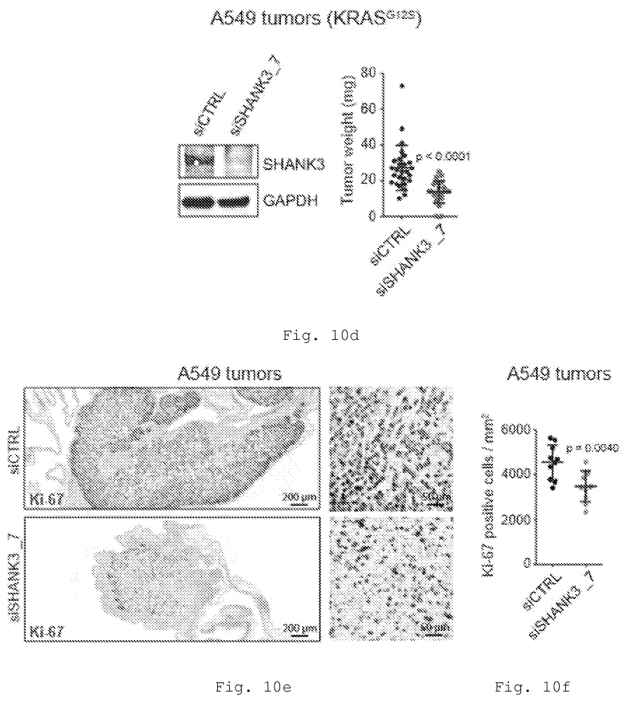

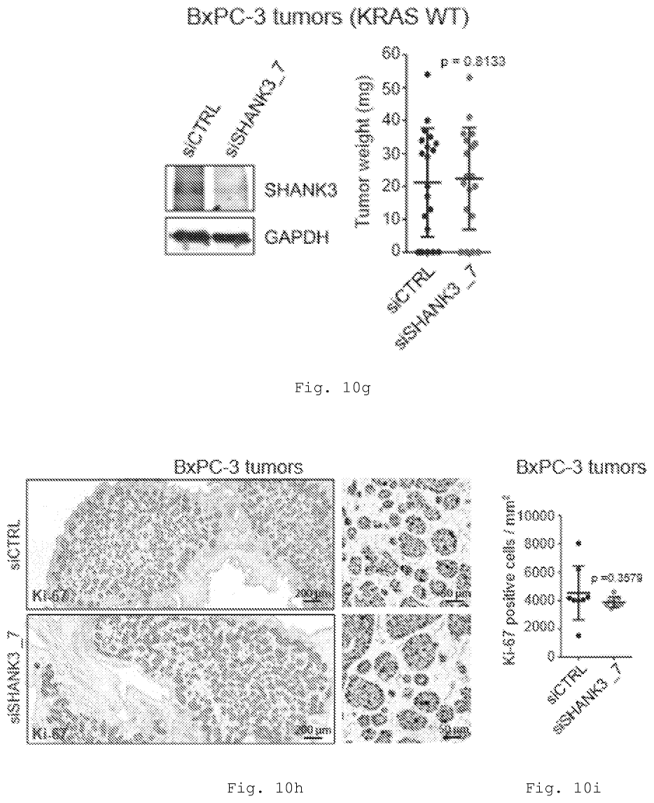

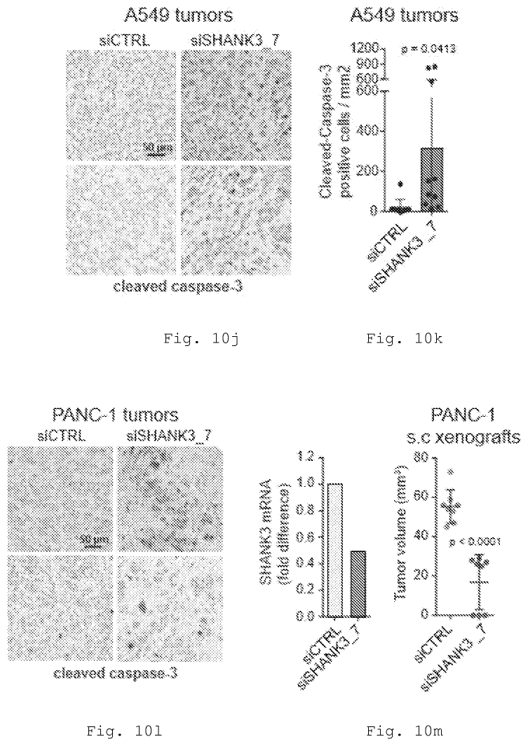

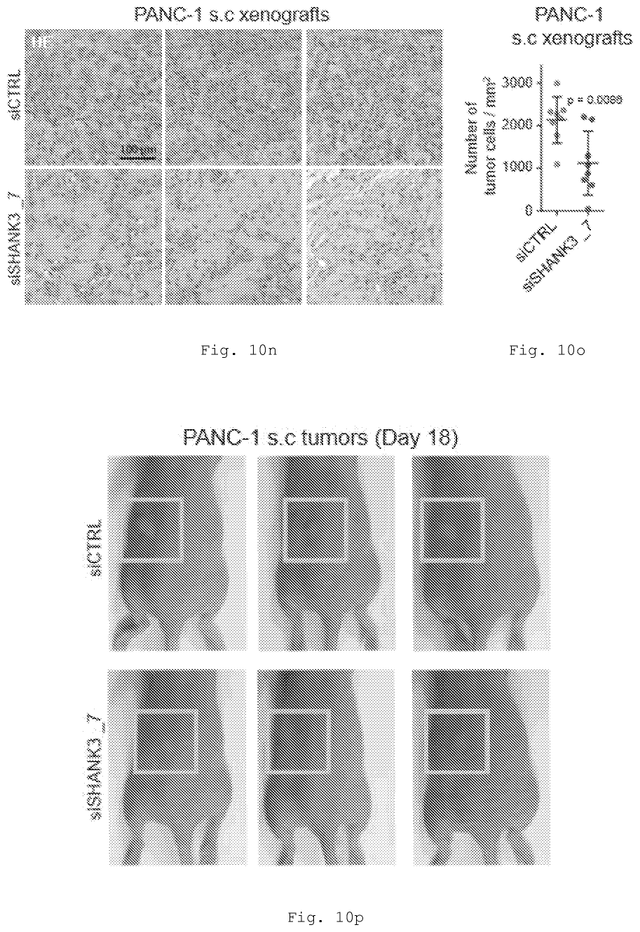

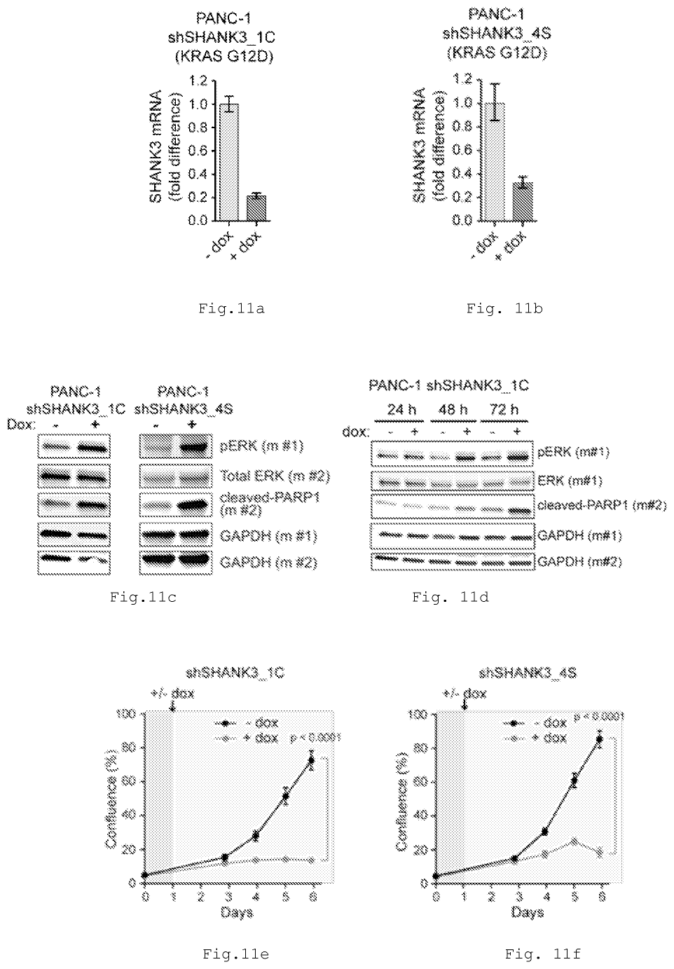

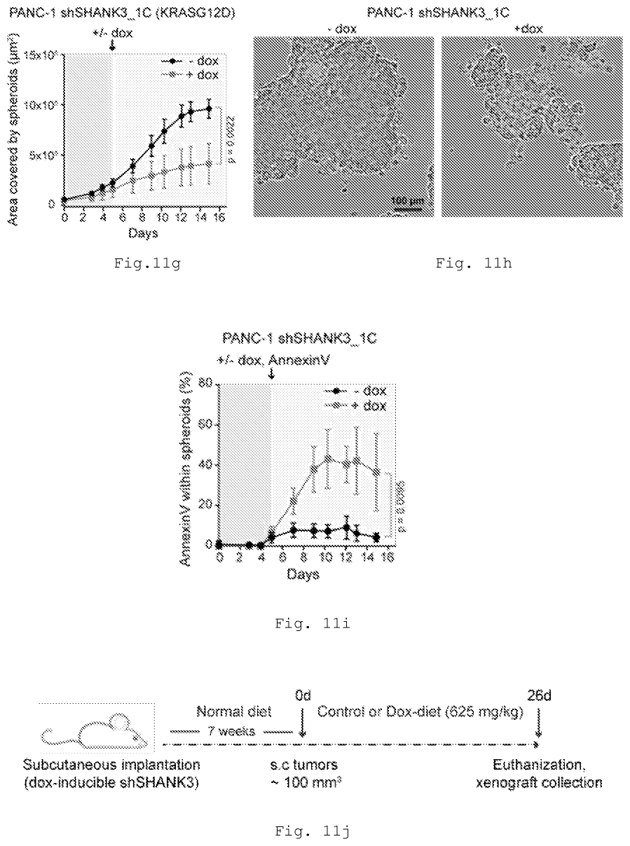

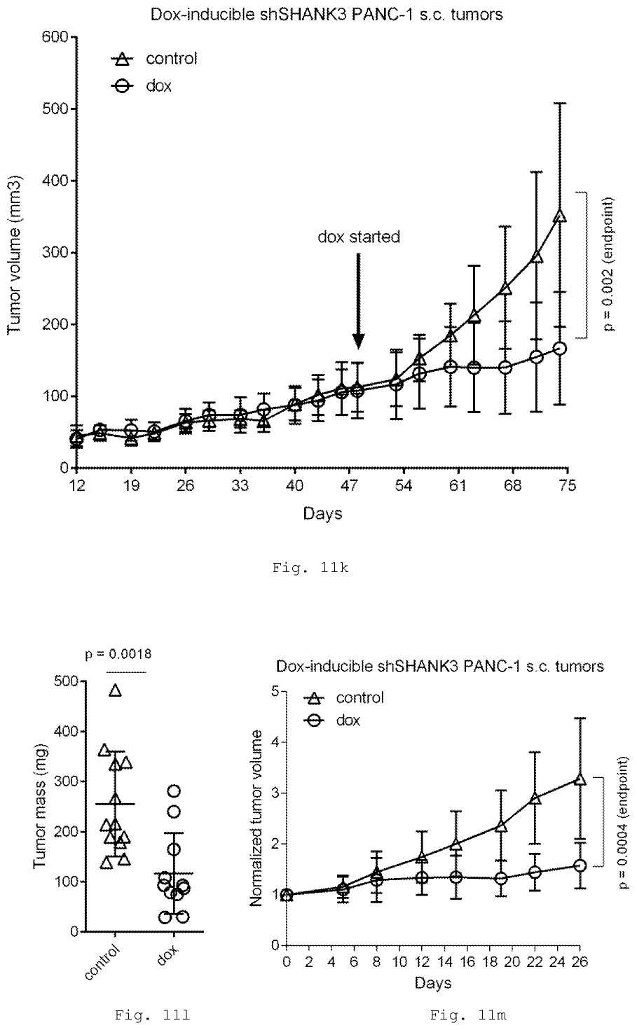

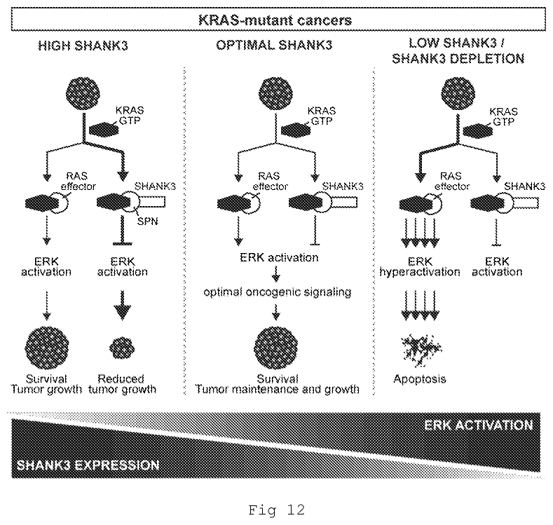

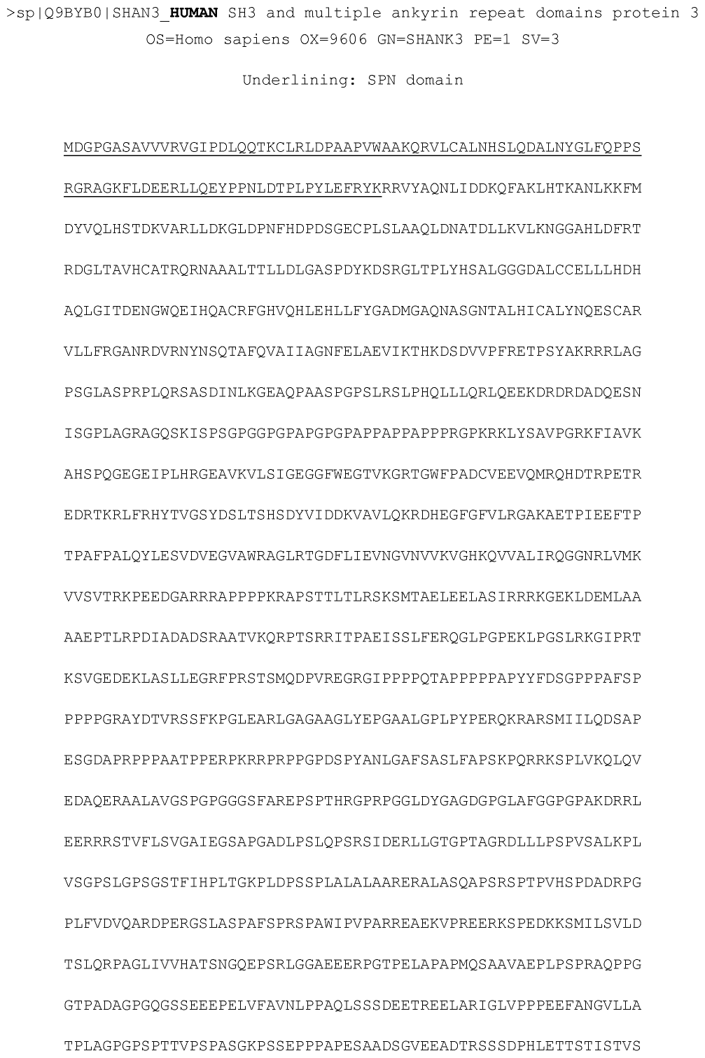

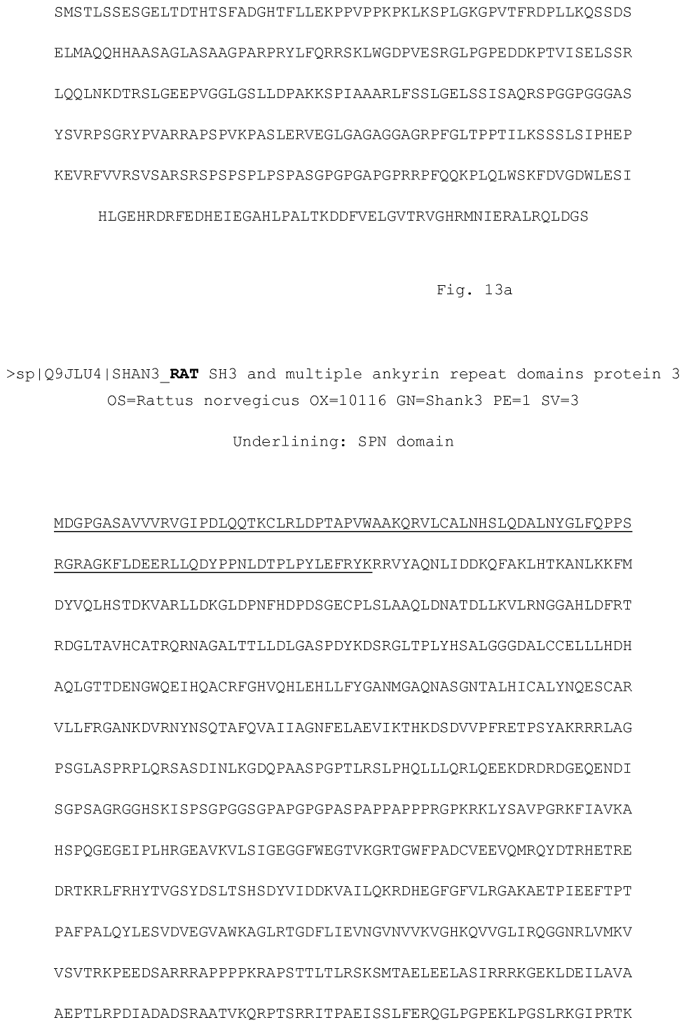

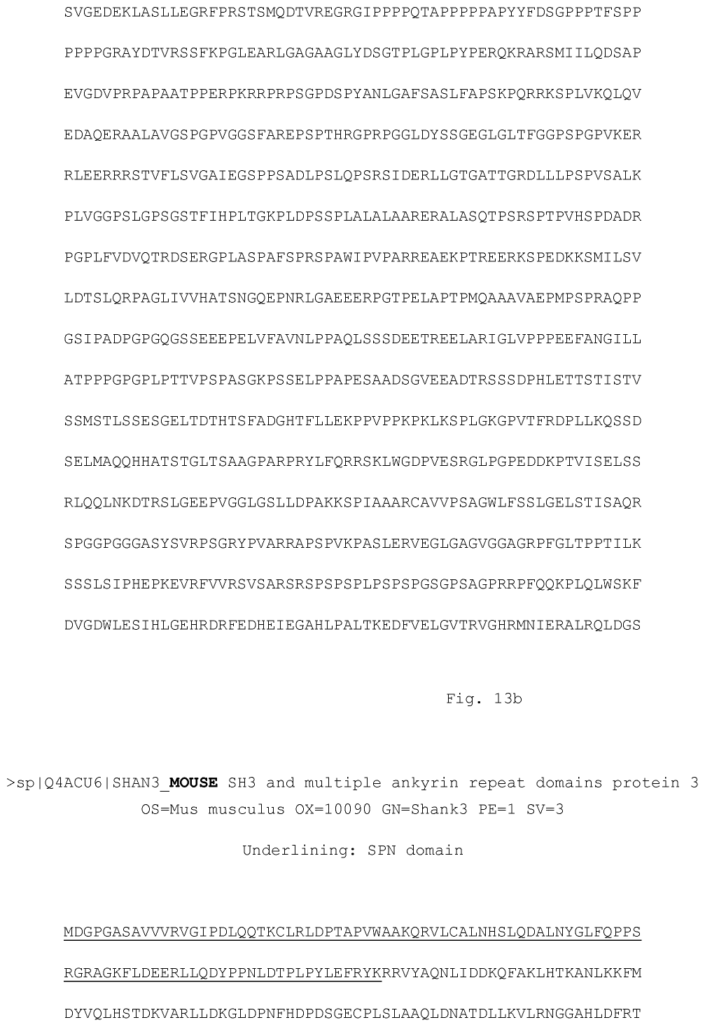

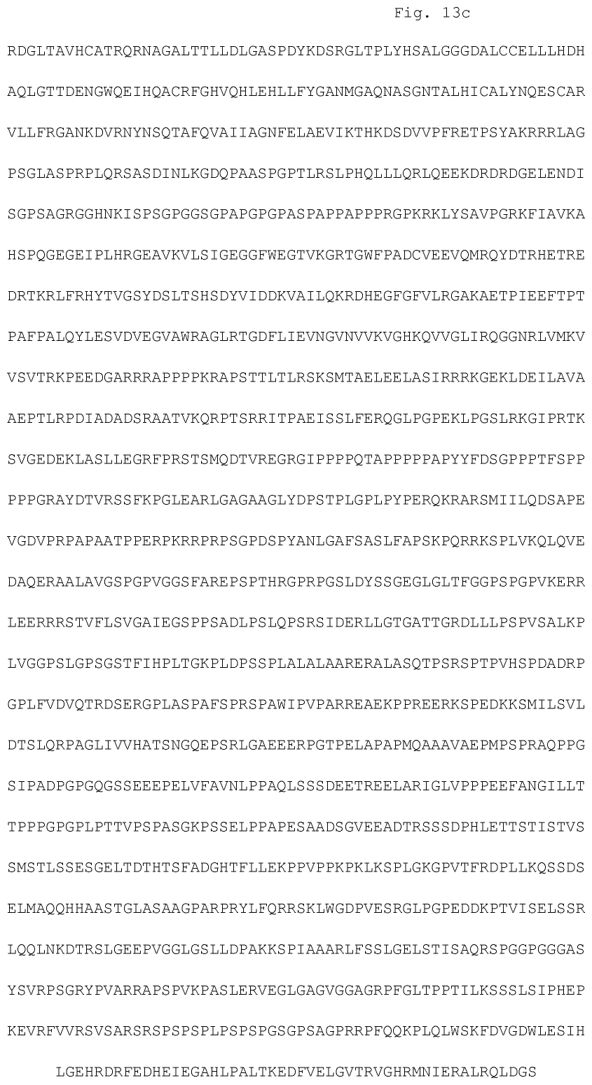

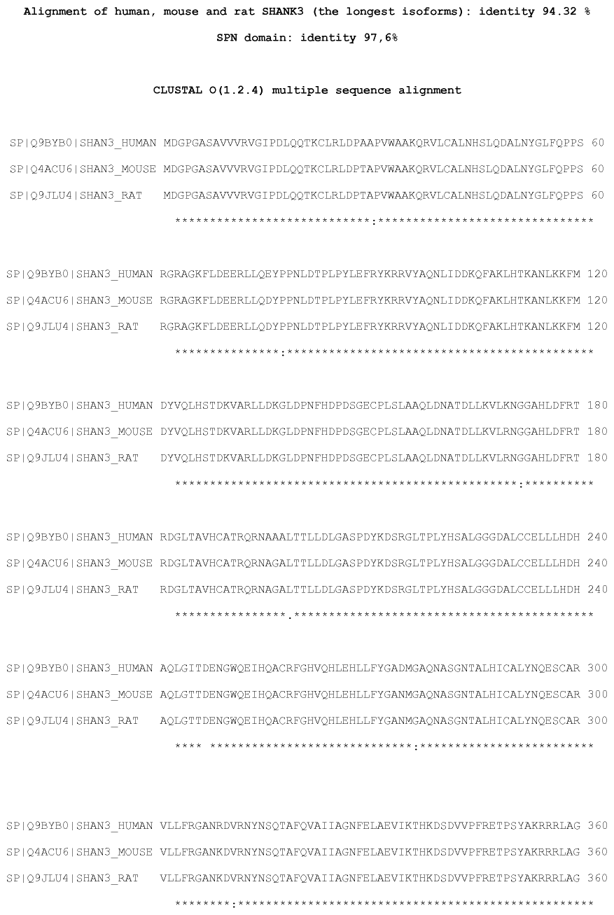

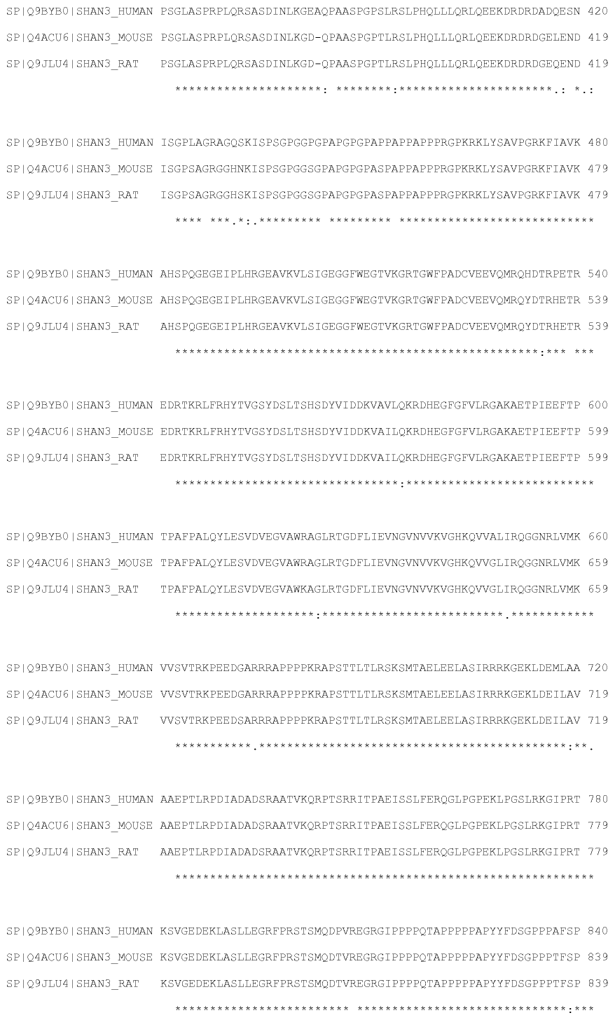

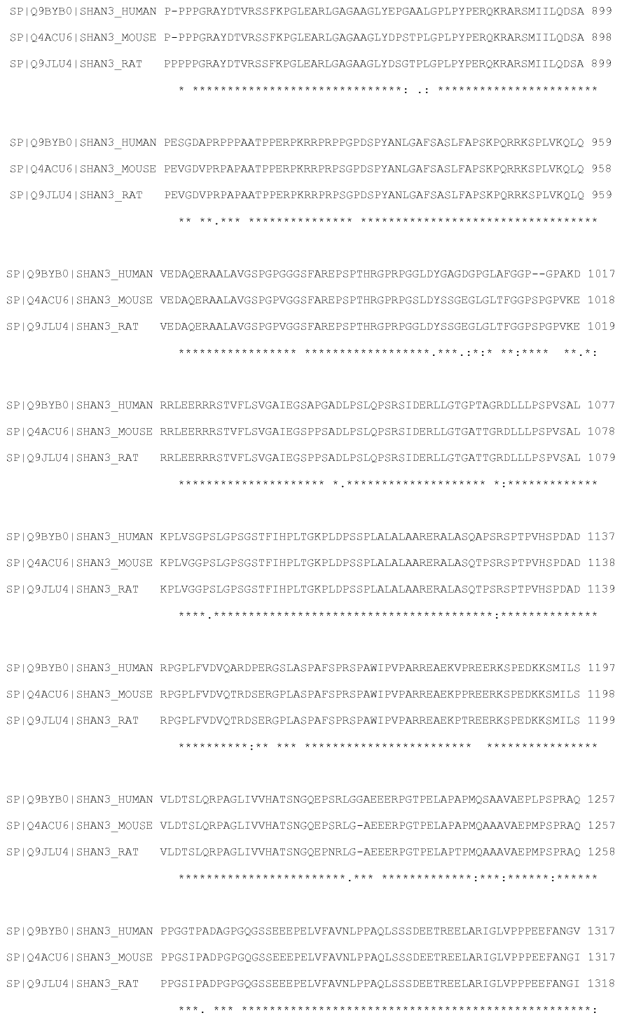

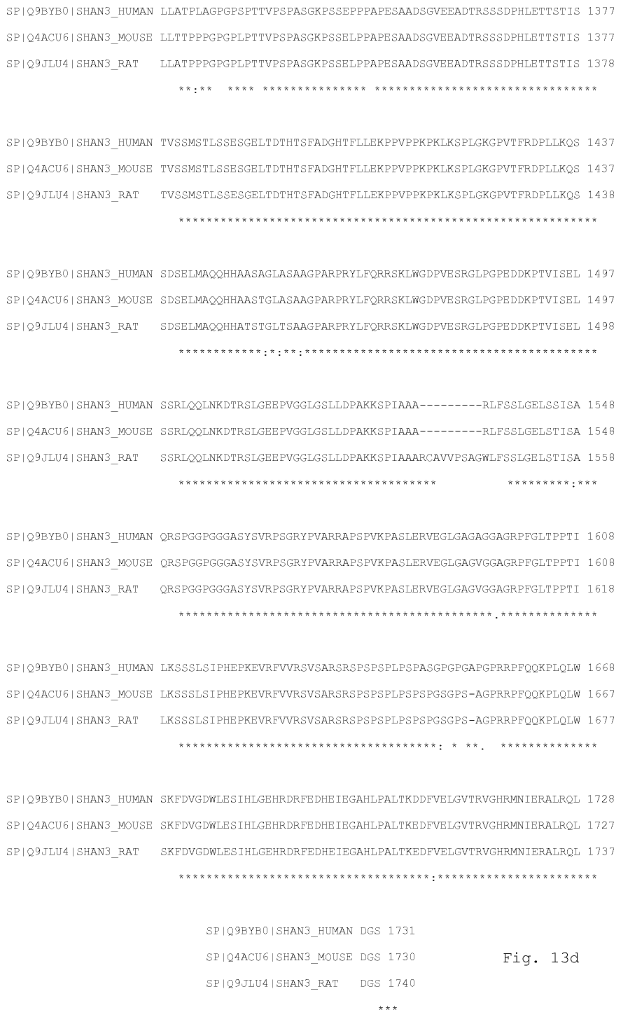

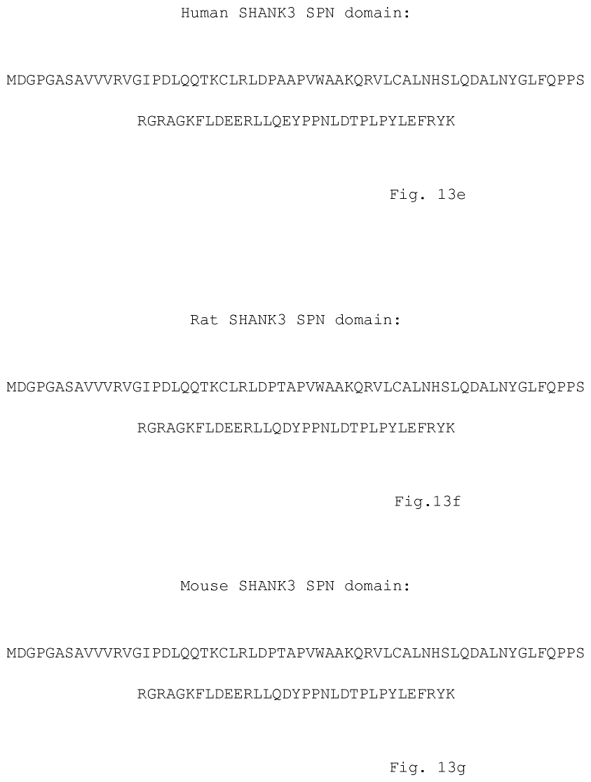

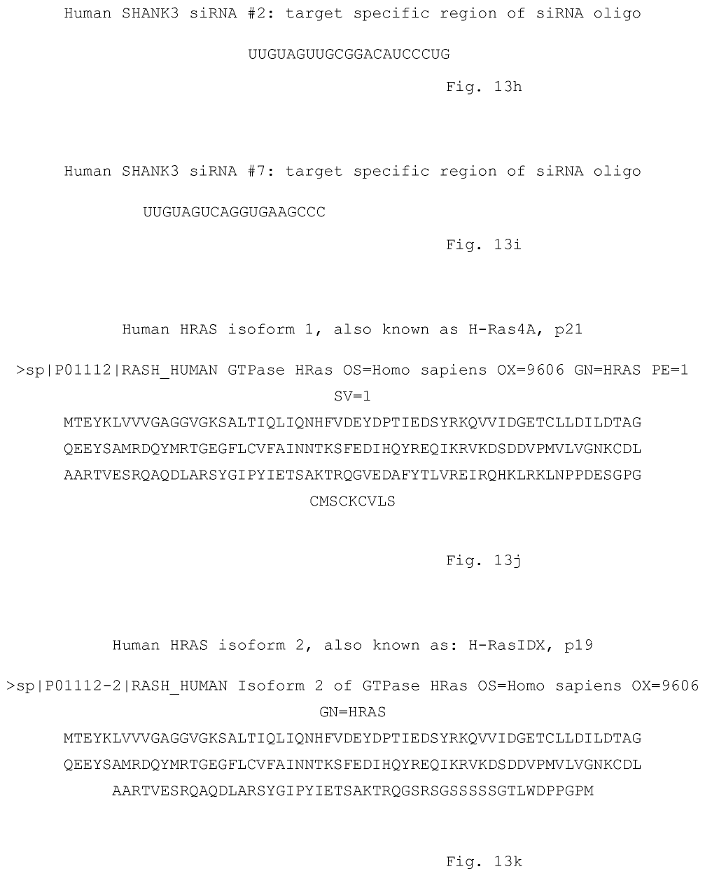

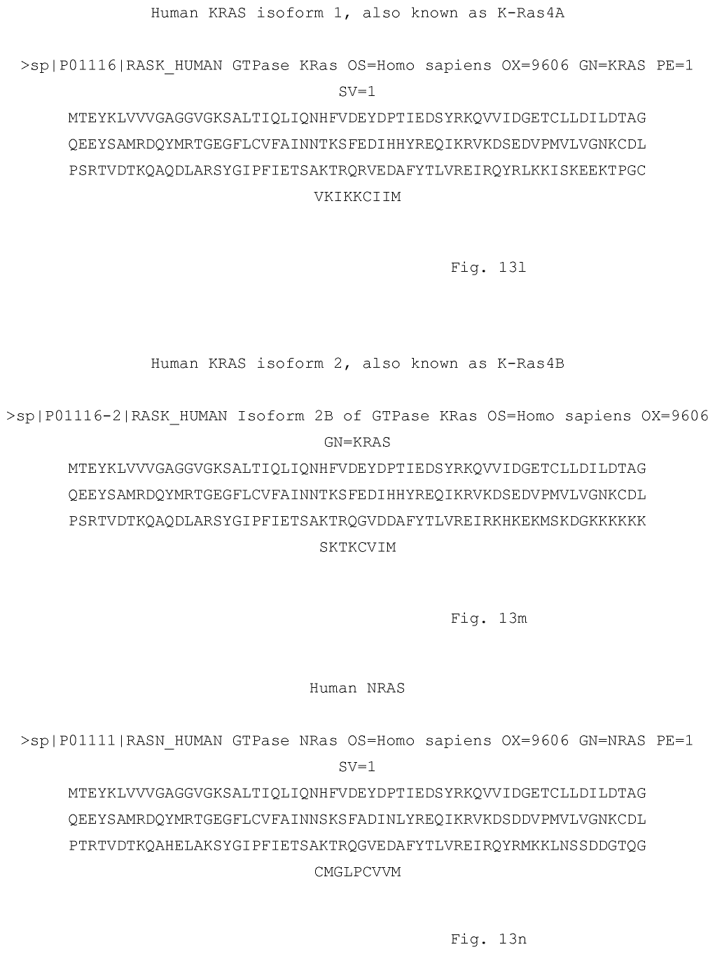

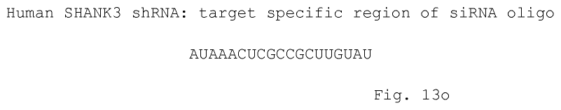

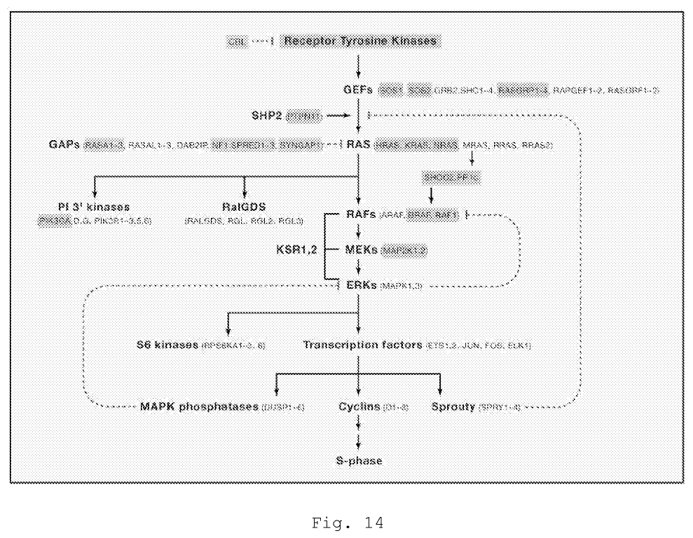

The inventors have determined the three-dimensional structure of the SHANK3 SPN-ARR which revealed that SHANK3 SPN belongs to Ras-association (RA) family of Ubl domains and interacts with several Ras and Rap proteins in their active and wild type forms¹³ (FIGS. 1a and b). To validate the ability of SHANK3-KRAS protein-protein interaction in cells, fluorescently tagged KRAS4bG12V and SHANK3 or SPN domain was only expressed in cells and performed RFP and GFP pull-downs. Both SHANK3 full length and SHANK3 SPN domain co-precipitated with active (G12V mutant) form of KRAS4b (FIGS. 1c, and d). The structure of SHANK3 SPN domain shows the presence of consensus RAS-recognizing positive charges (residues R12 and K22) in the β1 and β2 strands of SHANK3 (FIG. 1b). In this model, position of the SHANK3 SPN R12 side-chain is optimal for forming an ion-pair with E37 of KRAS and K22 with D38 or R40 of KRAS (FIG. 1b). Thus, introducing R12E/K22D charge reversal double mutation in SHANK3 SPN would disrupt KRAS binding. According to the results, the R12E/K22D mutation within SPN domain abolished SHANK3 SPN association with active (G12V mutant) KRAS in pull-downs (FIG. 1d). In addition, FLIM-FRET measurements indicated an interaction between active KRAS4b and SHANK3 SPN in cellular milieu (FIG. 1e)

### Example 2. SHANK3 Localizes to Membrane with Oncogenic KRAS

### Materials and Methods

Cell Lines and Cell Culture

MIA PaCa-2 (human pancreatic carcinoma, ATCC) cells were grown in DMEM (Dulbecco's modified Eagle's medium, Sigma-Aldrich) supplemented with 10% FBS and 2 mM L-glutamine. Cells were routinely tested for mycoplasma contamination.

Microscopy

MIA PaCa-2 cells were plated on glass-bottom dishes (MatTek corporation), previously coated with fibronectin and collagen overnight at 4° C., and transfected with GFP-tagged SPN WT or SPN R12E/K22D using Lipofectamine® 3000 (Invitrogen) for 24h. Cells were then fixed with 4% PFA in phosphate buffer saline (PBS) for 10 min at room temperature (RT) and washed with PBS. Imaging was performed with structure illumination microscopy (SIM) (DeltaVision OMX v4, GE Healthcare Life Sciences).

### Results

KRAS association with the plasma membrane is required for its signaling activity (16). Importantly, the KRAS fraction in the plasma membrane correlates with activation of the MAPK pathway and subsequent cellular proliferation (16). Previous results have shown that SHANK3 predominantly localizes to the periphery of the cell in actin-rich membrane ruffles (13). Thus, it was sought to explore the subcellular localization of SHANK3 in KRAS mutant cells using structure illumination microscopy (SIM). In the absence of suitable reagents to detect endogenous SHANK3 with immunofluorescence, SHANK3-GFP in KRAS-mutant (KRAS^{G12C}) MIA Paca-2 was expressed in pancreatic cancer cells. As expected, wild-type SHANK3 localized to the plasma membrane (FIG. 2). Importantly, the Ras binding deficient R12E/K22D mutant of SHANK3 localized less to the plasma membrane indicating that binding to active Ras is required for SHANK3 membrane localization (FIG. 2).

### Example 3. SHANK3 Inhibits Oncogenic RAS-ERK Signaling in Cells

### Materials and Methods

Cells and Cell Culture

MIA PaCa-2 (human pancreatic carcinoma, ATCC) and HCT116 (human colorectal carcinoma, ATCC) cells were grown in DMEM (Dulbecco's modified Eagle's medium, Sigma-Aldrich) supplemented with 10% FBS and 2 mM L-glutamine. All cells were routinely tested for mycoplasma contamination.

Western Blot Analyses

HCT116 cells were grown on 6-well plates overnight and transfected with GFP-tagged control (GFP only), SPN WT or SPN R12E/K22D using Lipofectamine® for 3000 (Invitrogen) 24 h as described above. Cells were washed with phosphate buffer saline (PBS), scraped in lysis bufferand samples were processed by western blot as described above. Following primary antibodies were used: phopho-ERK (Cell Signaling, 4370S) ERK (Cell Signaling, 91025), GAPDH (Hytest, 5G4-6C5) and GFP (Abcam, ab1218).

Immunofluorescent Staining and Imaging of ERK Localization

MIA PaCa-2 cells were grown on coverslips overnight and transfected with GFP-tagged SPN WT or SPN R12E/K22D using Lipofectamine® for 3000 (Invitrogen) 24 h as described above. Cells were then fixed with 4% PFA in PBS for 10 min at room temperature (RT), washed with PBS and permeabilized with 0.5% Triton-X-100 in PBS for 10 min at RT. PFA was quenched by incubating with 1 M Glycine for 30 min at RT. Cells were stained with the primary antibodies diluted in PBS (1:100) for 30 min at RT. Cells were then washed and incubated with Alexa-conjugated secondary antibodies (1:300), Phalloidin-Atto 647N (1:400) and 4′6-diamidino-2-phenylindole (DAPI, nuclei staining, 1:10000) diluted in PBS for 30 min at room temperature. Finally, cells were washed and imaging was performed with 3i spinning disk confocal (Marianas spinning disk imaging system with a Yokogawa CSU-W1 scanning unit on an inverted Carl Zeiss Axio Observer Z1 microscope, Intelligent Imaging Innovations, Inc., Denver, USA). Samples were either imaged right away or stored at +4° C. in dark until imaging.

### Results

Active KRAS stimulates downstream signaling pathways, especially the RAF/MEK/ERK pathway, to induce cell proliferation. Thus, it was sought to determine whether SHANK3 regulates KRAS signaling. To test this, the phosphorylation levels of ERK1/2 and AKT were studied in KRAS mutant (KRAS^{G12C}) HCT116 cells with low endogenous SHANK3 expression levels. Overexpression of SHANK3 SPN WT, but not R12E/K22D mutant, in HCT116 colon cancer cells led to decreased phosphorylation of ERK1/2 (Thr202/Tyr204) (FIG. 3a and b). Upon stimulation and activation ERK1/2 translocates to the nucleus, a key step in the transmission of ERK1/2 signals to regulate especially long-term consequences of ERK activation such as cell growth, (17). Overexpression of GFP-SHANK3 SPN WT, but not R12E/K22D mutant, in MIA Paca-2 pancreatic adenocarcinoma cells inhibited nuclear localization of ERK1/2 when compared to control GFP-transfected cells (FIGS. 3c and d). Taken together, these data show that SHANK3 specifically attenuates RAS/ERK-dependent signaling in KRAS-mutant cells.

### Example 4. SHANK3 Overexpression Inhibits KRAS-Induced Transformation and Tumorigenesis

### Materials and Methods

Cells and Cell Culture

HCT116 (human colorectal carcinoma, ATCC) and murine NIH/3T3 fibroblast were culture in DMEM supplemented with 10% FBS and 2 mM L-glutamine. NIH/3T3 fibroblast stably transformed with oncogenic KRAS (NIH/3T3 [KRAS^{V12}]) were culture in DMEM supplemented with 10% FBS, 2 mM L-glutamine and 1 μg/ml puromycin (18). All cells were routinely tested for mycoplasma contamination.

Colony Formation

NIH/3T3 wild-type or stably expressing K-rasG12V were used (18). Cells were transfected with GFP-tagged control plasmid or SPN WT using Lipofectamine® 3000 (Invitrogen) for 24 h as described above. Then, 1000 cells were seeded per well on a 12-well plate. The culture medium was replaced with fresh medium every second day. After 7 days, the medium was removed and cell colonies were stained with 0.2% Crystal Violet in 10% EtOH for 10 min at room temperature and washed with PBS. The average colony area percentage was calculated using the Colony area ImageJ plugin.

In Ovo Chicken Embryo Chorioallantoic Membrane (CAM) Assay

Fertilized chicken eggs were incubated as previously described (Beyer S J Biol. Chem. 2008). Shortly, the eggs were washed and the development was started by placing the eggs in 37° C. incubator. On day 3 of development, a small hole was made in the eggshell to drop the CAM. On developmental day 7, a plastic ring was placed on the CAM and one million either GFP-tagged control (GFP only), SPN WT or SPN R12E/K22D plasmid transfected HCT116 cells were implanted inside the ring in 20 μl of 50% Matrigel (diluted in PBS). After 4-5 days, tumors were imaged and dissected. The weight of dissected tumors were measured.

### Results

KRAS mutant cancers depend on the RAF-MEK-ERK cascade for initiation and maintenance of tumorigenic growth (REF). This prompted us to explore whether SHANK3 plays a functional role in KRAS-mutant cancer cells. First, murine NIH 3T3 cells were used that were stably transformed with oncogenic KRAS (NIH 3T3 [KRAS^{V12}]) and showed increased colony survival compared to non-transformed (NIH 3T3-) cells (FIGS. 4a and b). Overexpression of SHANK3 SPN decreased proliferationof NIH 3T3[K-Ras^{V12}] cells to a level comparable to non-transformed cells (FIGS. 4a and b). Importantly, SHANK3 SPN overexpression did not induce any significant changes in non-transformed NIH 3T3 cells (FIGS. 4a and b).

Furthermore, the in ovo chicken embryo chorioallantoic membrane (CAM) assay was used to follow tumor growth. Human HCT116 (KRAS^{G13D}) cells were transplanted onto the CAM membrane of fertilized eggs, resulting in rapid tumor formation. The tumor closely resembles cancer patient tumor as it contains extracellular matrix as well as stromal cells and extensive vasculature. The HCT116 tumor growth was significantly reduced upon overexpression of SHANK3 SPN WT, but not R12E/K22D mutant when compared to control transfected cells (FIGS. 4c and d). Together, these data demonstrate that SHANK3 suppresses KRAS-induced tumorigenesis in vitro and in ovo.

### Example 5. SHANK3 Suppresses KRAS-Induced Macropinocytosis

### Materials and Methods

Macropinosome Visualization and Quantification

MIA PaCa-2 cells were transfected with GFP-tagged control (GFP only), SPN WT or SPN R12E/K22D using Lipofectamine® 3000 (Invitrogen) for 24 h similarly as described above. Next day after transfections, cells were seeded on glassbottom dishes (Mattek), previously coated with fibronectin (10 μg/ml), to approximately 40-50% cell confluence and cultured overnight. Macropinocytosis was quantified as previously described (19). First, cells were washed twice with PBS to properly remove full cell culture medium and further, cells were incubated in serum-fee medium for 7-8 hours (serum-starvation). Macropinosomes were marked using a high-molecular-mass TMR-dextran uptake assay wherein the cells were incubated with TMR-dextran (Fina Biosolutions) in serum-free medium at a final concentration of 1 mg/ml for 40 min at 37° C. At the end of the incubation period, cells were rinsed five times in PBS and immediately fixed in 4% PFA 15 min at RT. Finally, cells were DAPI-stained for nuclei as described above ans cells were imaged using the 3i spinning disk confocal (Marianas spinning disk imaging system with a Yokogawa CSU-W1 scanning unit on an inverted Carl Zeiss Axio Observer Z1 microscope, Intelligent Imaging Innovations, Inc., Denver, USA). Macropinosomes inside the cells were analyzed using the analyze particles feature in Image) (National Institutes of Health).

### Results

Oncogenic KRAS stimulates macropinocytosis-mediated nutrient uptake in tumor cells¹⁹. Thus, it was tested if expression of SHANK3 in KRAS mutant cells could modify this oncogenic feature. Using a semiquantitative assay for micropinocytosis, that measures cellular uptake of high-molecular-mass dextran, robust uptake of dextran in KRAS mutant MIA PaCa-2 cells was observed as described previouslyl⁹. Importantly, overexpression of SHANK3 SPN WT, but not R12E/K22D mutant, significantly reduced macropinocytosiss in these cells (FIGS. 5a and b).

### Example 6. SHANK3 is Expressed at Low Levels in KRAS-Mutant Cancer

### Materials and Methods

Analysis of SHANK3 Expression

SHANK3 gene expression in human cancer and normal tissues was analysed using the publicly available FireBrowser gene expression viewer (http://firebrowse.org).

### Results

The finding that SHANK3 functions as an endogenous Ras-inhibitor in vitro and in vivo (FIG. 6a) prompted us to evaluate SHANK3 expression in clinical specimens. The TCGA database was utilized and found that SHANK3 mRNA was more abundant in normal solid tissue compared to primary tumors in lung and pancreatic cancer, cancer types where high frequency of KRAS mutations are a hallmark (FIG. 6b). Several other cancer types, such as head and neck, kidney, prostate, stomach and thyroid cancer showed no significant difference in SHANK3 mRNA levels between normal solid tissues and primary tumors (FIG. 6b). Thus, intriguingly, SHANK3 remained expressed at low levels in the majority of the tumors and was not fully lost, unlike many established tumor suppressors.

### Example 7. SHANK3 Silencing Inhibits Proliferation and Growth of KRAS-Mutant Cells

### Material and methods

Transient siRNA Transfections to Silence SHANK3 Expression

Cells were seeded on a 6-well plate a day before transfection and silenced the next day when cell confluence was approximately 30%. SiRNA silencing was performed using 50-100 nM siRNA (siRNA targeting SHANK3 or negative control siRNA) and Lipofectamine® RNAiMAX Reagent (Thermo Fisher Scientific Inc) according to manufacturer's instructions. Cells were silenced by changing culture medium to 1 ml of OptiMEM per well and by adding 500 ∥l of transfection mix, containing siRNA (final concentration 50-100 nM siRNA of targeting SHANK3 or 50-100 nM negative control siRNA) and RNAiMAX transfection reagents diluted in OptiMEM, dropwise on cells. Cells were incubated with transfection mix overnight and next day the silenced cells were used for experiments. The siRNAs targeting human SHANK3 were SMARTpool ON-TARGETplus Human SHANK3 siRNA (Cat. No. L-024645-00, Dharmacon), Individual Human SHANK3 siRNA_2 (Cat. No. S100717710 Hs_SHANK3_2 siRNA, Qiagen, sequence FIG. 7h and SEQ ID Nos 4) and ON-TARGETplus Human SHANK3 siRNA (J-024645-07 Dharmacon, sequence FIGS. 7i and SEQ ID Nos 5). SiRNAs used as controls were Allstars negative control siRNA (Qiagen, Cat. No. 1027281) and ON-TARGETplus Non-targeting Pool (Dharmacon, Cat. No. D-001810-10-05).

Western Blot Analyses

Cells were washed with PBS, scraped in lysis buffer and samples were processed by western blotting as described above. Following primary antibodies were used: SHANK3 (Santa Cruz, sc-30193) and GAPDH (Hytest, 5G4-6C5).

Proliferation Assay

Next day after silencing cells, 500 silenced cells were seeded per well in a 96-well plate in full culture medium. Proliferation was measured for 5-6 days with IncuCyte S3, 10× objective. Wells were imaged every two hours (brightfield and green phase; acquisition time 300 ms). Culture medium containing was changed every 2-3 days. Analysis was performed using IncuCyte S3 software. Analysis definition was set using the following parameters; segmentation (background-cells), cleanup (hole fill), filters (area, eccentricity, mean intensity, integrated intensity). A mask was set to the best fit of cell confluence to quantify cell area.

To perform a proliferation screen in several additional cancer cell lines, cells were plated in 96 wells (5000-10000 cells/well depending on the growth rate of control cells). Next day cells were silenced and proliferation rate was measured for 4-5 days with IncuCyte S3, 10× objective. Analysis was performed using IncuCyte S3 software as described above.

Colony Formation Assay

Next day after silencing cells, 125 or 250 cells were seeded per well on a 6-well plate in full medium. Medium was changed every 2-3 days and assay was ended after 10-14 days. Colonies were fixed with 4% PFA in PBS for 15 min and washed with PBS. Then, wells were stained with crystal violet for 15 min and washed with PBS. Plates were scanned and analyzed using an ImageJ plugin previously described by Guzman et al. 2014 (20).

3D Organoid Formation Assay

3D organoid formation was measured by a previously described method (Harma et al 2010) in where cells are embedded between two layers of matrigel. Angiogenesis 96-well μ-plate's (Ibidi GmbH) inner well was coated with 10 μl of 50% ECM (1:1 Matrigel:full cell culture medium, MTG stock 9 mg/ml). Plate was centrifuged at 200G for 20 min and incubated at +37° C. for 1 hour. Cells were silenced one day prior to seeding and then, wells were filled with 20 μl of cell suspension (500 cells per well) in 25% ECM (1:4 Matrigel: full cell culture medium). Plate was centrifuged at 100 G for 10 min and cells incubated at 37° C. for 4 h or overnight. Wells were filled with full cell culture medium and organoid formation was measured for 6-7 days with IncuCyte S3, 10× objective. Wells were imaged every two hours (Phase+brightfield and green phase, acquisition time 300 ms). Culture medium was changed every 2-3 days. Analysis was performed using IncuCyte S3 software. Analysis definition was set using the following parameters; segmentation (background-cells), cleanup (hole fill); filters (area, eccentricity, mean intensity, integrated intensity). A mask was set to the best fit of cell confluence to quantify cell area.

### Results

Next, it was sought to investigate what would be the outcome of depletion the low endogenous SHANK3 in KRAS-mutant cancer cells in vitro. Unexpectedly, SHANK3-silencing robustly reduced 2D proliferation and colony growth of PANC-1 pancreatic adenocarcinoma cells (KRAS^{G12D}) (FIGS. 7a and d) as well as A549 lung adenocarcinoma cells (KRAS^{G12S}) (FIGS. 7b and e) but only modest effect on proliferation of KRAS wild-type BxPC-3 pancreatic cancer cells (FIG. 7c). Accordingly, SHANK3-silenced PANC1 cells were not able to grow as 3D organoids in Matrigel whereas control cells formed organoids within 6 days (FIGS. 7f and g).

Further, multiple additional KRAS-mutant or KRAS WT cancer cell lines were screened in 2D proliferation assay. Accordingly, SHANK3-silencing reduced proliferation in PDAC (Panc10.05, AsPC-1, YAPC, SW1990, Su86.86, PaTu8902), LUAD (H441) and CRC (SW620, HCT-115, HCT-116) cell harboring distinct activating KRAS-mutations (FIG. 7h).

Taken together, these results indicate that loss of endogenous SHANK3 severely compromises proliferative capacity of cells containing an oncogenic KRAS mutation.

### Example 8. SHANK3 Silencing Induces Mapk Pathway Hyperactivation and Loss of Cell Viability in KRAS-Mutant Cells

### Materials and Methods

Effector-Recruitment FRET Assay

HEK293 cells were first silenced for control siRNA or SHANK3 targeting siRNA 48 hours, and then, seeded on a 6-well plate with glass coverslips, and plasmid-transfected with the donor alone (mGFP-tagged KRASG12V construct) in control samples, or together with the acceptor mRFP-RBD in C-Raf-RBD-recruitment FRET experiments. After 48 h of plasmid-transfection, coverslips were fixed with 4% PFA/PBS for 15 min and then washed with PBS, and coverslips were mounted with Mowiol 4-88 (Sigma Aldrich) on microscope slides. The mGFP fluorescence lifetime was measured using a fluorescence lifetime imaging attachment (Lambert Instruments, Groningen, Netherlands) on an inverted microscope (Zeiss AXIO Ovserver.D1, Jena, Germany) as previously described (20).

Western Blot Analyses

Cells were washed with PBS, scraped in lysis buffer and samples were processed by western blotting as described above. Following primary antibodies were used: SHANK3 (Santa Cruz, sc-30193), phopho-ERK1/2 (Thr202/Tyr204) (Cell Signaling, 4370S) ERK1/2 (Cell Signaling, 91025), phopho-AKT (Ser473) (Cell Signaling, 9271), AKT (Cell Signaling, 9272) GAPDH (Hytest, 5G4-6C5) and cleavedPARP1 (Abcam ab4830).

Annexin V-FITC/PI Flow Cytometry Assay

Two to three days after silencing, cells stained by eBioscienceT™ Annexin V-FITC Apoptosis Detection Kit. Cells were washed in PBS and resuspend in 200 μl of binding buffer (1×) (cell density 2-5×10⁵/ml). Then, 5 μL Annexin V-FITC was added to 195 μL cell suspension, mixed and incubated for 10 min at room temperature. Next, cells were washed in 200 μl of binding buffer (1×) and resuspend in 190 μl of binding buffer (1×). Then, 10 μl of propidium iodide (20 μg/mL) was added into cell suspension. FACS analysis was performed using BD LSRFortessa™ (BD Biosciences).

3D Organoid Formation Assay with AnnexinV

3D organoid formation was measured as described above. Cells were embedded between two layers of Matrigel and finally, covered with full cell culture medium containing 1:200 Annexin V (Annexin V-FITC Apoptosis Detection Kit, eBioscience™). Culture medium containing Annexin V was changed every 2-3 days.

Proliferation Assay of SHANK3 Silenced PANC-1 Cells Treated with ERK and MEK Inhibitors

Cells were seeded on a 96-well plate (2000-5000 cells/well) a day before transfection and silenced the next day when cell confluence was approximately 30%. Next day after silencing, medium was changed to full medium containing DMSO (control), Trametinib, Selumetinib (ADZ6244) or selective ERK1/2 inhibitor (SCH772984) in dose-dependent manner (concentration gradient: 0, 7.8 nM, 15.5 nM 31.3 nM 62.5 nM, 125 nM, 250 nM, 500 nM, 1 μM, 2 μM, 4 μM and 8 μM). Proliferation was measured using IncuCyte S3 as described above.

### Results

Next, it was studied the role of SHANK3 in KRAS-effector-recruitment by using a cell-based FRET assay (FIG. 8a). It was found that silencing of endogenous SHANK3 enhances RBD recruitment to active KRAS (KRAS^{G12V}) in HEK293 cells (FIG. 8b). This shows that the reduced in vitro and in ovo growth of KRAS-mutant cancer cells upon SHANK3-silencing could be a consequence of enhanced KRAS downstream signaling.

KRAS mutant lung and pancreatic cancer cell lines are sensitive to hyperactivation of the ERK that leads to RAS-ERK-dependent toxicity¹¹ and mouse models of RAS-induced tumors define a narrow range of oncogenic RAS signalling that is permissive to tumour formation with too much triggering growth arrest and too little signalling not supporting increased proliferation^{10,20}. Thus, RAS mutant cancer cells may require a mechanism to limit active ERK levels from reaching a lethal signalling threshold. Thus, it was investigated whether depletion of the low endogenous SHANK3 in KRAS-mutant pancreatic and lung cell lines could cause hyperactivation of ERK, producing a signalling intensity that leads to cell toxicity. Excitingly, silencing of SHANK3 induced a very strong 10-30-fold increase in ERK1/2 phosphorylation in PANC-1 pancreatic adenocarcinoma cells as well as in A549 lung adenocarcinoma cells (FIGS. 8c, d and f) whereas AKT activity showed variable non-significant changes (FIGS. 8c, d and g). Importantly, ablation of SHANK3 in KRAS wild-type BxPC-3 pancreatic cancer cells had no significant effect on ERK or AKT activity (FIGS. 8e, f and g). SHANK3-silencing induced the levels of PARP-1 cleavage in PANC-1 and A549 cells (FIGS. 8c, d and h), indicating that a loss of SHANK3 induces apoptosis in KRAS mutant cells.

Further, Annexin V-FITC/PI flow cytometry assay was used to analyze the population of apoptotic cells. A significant increase in apoptosis already two days after SHANK3-silencing in PANC-1 cells was observed (FIGS. 8i and j). Moreover, SHANK3-silenced PANC1 cells grown in 3D matrigel showed increasing numbers of AnnexinV positive apoptotic cells in time-dependent manner (FIGS. 8k and l) and consequently, the silenced cells failed to form proper organoids, as also seen in FIGS. 7f and g.

Finally, it was tested whether RAS hyperactivation-induced cell death triggered by inhibition SHANK3-silencing in KRAS mutated cells, could be rescued by inhibition of the MAPK pathways with ERK or MEK inhibitors. Indeed, treatment with Trametinib, Selumetinib (ADZ6244) or selective ERK1/2 inhibitor (SCH772984) in dose-dependent manner abolished a loss of cell viability induced by SHANK3-silencing in PANC-1 cells (FIG. 8m). At the low concentrations used, the inhibitors had no significant effect on the viability of the control silenced cells (FIG. 5m). These data indicate that reduced cell viability caused by depletion of SHANK3 in KRAS-mutant cells is linked to increased phosphorylation of ERK1/2.

Taken together, these results show that knockdown of SHANK3 induces MAPK/ERK hyperactivation and a loss of cell viability in cells containing an oncogenic KRAS mutation (FIG. 9).

### Example 9. Loss of SHANK3 Impairs Growth of KRAS-Mutant Tumors

### Materials and Methods

In Ovo Chicken Embryo Chorioallantoic Membrane (CAM) Assay

Fertilized chicken eggs were incubated and cells were processed as described above, with exception that one million control or SHANK3 targeting siRNAtransfected PANC-1, A549 or BXPC-3 cells were implemented per egg.

Subcutaneous Tumor Xenografts in Nude Mice

For subcutaneous (s.c.) tumors, 4.5 million siCTRL or siSHANK3 (SHANK3 siRNA_7) treated PANC-1 pancreatic cancer cells (1.5 days after silencing) were injected s.c. in 100 μl (50% Matrigel, 50% PBS) to the flank of 6-8 weeks old female Nude mice (Hsd:AthymicNude-Foxnlnu, Envigo). Tumor growth was followed by measurement of tumor diameter with caliper 1-3 times per week. In order to determine tumor volume by external caliper, the greatest longitudinal diameter (length) and the greatest transverse diameter (width) were determined. Tumor volume based on caliper measurements were calculated by the modified ellipsoidal formula: tumor volume=½ (length×width×width). Tumor volume for flat tumors (non-ellipsoidal shape) were calculated by modified formula: tumor volume=½ (length×width×⅓ width). Mice were sacrificed after 20 days, and tumors were dissected, weighted, and fixed in 10% formalin. All animal experiments were ethically assessed, authorised by the National Animal Experiment Board and in accordance with The Finnish Act on Animal Experimentation (Animal licence numbers ESAVI/9339/2016 and ESAVI/37571/2019).

HE Staining and Analysis of Tumors

Formalin-fixed, paraffin-embedded tissue samples were cut to 4 μm sections, deparaffinized and rehydrated with standard procedures, and stained with conventional hematoxylin-eosin (HE). Stained samples were imaged with Pannoramic P1000 Slide Scanner (3DHISTECH Ltd) and analysed using QuPath software.

IHC Staining and Analysis of Tumors

Formalin-fixed, paraffin-embedded tissue samples were cut to 4 μm sections, deparaffinized and rehydrated with standard procedures. For immunohistochemistry (IHC) of CAM tumors, heat-mediated antigen retrieval was done in citrate buffer (pH 6 for cleaved caspase-3, pH 9 for Ki-67). Sections were washed with washing buffer (Tris-HCl0.05 M pH 7.6, 0.05% Tween20), blocked for endogenous hydrogen peroxide activity, and incubated with Normal Antibody Diluent (NABD; Immunologic, BD09-125). Sections were then incubated with a Ki-67 antibody (Millipore AB9260, diluted 1:1000) or Cleaved Caspase-3 (Asp175) antibody (#9664, Cell Signaling Technology, clone 5A1E, diluted 1:500) for 1 h. After washes, samples were incubated 30 min with BrightVision Goat anti-Rabbit HRP (Immunologic DPVR110HRP) secondary antibody, and washed again. After washes, DAB solution (DAKO K3468) was added for 10 sec followed by washing. After counterstain with Mayer's HTX, slides were dehydrated, cleared in xylene and mounted in Pertex. Stained samples were imaged with Pannoramic P1000 Slide Scanner (3DHISTECH Ltd), and analysed with QuantCenter software with NuclearQuant quantification module (3DHISTECH Ltd).

### Results

To consolidate the in vitro findings with in vivo tumor growth, pancreatic and lung cancer CAM xenograft models were employed. In line with the in vitro results, SHANK3-silencing significantly abrogated the formation and growth of PANC-1 and A549 KRAS mutant tumors (FIG. 10a-f). This was evident in both a decrease in tumor weight (FIGS. 10a and d) and in number of Ki-67 (proliferation marker) positive cells (detected with immunohistochemical (IHC) staining) when compared to control tumors (FIGS. 10b, c, e and f). However, the growth of KRAS wild-type BxPC-3 tumors was not aberrantly affected upon SHANK3 depletion (FIG. 10g-i).

To confirm whether decreased tumor size observed in the in vivo CAM assays following SHANK3 silencing was due to initiation of apoptosis, the tumor samples were analyzed by IHC using the apoptosis marker cleaved caspase-3. Notably higher cleaved caspase-3 staining was observed in SHANK3-silenced KRAS mutant A549 tumors compared to control tumors (FIG. 10j, k). Accordingly, the residual SHANK3-silenced PANC-1 tumors showed increased cleaved caspase-3 staining (FIG. 10l).

Next, subcutaneous (s.c) xenograft model of pancreatic cancer in mice was established. Accordingly, SHANK3-silencing robustly impaired the formation and growth of PANC-1 xenografts (FIG. 10m-p). We observed a significant decrease in tumor volume as well as in number of tumor cells detected by HE staining when compared to control tumors (FIG. 10m-p).

Cumulatively, these results show that depletion of endogenous SHANK3 specifically triggers apoptosis and impairs in vivo proliferation and growth of KRAS mutant cancer.

### Example 10. Inducible Depletion of SHANK3 In Vivo Inhibits Tumor Growth

### Materials and methods

Doxycycline-Inducible shSHANK3 PANC-1 Cell Line

SMART lentiviral shRNA vectors for doxycycline-inducible suppression of human SHANK3 gene expression were purchased from Dharmacon as viral particles (Dox-inducible SMARTvector shSHANK3, V3SH7669-228381856, Dharmacon). Packaged lentiviruses were then applied to PANC-1 cells in the presence of polybrene (8 μg/ml, TR-1003-G, Sigma-Aldrich) and incubated overnight, and then selected using puromycin (5 μg/ml, #15205, Sigma-Aldrich). Single-cell clones were created by screening for high induction efficacy (bright tRFP positive clones after dox-induction; indicative of SHANK3 shRNA expression).

2D Proliferation

To measure proliferation, cells were seeded on a 96-well plate in full culture medium. Doxycycline induction (+dox; 2 μg/ml) was started 24 hours postplating. Proliferation was measured for 6 days using the IncuCyte S3 Live-Cell Analysis system (10× objective) as described above. Culture medium including doxycycline (+dox) (or −dox) was changed every second day. Analysis was performed using IncuCyte S3 software.

3D Spheroid Growth

To study 3D growth, PDAC spheroids were established in Matrigel as described above. Then, SHANK3-depletion was induced by doxycycline in established spheroids. Both doxycycline (+dox; 2 μg/ml) and AnnexinV (1:200, Annexin V-FITC Apoptosis Detection Kit, eBioscience™) were added to spheroids at day 5 and spheroid growth was followed for 10 days. Culture medium containing doxycycline (or -dox) and AnnexinV was changed every second day. Analysis was performed using IncuCyte S3 software.

### In Vivo Xenograft Model

For subcutaneous tumor model, six- to eight-week-old female athymic Nude mice (Hsd:Athymic Nude-foxn1nu, Envigo, France) were injected in the flank with 5×10⁶ human PANC-1 doxycycline-inducible SHANK3 shRNA-containing cells (pool of clones 4S and 1C) resuspended in 100 ul PBS with 50% Matrigel. When tumors reached an average mean volume of 100 mm³, the mice with similarly sized tumors were blindly randomized into cohorts. Then, mice were fed normal chow (control group) or doxycycline-containing chow (SHANK3 depletion induced) daily. In addition, mice received two intraperitoneal injections of PBS or doxycycline (80 mg/kg of body weight). Successful induction of SHANK3 shRNA expression was confirmed by IVIS imaging (tRFP expression after dox-induction; indicative of SHANK3 shRNA expression). Tumors were measured with calipers twice a week and tumour volume was calculated according to the formula V=(π/6) (d1×d2)\hat{ }3/2, where d1 and d2 are perpendicular tumour diameters. Mice were sacrificed at day 74 post-engraftment, and tumors were dissected. Animal studies were ethically performed and authorised by the National Animal Experiment Board and in accordance with The Finnish Act on Animal Experimentation (Animal licence number ESAVI-9339-04.10.07-2016).

### Results

This work demonstrates that SHANK3 suppression using two independent RNAi oligonucleotides, thus removing an endogenous inhibitor of KRAS signaling, triggers a seemingly cytotoxic level of ERK activity that results in reduced cell proliferation, induction of apoptosis and impaired tumor growth in KRAS mutant xenograft models. To further validate these findings, inducible shRNA-mediated depletion of SHANK3 in KRAS-mutant cancer cell lines was established. We stably infected the KRASG12D-mutant PANC-1 cells, using a lentivirus shRNA vector encoding a human SHANK3 shRNA driven by a doxycycline-inducible promoter (Doxinducible SMARTvector shSHANK3 8319506, Dharmacon) and generated single cell clones and a pool of two clones (combination of clones 4S and 1C).

To validate single cell clones, we analyzed SHANK3 gene expression (mRNA levels) in control (−dox) or doxycycline-induced (+dox; 72h) shSHANK3expressing PANC-1 clones by qRT-PCR and observed the loss of SHANK3 after doxycycline (+dox) induction (FIG. 11a and b). In line with the siRNA-silencing results, doxycycline-induced depletion of SHANK3 in PANC-1 cells promoted an increase in ERK1/2 phosphorylation (indicative of ERK activation) and enhanced PARP1 cleavage (indicative of apoptosis) (FIG. 11c). Further, we investigated the kinetics of ERK activation and apoptosis and observed a time-dependent increase in the levels of both ERK phosphorylation and cleaved-PARP1 (11d).

Accordingly, doxycycline-induced SHANK3 depletion in PANC-1 cells dramatically reduced 2D proliferation measured by IncuCyte S3 Live-Cell Analysis system (FIGS. 11e and f). Most notably, induction of SHANK3-silencing dampened the growth of established 3D spheroids and was accompanied by a significant increase in AnnexinV-positive regions within the spheroids over time (FIG. 11g-i).

Next, it was evaluated whether SHANK3 was also essential for maintenance of the tumorigenic growth of established KRAS-mutant tumors, by employing a subcutaneous xenograft model in Nude mice. Doxycycline-inducible SHANK3 shRNA-containing PANC-1 cells were subcutaneously implanted and tumors were allowed to grow. When the average volume of the tumors reached about 100 mm³, mice were divided into a control group and a therapy group which were fed normal chow (−dox) or doxycycline-containing chow (SHANK3 depletion induced; +dox), respectively, and tumor growth was observed for 26 days (FIG. 11j). Intriguingly, doxycycline-treated tumors showed dramatic impairment of tumor growth (FIG. 11k-m). These results indicate that an inducible depletion of endogenous SHANK3 is effective in blocking KRAS-mutant tumor growth in vivo.

Targeting SHANK3 to induce RAS pathway hyperactivation-induced apoptosis represents a conceptually novel therapeutic approach for the treatment of KRAS-driven cancers (FIG. 12).

### Statistical Analyses

Sample size for the studies was chosen according to previous studies in the same area of research. GraphPad program was used for all statistical analyses. Normal distribution of the data was tested with D'Agostino & Pearson omnibus normality test. Student's t-test (unpaired, two-tailed) with Welch's correction was used for two groups when normality could be confirmed. Nonparametric Mann-Whitney U-test was used when two non-normally distributed groups were compared or when normality could not be tested [due to a too small data set (n<8)]. One-way ANOVA with Holm-Sidak's multiple comparison test was used when comparing more than two normally distributed groups. Kruskal-Wallis non-parametric test with Dunn's multiple comparison test was used when comparing more than two non-normally distributed groups. Data are presented in column graphs with mean±standard error of mean (s.e.m) or mean±standard deviation (s.d) and P-values. Individual data points per condition are shown and n-numbers are indicated in figure legends. P-values less than 0.05 were considered to be statistically significant.

It is clear for a person skilled in the art that, as technology advances, the inventive concept can be implemented in various ways i.a. as described below. The invention and its embodiments are not limited to the examples described above but may vary within the scope of the claims.

The high, unmet need for improved therapies for multiple cancers has rendered oncology one of the major focus areas for pharmaceutical and biotechnology companies. As a result, the global cancer market represents the most dynamic pharmaceutical markets worldwide, characterized by a changing commercial landscape and a high degree of innovation. Before the present invention, there were no leading contenders for the treatment of PDAC on the market, and the most recent clinical trials with RAS-pathway inhibitors have demonstrated little or no significant improvement over standard therapies. Moreover, the main strategies for developing anti-RAS therapies (in ongoing trials or discontinued products) have all focused on reducing downstream RAS signaling without much success. Therefore, the market potential for a novel drug that can combat PDAC directly or enhance the effectiveness of chemotherapeutic drugs through an innovative approach is huge. The expected global compound annual growth rate (CAGR) of pancreatic cancer therapeutics, largely driven by the considerable increase in the predicted number of patients, is 8.1% in the forecast period (2018-2025).

In addition to PDAC, approach of the present invention to be attractive for the lung adenocarcinoma (CAGR: 7.6%; USD 22.6 billion in 2022) and CRC (CAGR: 1.9%; USD 9.8 billion in 2024) therapeutic markets, especially considering that a large proportion of the patients (31% for lung and 45% for colorectal cancer) within these groups have KRAS mutant cancers.

There are also significant societal benefits of the present invention. KRAS mutations are found in ˜98% of all PDACs, ˜31% of lung adenocarcinomas and ˜45% of all CRCs, three of the top four neoplasms accounting for cancer patient deaths. In 2018 alone, PDAC (128,000), lung cancer (388,000) and CRC (243,000) accounted for ˜40% of all cancer mortality cases in Europe²¹.

Although KRAS mutations are the most frequent oncogene aberrations in the above cancers, current treatments are limited to combined non-specific chemotherapy with conventional cytotoxic drugs in KRAS-driven lung cancer or palliative therapy in PDAC, the most KRAS-addicted of all cancers, offering only marginal survival benefits for patients. In addition, KRAS mutant CRCs are currently lacking targeted therapy options available to KRAS wt CRCs (anti-EGFR). As such, complete resection followed by adjuvant treatment remains the only realistic curative option for PDAC patients, and only in those with a good performance status and a suitably accessible tumour. Therefore, introducing a novel and more specific therapy for PDAC, and other KRAS-addicted cancers according to the invention, has the potential to transform the lives of hundreds of thousands patients affected annually and will have huge economic benefits for society. Thus, the present invention provides a very significant advantage for overcoming tumorous diseases.

### REFERENCES

1. Stephen, A. G., Esposito, D., Bagni, R. K. & McCormick, F. Dragging ras back in the ring. Cancer Cell 25, 272-281 (2014).
2. Hobbs, G. A., Der, C. J. & Rossman, K. L. RAS isoforms and mutations in cancer at a glance. J. Cell. Sci. 129, 1287-1292 (2016).
3. Waters, A. M. & Der, C. J. KRAS: The Critical Driver and Therapeutic Target for Pancreatic Cancer. Cold Spring Harb Perspect Med 8, (2018).
4. Cox, A. D., Fesik, S. W., Kimmelman, A. C., Luo, J. & Der, C. J. Drugging the undruggable RAS: Mission possible? Nat Rev Drug Discov 13, 828-851 (2014).
5. Bryant, K. L., Mancias, J. D., Kimmelman, A. C. & Der, C. J. KRAS: feeding pancreatic cancer proliferation. Trends Biochem. Sci. 39, 91-100 (2014).
6. Karnoub, A. E. & Weinberg, R. A. Ras oncogenes: split personalities. Nat. Rev. Mol. Cell Biol. 9, 517-531 (2008).
7. Goetz, E. M., Ghandi, M., Treacy, D. J., Wagle, N. & Garraway, L. A. ERK mutations confer resistance to mitogen-activated protein kinase pathway inhibitors. Cancer Res. 74, 7079-7089 (2014).
8. Cisowski, J., Sayin, V. I., Liu, M., Karlsson, C. & Bergo, M. O. Oncogene-induced senescence underlies the mutual exclusive nature of oncogenic KRAS and BRAF. Oncogene 35, 1328-1333 (2016).
9. Unni, A. M., Lockwood, W. W., Zejnullahu, K., Lee-Lin, S.-Q. & Varmus, H. Evidence that synthetic lethality underlies the mutual exclusivity of oncogenic KRAS and EGFR mutations in lung adenocarcinoma. Elife 4, e06907 (2015).
10. Pershing, N. L. K. et al. Rare codons capacitate Kras-driven de novo tumorigenesis. J. Clin. Invest. 125, 222-233 (2015).
11. Unni, A. M. et al. Hyperactivation of ERK by multiple mechanisms is toxic to RTK-RAS mutation-driven lung adenocarcinoma cells. Elife 7, (2018).
12. Chong, C. R. & Jänne, P. A. The quest to overcome resistance to EGFR-targeted therapies in cancer. Nat. Med. 19, 1389-1400 (2013).
13. Lilja, J. et al. SHANK proteins limit integrin activation by directly interacting with Rap1 and R-Ras. Nat. Cell Biol. 19, 292-305 (2017).
14. Simanshu, D. K., Nissley, D. V. & McCormick, F. RAS Proteins and Their Regulators in Human Disease. Cell 170, 17-33 (2017).
15. Misale, S. et al. KRAS G12C NSCLC Models Are Sensitive to Direct Targeting of KRAS in Combination with PI3K Inhibition. Clin. Cancer Res. 25, 796-807 (2019).
16. Kim, D.-H. et al. Synthetic dsRNA Dicer substrates enhance RNAi potency and efficacy. Nat. Biotechnol. 23, 222-226 (2005).
17. Cui, W., Ning, J., Naik, U. P. & Duncan, M. K. OptiRNAi, an RNAi design tool. Comput Methods Programs Biomed 75, 67-73 (2004).
18. Jinek, M. et al. A programmable dual-RNA-guided DNA endonuclease in adaptive bacterial immunity. Science 337, 816-821 (2012).
19. Commisso, C. et al. Macropinocytosis of protein is an amino acid supply route in Ras-transformed cells. Nature 497, 633-637 (2013).
20. Li, S., Balmain, A. & Counter, C. M. A model for RAS mutation patterns in cancers: finding the sweet spot. Nat. Rev. Cancer 18, 767-777 (2018).
21. Ferlay, J. et al. Cancer incidence and mortality patterns in Europe: Estimates for 40 countries and 25 major cancers in 2018. Eur. J. Cancer 103, 356-387 (2018).

1. A SH3 and multiple ankyrin repeat domains 3 (SHANK3) inhibiting agent for use in preventing, treating or ameliorating a RAS-dependent cancer or diminishing the amount of RAS-dependent cancer cells, wherein said agent inhibits, depletes or diminishes the function of SHANK3.

2. SHANK3 inhibiting agent for use according to claim 1, wherein the SHANK3 inhibiting agent inhibits, diminishes or depletes an interaction or association of SHANK3 with a RAS isoform, thereby activating RAS-pathway.

3. SHANK3 inhibiting agent for use according to claim 1, wherein the RAS is KRAS, HRAS or NRAS.

4. SHANK3 inhibiting agent for use according to claim 1, wherein the RAS is KRAS encoded by a gene with one or more mutations in the KRAS gene located at a codon encoding amino acid residues at positions 5, 8, 9, 12, 13, 14, 19, 21, 22, 33, 36, 58, 59, 61, 62, 63, 68, 71, 72, 92, 117, 119, and/or 146 of SEQ ID NO: 8 or 9.

5. SHANK3 inhibiting agent for use according to claim 1, wherein the agent is a binding molecule specifically binding to SHANK3.

6. SHANK3 inhibiting agent for use according to claim 1, wherein said agent is an antibody, a nanobody, an affibody, an aptamer, a peptide or a small-molecule inhibitor.

7. SHANK3 inhibiting agent for use according to claim 1, wherein said agent inhibits SHANK3 gene expression.

8. SHANK3 inhibiting agent for use according claim 7, wherein said agent is selected from the group consisting of siRNA molecules, shRNA molecules, DsiRNA molecules, artificial miRNA precursors, and antisense oligonucleotides.

9. SHANK3 inhibiting agent for use according to claim 8, wherein the agent comprises a target-specific region comprising a polynucleotide having a nucleic acid sequence set forth in any one of SEQ ID NOs: 4, 5 and 17-35, or a sequence having at least 80% identity to the sequence set forth in any one of SEQ ID NOs: 4, 5 and 17-35 provided that the SHANK3 inhibiting activity of the agent is retained.

10. SHANK3 inhibiting agent for use according to claim 1, wherein said agent is a gene editing agent.

11. SHANK3 inhibiting agent for use according to claim 1, wherein said cancer involves an overactive RAS-MAPK.

12. SHANK3 inhibiting agent for use according to claim 1, wherein said cancer is pancreatic cancer, lung cancer, colorectal cancer, ovarian cancer, melanoma, urinary bladder carcinoma, thyroid carcinoma, hematopoietic malignancy, liver carcinoma, breast cancer, neuroblastoma, cervix adenocarcinoma, head and neck carcinoma, stomach cancer, biliary tract adenocarcinoma, angiosarcoma, malignant fibrous histiocytoma, or any other cancer that is RAS-dependent, RAS-driven or has a mutation upstream of RAS pathway.

13. A method for identifying a candidate compound for treatment of RAS dependent cancer, the method comprising:
i. contacting a SHANK3 polypeptide and a RAS polypeptide with a test compound,
ii. determining whether the test compound reduces binding between SHANK3 and RAS, and
iii. identifying the test compound as a candidate compound for treatment of RAS dependent cancers, if said binding is reduced by at least 10%.

14. The method according to claim 13, wherein the SHANK3 polypeptide has at least 80% identity to SEQ ID NO: 1, 2, 3, 11, 12, 13 or 36.

15. The method according to claim 13, wherein the RAS polypeptide has at least 80% identity to any one of SEQ ID NO: 6, 7, 8, 9, 10, 15 or 16.

16. The method according to claim 15, wherein said SEQ ID NO: 8 or SEQ ID NO: 9 comprises one or more amino acid substitutions at positions selected from the group consisting of positions 5, 8, 9, 12, 13, 14, 19, 21, 22, 33, 36, 58, 59, 61, 62, 63, 68, 71, 72, 92, 117, 119, and 146.

17. The method according to claim 16, wherein said SEQ ID NO: 8 or SEQ ID NO: 9 comprises one or more amino acid substitutions selected from the group consisting of G12A, G12C, G12D, G12R, G125, G12V, G13C, G13D, L19F, Q22K, D33E, A59G, Q61H, Q61L, Q61R, E62K, K117N, A146T, K5E, G13V, V14I, T58K, A59E, A59T, Q61E, Q61K, E63K, Y71C, A146V, A146P, I36M, R68M, R68S, and D92Y.

18. The method according to claim 13, wherein SHANK3 or RAS is labelled with a detectable label, and/or SHANK3 or RAS is immobilized on a surface.

19. The method of claim 13 wherein the test compound is selected in silico.

20. The method of claim 13, wherein the test result is verified in a cellular assay.

21. Use of SHANK3 for screening or identifying one or more candidate compounds for treatment of RAS-dependent cancer.

22. Use of an in silico model of SHANK3 for screening or identifying one or more candidate compounds for treatment of RAS-dependent cancer.

23. Use according to claim 21, wherein the SHANK3 has an amino acid sequence having at least 80% identity to SEQ ID NO: 1, 2, 3, 11, 12, 13 or 36.

24. A kit comprising an isolated SHANK3 polypeptide and an isolated RAS-isoform polypeptide, or domains thereof responsible for SHANK3-RAS binding.

25. Use of the kit according to claim 24 for screening or identifying one or more candidate compounds for treatment of RAS-dependent cancer.

26. A computer-based method for identifying or designing a candidate compound for treatment of RAS dependent cancer, the method comprising
i. providing a spatial structure of the RAS binding domain of SHANK3, wherein said domain comprises at least amino acids, corresponding to R12 and K22 in the in the human RAS binding domain of SHANK3 (SEQ ID NO. 11), in a computer
ii. generating a spatial structure of potential inhibitors in a computer, and
iii. selecting potential inhibitors having a structure which can bind at least one amino acid residue of said domain.


SEQUENCE LISTING (37 sequences)

SEQ ID NO: 1 - protein, 1731 aa, Homo sapiens
MDGPGASAVVVRVGIPDLQQTKCLRLDPAAPVWAAKQRVLCALNHSLQDALNYGLFQPPSRGRAGKFLDEERLLQEYPPNLDTPLPYLEFRYKRRVYAQNLIDDKQFAKLHTKANLKKFMDYVQLHSTDKVARLLDKGLDPNFHDPDSGECPLSLAAQLDNATDLLKVLKNGGAHLDFRTRDGLTAVHCATRQRNAAALTTLLDLGASPDYKDSRGLTPLYHSALGGGDALCCELLLHDHAQLGITDENGWQEIHQACRFGHVQHLEHLLFYGADMGAQNASGNTALHICALYNQESCARVLLFRGANRDVRNYNSQTAFQVAIIAGNFELAEVIKTHKDSDVVPFRETPSYAKRRRLAGPSGLASPRPLQRSASDINLKGEAQPAASPGPSLRSLPHQLLLQRLQEEKDRDRDADQESNISGPLAGRAGQSKISPSGPGGPGPAPGPGPAPPAPPAPPPRGPKRKLYSAVPGRKFIAVKAHSPQGEGEIPLHRGEAVKVLSIGEGGFWEGTVKGRTGWFPADCVEEVQMRQHDTRPETREDRTKRLFRHYTVGSYDSLTSHSDYVIDDKVAVLQKRDHEGFGFVLRGAKAETPIEEFTPTPAFPALQYLESVDVEGVAWRAGLRTGDFLIEVNGVNVVKVGHKQVVALIRQGGNRLVMKVVSVTRKPEEDGARRRAPPPPKRAPSTTLTLRSKSMTAELEELASIRRRKGEKLDEMLAAAAEPTLRPDIADADSRAATVKQRPTSRRITPAEISSLFERQGLPGPEKLPGSLRKGIPRTKSVGEDEKLASLLEGRFPRSTSMQDPVREGRGIPPPPQTAPPPPPAPYYFDSGPPPAFSPPPPPGRAYDTVRSSFKPGLEARLGAGAAGLYEPGAALGPLPYPERQKRARSMIILQDSAPESGDAPRPPPAATPPERPKRRPRPPGPDSPYANLGAFSASLFAPSKPQRRKSPLVKQLQVEDAQERAALAVGSPGPGGGSFAREPSPTHRGPRPGGLDYGAGDGPGLAFGGPGPAKDRRLEERRRSTVFLSVGAIEGSAPGADLPSLQPSRSIDERLLGTGPTAGRDLLLPSPVSALKPLVSGPSLGPSGSTFIHPLTGKPLDPSSPLALALAARERALASQAPSRSPTPVHSPDADRPGPLFVDVQARDPERGSLASPAFSPRSPAWIPVPARREAEKVPREERKSPEDKKSMILSVLDTSLQRPAGLIVVHATSNGQEPSRLGGAEEERPGTPELAPAPMQSAAVAEPLPSPRAQPPGGTPADAGPGQGSSEEEPELVFAVNLPPAQLSSSDEETREELARIGLVPPPEEFANGVLLATPLAGPGPSPTTVPSPASGKPSSEPPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASAGLASAAGPARPRYLFQRRSKLWGDPVESRGLPGPEDDKPTVISELSSRLQQLNKDTRSLGEEPVGGLGSLLDPAKKSPIAAARLFSSLGELSSISAQRSPGGPGGGASYSVRPSGRYPVARRAPSPVKPASLERVEGLGAGAGGAGRPFGLTPPTILKSSSLSIPHEPKEVRFVVRSVSARSRSPSPSPLPSPASGPGPGAPGPRRPFQQKPLQLWSKFDVGDWLESIHLGEHRDRFEDHEIEGAHLPALTKDDFVELGVTRVGHRMNIERALRQLDGS

SEQ ID NO: 2 - protein, 1740 aa, Rattus norvegicus
MDGPGASAVVVRVGIPDLQQTKCLRLDPTAPVWAAKQRVLCALNHSLQDALNYGLFQPPSRGRAGKFLDEERLLQDYPPNLDTPLPYLEFRYKRRVYAQNLIDDKQFAKLHTKANLKKFMDYVQLHSTDKVARLLDKGLDPNFHDPDSGECPLSLAAQLDNATDLLKVLRNGGAHLDFRTRDGLTAVHCATRQRNAGALTTLLDLGASPDYKDSRGLTPLYHSALGGGDALCCELLLHDHAQLGTTDENGWQEIHQACRFGHVQHLEHLLFYGANMGAQNASGNTALHICALYNQESCARVLLFRGANKDVRNYNSQTAFQVAIIAGNFELAEVIKTHKDSDVVPFRETPSYAKRRRLAGPSGLASPRPLQRSASDINLKGDQPAASPGPTLRSLPHQLLLQRLQEEKDRDRDGEQENDISGPSAGRGGHSKISPSGPGGSGPAPGPGPASPAPPAPPPRGPKRKLYSAVPGRKFIAVKAHSPQGEGEIPLHRGEAVKVLSIGEGGFWEGTVKGRTGWFPADCVEEVQMRQYDTRHETREDRTKRLFRHYTVGSYDSLTSHSDYVIDDKVAILQKRDHEGFGFVLRGAKAETPIEEFTPTPAFPALQYLESVDVEGVAWKAGLRTGDFLIEVNGVNVVKVGHKQVVGLIRQGGNRLVMKVVSVTRKPEEDSARRRAPPPPKRAPSTTLTLRSKSMTAELEELASIRRRKGEKLDEILAVAAEPTLRPDIADADSRAATVKQRPTSRRITPAEISSLFERQGLPGPEKLPGSLRKGIPRTKSVGEDEKLASLLEGRFPRSTSMQDTVREGRGIPPPPQTAPPPPPAPYYFDSGPPPTFSPPPPPPGRAYDTVRSSFKPGLEARLGAGAAGLYDSGTPLGPLPYPERQKRARSMIILQDSAPEVGDVPRPAPAATPPERPKRRPRPSGPDSPYANLGAFSASLFAPSKPQRRKSPLVKQLQVEDAQERAALAVGSPGPVGGSFAREPSPTHRGPRPGGLDYSSGEGLGLTFGGPSPGPVKERRLEERRRSTVFLSVGAIEGSPPSADLPSLQPSRSIDERLLGTGATTGRDLLLPSPVSALKPLVGGPSLGPSGSTFIHPLTGKPLDPSSPLALALAARERALASQTPSRSPTPVHSPDADRPGPLFVDVQTRDSERGPLASPAFSPRSPAWIPVPARREAEKPTREERKSPEDKKSMILSVLDTSLQRPAGLIVVHATSNGQEPNRLGAEEERPGTPELAPTPMQAAAVAEPMPSPRAQPPGSIPADPGPGQGSSEEEPELVFAVNLPPAQLSSSDEETREELARIGLVPPPEEFANGILLATPPPGPGPLPTTVPSPASGKPSSELPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHATSTGLTSAAGPARPRYLFQRRSKLWGDPVESRGLPGPEDDKPTVISELSSRLQQLNKDTRSLGEEPVGGLGSLLDPAKKSPIAAARCAVVPSAGWLFSSLGELSTISAQRSPGGPGGGASYSVRPSGRYPVARRAPSPVKPASLERVEGLGAGVGGAGRPFGLTPPTILKSSSLSIPHEPKEVRFVVRSVSARSRSPSPSPLPSPSPGSGPSAGPRRPFQQKPLQLWSKFDVGDWLESIHLGEHRDRFEDHEIEGAHLPALTKEDFVELGVTRVGHRMNIERALRQLDGS

SEQ ID NO: 3 - protein, 1730 aa, Mus musculus
MDGPGASAVVVRVGIPDLQQTKCLRLDPTAPVWAAKQRVLCALNHSLQDALNYGLFQPPSRGRAGKFLDEERLLQDYPPNLDTPLPYLEFRYKRRVYAQNLIDDKQFAKLHTKANLKKFMDYVQLHSTDKVARLLDKGLDPNFHDPDSGECPLSLAAQLDNATDLLKVLRNGGAHLDFRTRDGLTAVHCATRQRNAGALTTLLDLGASPDYKDSRGLTPLYHSALGGGDALCCELLLHDHAQLGTTDENGWQEIHQACRFGHVQHLEHLLFYGANMGAQNASGNTALHICALYNQESCARVLLFRGANKDVRNYNSQTAFQVAIIAGNFELAEVIKTHKDSDVVPFRETPSYAKRRRLAGPSGLASPRPLQRSASDINLKGDQPAASPGPTLRSLPHQLLLQRLQEEKDRDRDGELENDISGPSAGRGGHNKISPSGPGGSGPAPGPGPASPAPPAPPPRGPKRKLYSAVPGRKFIAVKAHSPQGEGEIPLHRGEAVKVLSIGEGGFWEGTVKGRTGWFPADCVEEVQMRQYDTRHETREDRTKRLFRHYTVGSYDSLTSHSDYVIDDKVAILQKRDHEGFGFVLRGAKAETPIEEFTPTPAFPALQYLESVDVEGVAWRAGLRTGDFLIEVNGVNVVKVGHKQVVGLIRQGGNRLVMKVVSVTRKPEEDGARRRAPPPPKRAPSTTLTLRSKSMTAELEELASIRRRKGEKLDEILAVAAEPTLRPDIADADSRAATVKQRPTSRRITPAEISSLFERQGLPGPEKLPGSLRKGIPRTKSVGEDEKLASLLEGRFPRSTSMQDTVREGRGIPPPPQTAPPPPPAPYYFDSGPPPTFSPPPPPGRAYDTVRSSFKPGLEARLGAGAAGLYDPSTPLGPLPYPERQKRARSMIILQDSAPEVGDVPRPAPAATPPERPKRRPRPSGPDSPYANLGAFSASLFAPSKPQRRKSPLVKQLQVEDAQERAALAVGSPGPVGGSFAREPSPTHRGPRPGSLDYSSGEGLGLTFGGPSPGPVKERRLEERRRSTVFLSVGAIEGSPPSADLPSLQPSRSIDERLLGTGATTGRDLLLPSPVSALKPLVGGPSLGPSGSTFIHPLTGKPLDPSSPLALALAARERALASQTPSRSPTPVHSPDADRPGPLFVDVQTRDSERGPLASPAFSPRSPAWIPVPARREAEKPPREERKSPEDKKSMILSVLDTSLQRPAGLIVVHATSNGQEPSRLGAEEERPGTPELAPAPMQAAAVAEPMPSPRAQPPGSIPADPGPGQGSSEEEPELVFAVNLPPAQLSSSDEETREELARIGLVPPPEEFANGILLTTPPPGPGPLPTTVPSPASGKPSSELPPAPESAADSGVEEADTRSSSDPHLETTSTISTVSSMSTLSSESGELTDTHTSFADGHTFLLEKPPVPPKPKLKSPLGKGPVTFRDPLLKQSSDSELMAQQHHAASTGLASAAGPARPRYLFQRRSKLWGDPVESRGLPGPEDDKPTVISELSSRLQQLNKDTRSLGEEPVGGLGSLLDPAKKSPIAAARLFSSLGELSTISAQRSPGGPGGGASYSVRPSGRYPVARRAPSPVKPASLERVEGLGAGVGGAGRPFGLTPPTILKSSSLSIPHEPKEVRFVVRSVSARSRSPSPSPLPSPSPGSGPSAGPRRPFQQKPLQLWSKFDVGDWLESIHLGEHRDRFEDHEIEGAHLPALTKEDFVELGVTRVGHRMNIERALRQLDGS

SEQ ID NO: 4 - RNA, 21 nt, Artificial Sequence
  siRNA
uuguaguugcggacaucccug

SEQ ID NO: 5 - RNA, 19 nt, Artificial Sequence
  siRNA
uuguagucaggugaagccc

SEQ ID NO: 6 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 7 - protein, 170 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGSRSGSSSSSGTLWDPPGPM

SEQ ID NO: 8 - protein, 189 aa, Homo sapiens [sequence omitted]

SEQ ID NO: 9 - protein, 188 aa, Homo sapiens [sequence omitted]

SEQ ID NO: 10 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 11 - protein, 93 aa, Homo sapiens
MDGPGASAVVVRVGIPDLQQTKCLRLDPAAPVWAAKQRVLCALNHSLQDALNYGLFQPPSRGRAGKFLDEERLLQEYPPNLDTPLPYLEFRYK

SEQ ID NO: 12 - protein, 93 aa, Rattus norvegicus
MDGPGASAVVVRVGIPDLQQTKCLRLDPTAPVWAAKQRVLCALNHSLQDALNYGLFQPPSRGRAGKFLDEERLLQDYPPNLDTPLPYLEFRYK

SEQ ID NO: 13 - protein, 93 aa, Mus musculus; identical to SEQ ID NO: 12

SEQ ID NO: 14 - DNA, 7345 bp, Homo sapiens
aggcgccgagctgagccggggccgatgcagctgagccgcgccgccgccgccgccgccgccgcccctgcggagcccccggagccgctgtcccccgcgccggccccggccccggccccccccggccccctcccgcgcagcgcggccgacggggctccggcgggggggaagggggggccggggcgccgcgcggagtccccgggcgctccgttccccggcgcgagcggccccggcccgggccccggcgcggggatggacggccccggggccagcgccgtggtcgtgcgcgtcggcatcccggacctgcagcagacgaagtgcctgcgcctggacccggccgcgcccgtgtgggccgccaagcagcgcgtgctctgcgccctcaaccacagcctccaggacgcgctcaactatgggcttttccagccgccctcccggggccgcgccggcaagttcctggatgaggagcggctcctgcaggagtacccgcccaacctggacacgcccctgccctacctggagtttcgatacaagcggcgagtttatgcccagaacctcatcgatgataagcagtttgcaaagcttcacacaaaggcgaacctgaagaagttcatggactacgtccagctgcatagcacggacaaggtggcacgcctgttggacaaggggctggaccccaacttccatgaccctgactcaggagagtgccccctgagcctcgcagcccagctggacaacgccacggacctgctaaaggtgctgaagaatggtggtgcccacctggacttccgcactcgcgatgggctcactgccgtgcactgtgccacacgccagcggaatgcggcagcactgacgaccctgctggacctgggggcttcacctgactacaaggacagccgcggcttgacacccctctaccacagcgccctggggggtggggatgccctctgctgtgagctgcttctccacgaccacgctcagctggggatcaccgacgagaatggctggcaggagatccaccaggcctgccgctttgggcacgtgcagcatctggagcacctgctgttctatggggcagacatgggggcccagaacgcctcggggaacacagccctgcacatctgtgccctctacaaccaggagagctgtgctcgtgtcctgctcttccgtggagctaacagggatgtccgcaactacaacagccagacagccttccaggtggccatcatcgcagggaactttgagcttgcagaggttatcaagacccacaaagactcggatgttgtaccattcagggaaacccccagctatgcgaagcggcggcgactggctggccccagtggcttggcatcccctcggcctctgcagcgctcagccagcgatatcaacctgaagggggaggcacagccagcagcttctcctggaccctcgctgagaagcctcccccaccagctgctgctccagcggctgcaagaggagaaagatcgtgaccgggatgccgaccaggagagcaacatcagtggccctttagcaggcagggccggccaaagcaagatcagcccgagcgggcccggcggccccggccccgcgcccggccccggccccgcgccccctgcgccccccgcaccgccgccccggggcccgaagcggaaactttacagcgccgtccccggccgcaagttcatcgccgtgaaggcgcacagcccgcagggtgaaggcgagatcccgctgcaccgcggcgaggccgtgaaggtgctcagcattggggagggcggtttctgggagggaaccgtgaaaggccgcacgggctggttcccggccgactgcgtggaggaagtgcagatgaggcagcatgacacacggcctgaaacgcgggaggaccggacgaagcggctctttcggcactacacagtgggctcctacgacagcctcacctcacacagcgattatgtcattgatgacaaagtggctgtcctgcagaaacgggaccacgagggctttggttttgtgctccggggagccaaagcagagacccccatcgaggagttcacgcccacgccagccttcccggcgctgcagtatctcgagtcggtggacgtggagggtgtggcctggagggccgggctgcgcacgggagacttcctcatcgaggtgaacggggtgaacgtggtgaaggtcggacacaagcaggtggtggctctgattcgccagggtggcaaccgcctcgtcatgaaggttgtgtctgtgacaaggaagccagaagaggacggggctcggcgcagagccccaccgccccccaagagggcccccagcaccacactgaccctgcgctccaagtccatgacagctgagctcgaggaacttgcctccattcggagaagaaaaggggagaagctggacgagatgctggcagccgccgcagagccaacgctgcggccagacatcgcagacgcagactccagagccgccaccgtcaaacagaggcccaccagtcggaggatcacacccgccgagattagctcattgtttgaacgccagggcctcccaggcccagagaagctgccgggctccttgcggaaggggattccacggaccaagtctgtaggggaggacgagaagctggcgtccctgctggaagggcgcttcccgcggagcacctcgatgcaagacccggtgcgcgagggtcgcggcatcccgcccccgccgcagaccgcgccgcctcccccgcccgcgccctactacttcgactcggggccgcccccggccttctcgccgccgcccccgccgggccgcgcctacgacacggtgcgctccagcttcaagcccggcctggaggcgcgcctgggcgcgggcgctgccggcctgtacgagccgggcgcggccctcggcccgctgccgtatcccgagcggcagaagcgcgcgcgctccatgatcatcctgcaggactcggcgcccgagtcgggcgacgcccctcgacccccgcccgcggccaccccgcccgagcgacccaagcgccggccgcggccgcccggccccgacagcccctacgccaacctgggcgccttcagcgccagcctcttcgctccgtccaagccgcagcgccgcaagagccccctggtgaagcagctgcaggtggaggacgcgcaggagcgcgcggccctggccgtgggcagccccggtcccggcggcggcagcttcgcccgcgagccctccccgacccaccgcggtccgcgcccgggtggcctcgactacggcgcgggcgatggcccggggctcgcgttcggcggcccgggcccggccaaggaccggcggctggaggagcggcgccgctccactgtgttcctgtccgtgggggccatcgagggcagcgcccccggcgcggatctgccatccctacagccctcccgctccatcgacgagcgcctcctggggaccggccccaccgccggccgcgacctgctgctgccctccccggtgtctgccctgaagccgttggtcagcggcccgagcctggggccctcgggttccaccttcatccacccactcaccggcaaacccctggaccccagctcacccctggcccttgccctggctgcccgagagcgagctctggcctcccaggcgccctcccggtcccccacacccgtgcacagtcccgacgccgaccgccccggacccctgtttgtggatgtacaggcccgggacccagagcgagggtccctggcttccccggctttctccccacggagcccagcctggattcctgtgcctgctcgcagggaggcagagaaggtcccccgggaggagcggaagtcacccgaggacaagaagtccatgatcctcagcgtcctggacacatccctgcagcggccagctggcctcatcgttgtgcacgccaccagcaacgggcaggagcccagcaggctggggggggccgaagaggagcgcccgggcaccccggagttggccccggcccccatgcagtcagcggctgtggcagagcccctgcccagcccccgggcccagccccctggtggcaccccggcagacgccgggccaggccagggcagctcagaggaagagccagagctggtgtttgctgtgaacctgccacctgcccagctgtcgtccagcgatgaggagaccagggaggagctggcccgaattgggttggtgccaccccctgaagagtttgccaacggggtcctgctggccaccccactcgctggcccgggcccctcgcccaccacggtgcccagcccggcctcagggaagcccagcagtgagccaccccctgcccctgagtctgcagccgactctggggtggaggaggctgacacacgcagctccagcgacccccacctggagaccacaagcaccatctccacggtgtccagcatgtccaccttgagctcggagagcggggaactcactgacacccacacctccttcgctgacggacacacttttctactcgagaagccaccagtgcctcccaagcccaagctcaagtccccgctggggaaggggccggtgaccttcagggacccgctgctgaagcagtcctcggacagcgagctcatggcccagcagcaccacgccgcctctgccgggctggcctctgccgccgggcctgcccgccctcgctacctcttccagagaaggtccaagctatggggggaccccgtggagagccgggggctccctgggcctgaagacgacaaaccaactgtgatcagtgagctcagctcccgcctgcagcagctgaacaaggacacgcgttccctgggggaggaaccagttggtggcctgggcagcctgctggaccctgccaagaagtcgcccatcgcagcagctcggctcttcagcagcctcggtgagctgagctccatttcagcgcagcgcagccccgggggcccgggcggcggggcctcgtactcggtgaggcccagtggccgctaccccgtggcgagacgcgccccgagcccggtgaagcccgcgtcgctggagcgggtggaggggctgggggcgggcgcggggggcgcagggcggcccttcggcctcacgccccccaccatcctcaagtcgtccagcctctccatcccgcacgagcccaaggaggtgcgcttcgtggtgcgcagcgtgagcgcgcgcagtcgctccccctcgccgtcgccgctgccctcgcccgcgtccggccccggccccggcgcccccggcccacgccgacccttccagcagaagccgctgcagctctggagcaagttcgacgtgggcgactggctggagagcatccacctaggcgagcaccgcgaccgcttcgaggaccatgagatagaaggcgcgcacctacccgcgcttaccaaggacgacttcgtggagctgggcgtcacgcgcgtgggccaccgcatgaacatcgagcgcgcgctcaggcagctggacggcagctgacgccccacccccactcccgccccggccgtgccctgccggcagggccccccacccccaccccgggccgcgggctcggcctgccccttacgacggcgcccgggccaggaatgttgcatgaatcgtcctgtttgctgttgctcggagactcgccctgtacattgcttagtgccctcaccggccgcccagcccacccagcgcacagtcaggaagggcgtggaccagggaggctggggcgggaggtgccgggggtggggtgccctagcgtgaccacctccttcgcagctcctggtggccattctcccagagggggaacctagtccagcatgcgaggtcaggacccgccttggtgactcggggggaggggggagacattgggattctcgatgggggccaaggagcccccctgttttgcatattttaatccactctatatttggaacgagaaaaggaacaaatatctctgtccgtaatagtttcctctcccctcccttctacttccactggtcccactgcagctgcccagtcttccatctccggcccctcactgccactgccaccccacaacggggcaggggacgctccagctggtctggggttggccagggccctagtggcccgccctggggccccagctcggcccctcgcctcgctgagctctagtgtgccccaccgacccttcaggtgctgctcgtggtgggaggggcggcaggccgcgggtcctgctgtgcacccgcgggaccagccggcctgggagaccatcggccgggggggatgagggcagggccctgccgctccaccgcagccatcttcctcacagggtctctccccaaggagggggctagcttggtccccatgctcttgggcaactacagcagagaagcctccctgccttggaccccaaagtctcctgtcctgccctttatgtgtgtgggtgaaactgggtgcgtctgagcacgtgggagccgtgtgtgtgcctgattactgagtggccaccaggggccgctctggactagcgcggggccgtggaggcgtgcaccgtgtgcatgcgtggggtgtacctgtgagagcaccctgtctcctcttccaaagaaagtcagaggccatcctgcaccctgggtccagctgtttgcccagcctgtccttccagagcctcacccagcctgagcggggttccctggtgaatccctgctgcttggggaggccccaagggccccttggaggcagcgcccccaccttgggcttctgagggcatcatagggggacccctagagtcagttcaccacaggccctggggagagtcaaagacccccgagggtgcccagccccccacactgtgactcctcacactcagcgatgacctgtggggtggggggccctgggacgtttttaaacctagggtttggagtctggactaagctccatccacgtcactcacaagtttctgtttatatttctagctttttttaataaaataaaaaaaaaaagaaaacagaagttttcacaacccaggggcctggcacgccggtctgtgcctgcccgccccgccctggcccaccggccccactccctgggcacagagtcacacccactcatccttccgccaacagtccaggtcacacagcagcagtcactgtaacagactgccacatacacactcggtctcacactcacctgtgggttttggttccgttcaatttgggtttttaactttacagggtcagttccgcttcacctccttttgtatggagttccatccggggggtttcaccccctgctccagtcctgaggcctcctgaccctgacgttgtgatacgccccacagagatctatgtttcttatattattattattgataataattattataatattattatgtaataaatttataagaaatgaa

SEQ ID NO: 15 - protein, 189 aa, Homo sapiens
  MISC_FEATURE (5)..(5): Xaa can be Lys or Glu
  MISC_FEATURE (12)..(12): Xaa can be Gly, Ala, Cys, Asp, Arg, Ser or Val
  MISC_FEATURE (13)..(13): Xaa can be Gly, Cys, Asp or Val
  MISC_FEATURE (14)..(14): Xaa can be Val or Ile
  MISC_FEATURE (19)..(19): Xaa can be Leu or Phe
  MISC_FEATURE (22)..(22): Xaa can be Gln or Lys
  MISC_FEATURE (33)..(33): Xaa can be Asp or Glu
  MISC_FEATURE (36)..(36): Xaa can be Ile or Met
  MISC_FEATURE (58)..(58): Xaa can be Thr or Lys
  MISC_FEATURE (59)..(59): Xaa can be Ala, Gly, Glu or Thr
  MISC_FEATURE (61)..(61): Xaa can be Gln, His, Leu, Arg, Glu or Lys
  MISC_FEATURE (62)..(62): Xaa can be Glu or Lys
  MISC_FEATURE (63)..(63): Xaa can be Glu or Lys
  MISC_FEATURE (68)..(68): Xaa can be Arg, Met or Ser
  MISC_FEATURE (71)..(71): Xaa can be Tyr or Cys
  MISC_FEATURE (92)..(92): Xaa can be Asp or Tyr
  MISC_FEATURE (117)..(117): Xaa can be Lys or Asn
  MISC_FEATURE (146)..(146): Xaa can be Ala, Thr, Val or Pro
MTEYXLVVVGAXXXGKSAXTIXLIQNHFVDEYXPTXEDSYRKQVVIDGETCLLDILDXXGXXXYSAMXDQXMRTGEGFLCVFAINNTKSFEXIHHYREQIKRVKDSEDVPMVLVGNXCDLPSRTVDTKQAQDLARSYGIPFIETSXKTRQRVEDAFYTLVREIRQYRLKKISKEEKTPGCVKIKKCIIM

SEQ ID NO: 16 - protein, 188 aa, Homo sapiens
  MISC_FEATURE (5)..(5): Xaa can be Lys or Glu
  MISC_FEATURE (12)..(12): Xaa can be Gly, Ala, Cys, Asp, Arg, Ser or Val
  MISC_FEATURE (13)..(13): Xaa can be Gly, Cys, Asp or Val
  MISC_FEATURE (14)..(14): Xaa can be Val or Ile
  MISC_FEATURE (19)..(19): Xaa can be Leu or Phe
  MISC_FEATURE (22)..(22): Xaa can be Gln or Lys
  MISC_FEATURE (33)..(33): Xaa can be Asp or Glu
  MISC_FEATURE (36)..(36): Xaa can be Ile or Met
  MISC_FEATURE (58)..(58): Xaa can be Thr or Lys
  MISC_FEATURE (59)..(59): Xaa can be Ala, Gly, Glu or Thr
  MISC_FEATURE (61)..(61): Xaa can be Gln, His, Leu, Arg, Glu or Lys
  MISC_FEATURE (62)..(62): Xaa can be Glu or Lys
  MISC_FEATURE (63)..(63): Xaa can be Glu or Lys
  MISC_FEATURE (68)..(68): Xaa can be Arg, Met or Ser
  MISC_FEATURE (71)..(71): Xaa can be Tyr or Cys
  MISC_FEATURE (92)..(92): Xaa can be Asp or Tyr
  MISC_FEATURE (117)..(117): Xaa can be Lys or Asn
  MISC_FEATURE (146)..(146): Xaa can be Ala, Thr, Val or Pro
MTEYXLVVVGAXXXGKSAXTIXLIQNHFVDEYXPTXEDSYRKQVVIDGETCLLDILDXXGXXXYSAMXDQXMRTGEGFLCVFAINNTKSFEXIHHYREQIKRVKDSEDVPMVLVGNXCDLPSRTVDTKQAQDLARSYGIPFIETSXKTRQGVDDAFYTLVREIRKHKEKMSKDGKKKKKKSKTKCVIM

SEQ ID NO: 17 - RNA, 19 nt, Artificial Sequence
  siRNA/shRNA, target-specific anti-sense sequence in 5′->3′ direction
auaaacucgccgcuuguau

SEQ ID NO: 18 - RNA, 19 nt, Artificial Sequence
  siRNA/shRNA, target-specific anti-sense sequence in 5′ -> 3′ direction
aaaagcccauaguugagcg

SEQ ID NO: 19 - RNA, 21 nt, Artificial Sequence
  siRNA/shRNA, target-specific anti-sense sequence in 5′ -> 3′ direction
uaaacucgccgcuuguaucga

SEQ ID NO: 20 - RNA, 21 nt, Artificial Sequence
  siRNA/shRNA, target-specific anti-sense sequence in 5′ -> 3′ direction
uuaucaucgaugagguucugg

SEQ ID NO: 21 - RNA, 21 nt, Artificial Sequence
  siRNA/shRNA, target-specific anti-sense sequence in 5′ -> 3′ direction
auaaucgcugugugaggugag

SEQ ID NO: 22 - RNA, 21 nt, Artificial Sequence
  siRNA/shRNA, target-specific anti-sense sequence in 5′ -> 3′ direction
uauuacggacagagauauuug

SEQ ID NO: 23 - RNA, 21 nt, Artificial Sequence
  siRNA/shRNA, target-specific anti-sense sequence in 5′ -> 3′ direction
caaacaaugagcuaaucucgg

SEQ ID NO: 24 - RNA, 19 nt, Artificial Sequence
  siRNA/shRNA, target-specific anti-sense sequence in 5′ -> 3′ direction
ugguacaacauccgagucu

SEQ ID NO: 25 - RNA, 19 nt, Artificial Sequence
  siRNA/shRNA, target-specific anti-sense sequence in 5′ -> 3′ direction
gaacuucuucagguucgcc

SEQ ID NO: 26 - RNA, 19 nt, Artificial Sequence
  siRNA/shRNA, target-specific anti-sense sequence in 5′ -> 3′ direction
gacguaguccaugaacuuc

SEQ ID NO: 27 - RNA, 19 nt, Artificial Sequence
  siRNA/shRNA, target-specific anti-sense sequence in 5′ -> 3′ direction
uucuccgaauggaggcaag

SEQ ID NO: 28 - RNA, 25 nt, Artificial Sequence
  siRNA/shRNA, target-specific anti-sense sequence in 5′ -> 3′ direction
uguaguugcggacaucccuguuagc

SEQ ID NO: 29 - RNA, 25 nt, Artificial Sequence
  siRNA/shRNA, target-specific anti-sense sequence in 5′ -> 3′ direction
ucugcaagcucaaaguucccugcga

SEQ ID NO: 30 - RNA, 25 nt, Artificial Sequence
  siRNA/shRNA, target-specific anti-sense sequence in 5′ -> 3′ direction
aaccucugcaagcucaaaguucccu

SEQ ID NO: 31 - RNA, 25 nt, Artificial Sequence
  siRNA/shRNA, target-specific anti-sense sequence in 5′ -> 3′ direction
agggaacuuugagcuugcagagguu

SEQ ID NO: 32 - RNA, 25 nt, Artificial Sequence
  siRNA/shRNA, target-specific anti-sense sequence in 5′ -> 3′ direction
uuugucaucaaugacauaaucgcug

SEQ ID NO: 33 - RNA, 25 nt, Artificial Sequence
  siRNA/shRNA, target-specific anti-sense sequence in 5′ -> 3′ direction
aaucagagccaccaccugcuugugu

SEQ ID NO: 34 - RNA, 25 nt, Artificial Sequence
  siRNA/shRNA, target-specific anti-sense sequence in 5′ -> 3′ direction
aauggaggcaaguuccucgagcuca

SEQ ID NO: 35 - RNA, 25 nt, Artificial Sequence
  siRNA/shRNA, target-specific anti-sense sequence in 5′ -> 3′ direction
caaacaaugagcuaaucucggcggg

SEQ ID NO: 36 - protein, 1806 aa, Homo sapiens [sequence omitted]

SEQ ID NO: 37 - DNA, 7691 bp, Homo sapiens
acacaccgcactggaggctccgctctgcccgctccgggaccccttcctccgccgcacccgccccggtggtccgcggatgcccgcccttgccgctcagccactccccccgcaccgaggcctaggactcccccccccaaccccgtcacagccccccagacccccgccccgtggctcggcccccgccctccgcacacacctcccgcccccacccgggaccccgcaagtaaccccccagcactggccctgagccctcccggcccccgcctccggcgcagccccctcgccacccccgcttccctcccgtctcaggccccctccccccgccgcccccgcccccggggaaggcaggcgccgagctgagccggggccgatgcagctgagccgcgccgccgccgccgccgccgccgcccctgcggagcccccggagccgctgtcccccgcgccggccccggccccggccccccccggccccctcccgcgcagcgcggccgacggggctccggcgggggggaagggggggccggggcgccgcgcggagtccccgggcgctccgttccccggcgcgagcggccccggcccgggccccggcgcggggatggacggccccggggccagcgccgtggtcgtgcgcgtcggcatcccggacctgcagcagacgaagtgcctgcgcctggacccggccgcgcccgtgtgggccgccaagcagcgcgtgctctgcgccctcaaccacagcctccaggacgcgctcaactatgggcttttccagccgccctcccggggccgcgccggcaagttcctggatgaggagcggctcctgcaggagtacccgcccaacctggacacgcccctgccctacctggagtttcgatacaagcggcgagtttatgcccagaacctcatcgatgataagcagtttgcaaagcttcacacaaaggcgaacctgaagaagttcatggactacgtccagctgcatagcacggacaaggtggcacgcctgttggacaaggggctggaccccaacttccatgaccctgactcaggagagtgccccctgagcctcgcagcccagctggacaacgccacggacctgctaaaggtgctgaagaatggtggtgcccacctggacttccgcactcgcgatgggctcactgccgtgcactgtgccacacgccagcggaatgcggcagcactgacgaccctgctggacctgggggcttcacctgactacaaggacagccgcggcttgacacccctctaccacagcgccctggggggtggggatgccctctgctgtgagctgcttctccacgaccacgctcagctggggatcaccgacgagaatggctggcaggagatccaccaggcctgccgctttgggcacgtgcagcatctggagcacctgctgttctatggggcagacatgggggcccagaacgcctcggggaacacagccctgcacatctgtgccctctacaaccaggagagctgtgctcgtgtcctgctcttccgtggagctaacagggatgtccgcaactacaacagccagacagccttccaggtggccatcatcgcagggaactttgagcttgcagaggttatcaagacccacaaagactcggatgttgtaccattcagggaaacccccagctatgcgaagcggcggcgactggctggccccagtggcttggcatcccctcggcctctgcagcgctcagccagcgatatcaacctgaagggggaggcacagccagcagcttctcctggaccctcgctgagaagcctcccccaccagctgctgctccagcggctgcaagaggagaaagatcgtgaccgggatgccgaccaggagagcaacatcagtggccctttagcaggcagggccggccaaagcaagatcagcccgagcgggcccggcggccccggccccgcgcccggccccggccccgcgccccctgcgccccccgcaccgccgccccggggcccgaagcggaaactttacagcgccgtccccggccgcaagttcatcgccgtgaaggcgcacagcccgcagggtgaaggcgagatcccgctgcaccgcggcgaggccgtgaaggtgctcagcattggggagggcggtttctgggagggaaccgtgaaaggccgcacgggctggttcccggccgactgcgtggaggaagtgcagatgaggcagcatgacacacggcctgaaacgcgggaggaccggacgaagcggctctttcggcactacacagtgggctcctacgacagcctcacctcacacagcgattatgtcattgatgacaaagtggctgtcctgcagaaacgggaccacgagggctttggttttgtgctccggggagccaaagcagagacccccatcgaggagttcacgcccacgccagccttcccggcgctgcagtatctcgagtcggtggacgtggagggtgtggcctggagggccgggctgcgcacgggagacttcctcatcgaggtgaacggggtgaacgtggtgaaggtcggacacaagcaggtggtggctctgattcgccagggtggcaaccgcctcgtcatgaaggttgtgtctgtgacaaggaagccagaagaggacggggctcggcgcagagccccaccgccccccaagagggcccccagcaccacactgaccctgcgctccaagtccatgacagctgagctcgaggaacttgcctccattcggagaagaaaaggggagaagctggacgagatgctggcagccgccgcagagccaacgctgcggccagacatcgcagacgcagactccagagccgccaccgtcaaacagaggcccaccagtcggaggatcacacccgccgagattagctcattgtttgaacgccagggcctcccaggcccagagaagctgccgggctccttgcggaaggggattccacggaccaagtctgtaggggaggacgagaagctggcgtccctgctggaagggcgcttcccgcggagcacctcgatgcaagacccggtgcgcgagggtcgcggcatcccgcccccgccgcagaccgcgccgcctcccccgcccgcgccctactacttcgactcggggccgcccccggccttctcgccgccgcccccgccgggccgcgcctacgacacggtgcgctccagcttcaagcccggcctggaggcgcgcctgggcgcgggcgctgccggcctgtacgagccgggcgcggccctcggcccgctgccgtatcccgagcggcagaagcgcgcgcgctccatgatcatcctgcaggactcggcgcccgagtcgggcgacgcccctcgacccccgcccgcggccaccccgcccgagcgacccaagcgccggccgcggccgcccggccccgacagcccctacgccaacctgggcgccttcagcgccagcctcttcgctccgtccaagccgcagcgccgcaagagccccctggtgaagcagctgcaggtggaggacgcgcaggagcgcgcggccctggccgtgggcagccccggtcccggcggcggcagcttcgcccgcgagccctccccgacccaccgcggtccgcgcccgggtggcctcgactacggcgcgggcgatggcccggggctcgcgttcggcggcccgggcccggccaaggaccggcggctggaggagcggcgccgctccactgtgttcctgtccgtgggggccatcgagggcagcgcccccggcgcggatctgccatccctacagccctcccgctccatcgacgagcgcctcctggggaccggccccaccgccggccgcgacctgctgctgccctccccggtgtctgccctgaagccgttggtcagcggcccgagcctggggccctcgggttccaccttcatccacccactcaccggcaaacccctggaccccagctcacccctggcccttgccctggctgcccgagagcgagctctggcctcccaggcgccctcccggtcccccacacccgtgcacagtcccgacgccgaccgccccggacccctgtttgtggatgtacaggcccgggacccagagcgagggtccctggcttccccggctttctccccacggagcccagcctggattcctgtgcctgctcgcagggaggcagagaaggtcccccgggaggagcggaagtcacccgaggacaagaagtccatgatcctcagcgtcctggacacatccctgcagcggccagctggcctcatcgttgtgcacgccaccagcaacgggcaggagcccagcaggctggggggggccgaagaggagcgcccgggcaccccggagttggccccggcccccatgcagtcagcggctgtggcagagcccctgcccagcccccgggcccagccccctggtggcaccccggcagacgccgggccaggccagggcagctcagaggaagagccagagctggtgtttgctgtgaacctgccacctgcccagctgtcgtccagcgatgaggagaccagggaggagctggcccgaattgggttggtgccaccccctgaagagtttgccaacggggtcctgctggccaccccactcgctggcccgggcccctcgcccaccacggtgcccagcccggcctcagggaagcccagcagtgagccaccccctgcccctgagtctgcagccgactctggggtggaggaggctgacacacgcagctccagcgacccccacctggagaccacaagcaccatctccacggtgtccagcatgtccaccttgagctcggagagcggggaactcactgacacccacacctccttcgctgacggacacacttttctactcgagaagccaccagtgcctcccaagcccaagctcaagtccccgctggggaaggggccggtgaccttcagggacccgctgctgaagcagtcctcggacagcgagctcatggcccagcagcaccacgccgcctctgccgggctggcctctgccgccgggcctgcccgccctcgctacctcttccagagaaggtccaagctatggggggaccccgtggagagccgggggctccctgggcctgaagacgacaaaccaactgtgatcagtgagctcagctcccgcctgcagcagctgaacaaggacacgcgttccctgggggaggaaccagttggtggcctgggcagcctgctggaccctgccaagaagtcgcccatcgcagcagctcggctcttcagcagcctcggtgagctgagctccatttcagcgcagcgcagccccgggggcccgggcggcggggcctcgtactcggtgaggcccagtggccgctaccccgtggcgagacgcgccccgagcccggtgaagcccgcgtcgctggagcgggtggaggggctgggggcgggcgcggggggcgcagggcggcccttcggcctcacgccccccaccatcctcaagtcgtccagcctctccatcccgcacgagcccaaggaggtgcgcttcgtggtgcgcagcgtgagcgcgcgcagtcgctccccctcgccgtcgccgctgccctcgcccgcgtccggccccggccccggcgcccccggcccacgccgacccttccagcagaagccgctgcagctctggagcaagttcgacgtgggcgactggctggagagcatccacctaggcgagcaccgcgaccgcttcgaggaccatgagatagaaggcgcgcacctacccgcgcttaccaaggacgacttcgtggagctgggcgtcacgcgcgtgggccaccgcatgaacatcgagcgcgcgctcaggcagctggacggcagctgacgccccacccccactcccgccccggccgtgccctgccggcagggccccccacccccaccccgggccgcgggctcggcctgccccttacgacggcgcccgggccaggaatgttgcatgaatcgtcctgtttgctgttgctcggagactcgccctgtacattgcttagtgccctcaccggccgcccagcccacccagcgcacagtcaggaagggcgtggaccagggaggctggggcgggaggtgccgggggtggggtgccctagcgtgaccacctccttcgcagctcctggtggccattctcccagagggggaacctagtccagcatgcgaggtcaggacccgccttggtgactcggggggaggggggagacattgggattctcgatgggggccaaggagcccccctgttttgcatattttaatccactctatatttggaacgagaaaaggaacaaatatctctgtccgtaatagtttcctctcccctcccttctacttccactggtcccactgcagctgcccagtcttccatctccggcccctcactgccactgccaccccacaacggggcaggggacgctccagctggtctggggttggccagggccctagtggcccgccctggggccccagctcggcccctcgcctcgctgagctctagtgtgccccaccgacccttcaggtgctgctcgtggtgggaggggcggcaggccgcgggtcctgctgtgcacccgcgggaccagccggcctgggagaccatcggccgggggggatgagggcagggccctgccgctccaccgcagccatcttcctcacagggtctctccccaaggagggggctagcttggtccccatgctcttgggcaactacagcagagaagcctccctgccttggaccccaaagtctcctgtcctgccctttatgtgtgtgggtgaaactgggtgcgtctgagcacgtgggagccgtgtgtgtgcctgattactgagtggccaccaggggccgctctggactagcgcggggccgtggaggcgtgcaccgtgtgcatgcgtggggtgtacctgtgagagcaccctgtctcctcttccaaagaaagtcagaggccatcctgcaccctgggtccagctgtttgcccagcctgtccttccagagcctcacccagcctgagcggggttccctggtgaatccctgctgcttggggaggccccaagggccccttggaggcagcgcccccaccttgggcttctgagggcatcatagggggacccctagagtcagttcaccacaggccctggggagagtcaaagacccccgagggtgcccagccccccacactgtgactcctcacactcagcgatgacctgtggggtggggggccctgggacgtttttaaacctagggtttggagtctggactaagctccatccacgtcactcacaagtttctgtttatatttctagctttttttaataaaataaaaaaaaaaagaaaacagaagttttcacaacccaggggcctggcacgccggtctgtgcctgcccgccccgccctggcccaccggccccactccctgggcacagagtcacacccactcatccttccgccaacagtccaggtcacacagcagcagtcactgtaacagactgccacatacacactcggtctcacactcacctgtgggttttggttccgttcaatttgggtttttaactttacagggtcagttccgcttcacctccttttgtatggagttccatccggggggtttcaccccctgctccagtcctgaggcctcctgaccctgacgttgtgatacgccccacagagatctatgtttcttatattattattattgataataattattataatattattatgtaataaatttataagaaatgaa